(19) United States
(12) Patent Application Publication
(10) Pub. No.: US 2025/0049617 A1
(43) Pub. Date: Feb. 13, 2025

(54) ABSORBENT ARTICLES WITH LOW VOID VOLUME

(71) Applicant: Kimberly-Clark Worldwide, Inc., Neenah, WI (US)

(72) Inventors: Kate T Bowen, Littleton, MA (US); WanDuk Lee, Appleton, WI (US); Mark Beitz, Appleton, WI (US); Neil Scholl, Neenah, WI (US)

(21) Appl. No.: 18/719,049

(22) PCT Filed: Dec. 15, 2021
(86) PCT No.: PCT/US21/63498

§ 371 Date: Jun. 12, 2024

(51) Int. Cl. A61F 13/531

(52) U.S. Cl. CPC A61F 13/531

(57) ABSTRACT

Absorbent articles with low void volumes and fast fluid intake properties are described. In one embodiment, an absorbent article has a liner, backsheet, and absorbent structure disposed therebetween. The absorbent structure may comprise top and bottom corewrap materials with superabsorbent disposed therebetween with superabsorbent particles disposed at a basis weight of greater than or equal to 250 gsm and present in an amount greater than 90% by weight of absorbent material within the absorbent article. The superabsorbent particles may have a vortex time of less than 41 s, according to the Vortex Time Test Method, the absorbent article may have a dry void volume of less than 0.45 cm³/cm², as determined by the Dry Void Volume Determination Test Method, and the absorbent article may have a first cradle intake time of less than 20 s, according to the Cradle Intake Test Method

### TECHNICAL FIELD

The present disclosure is directed to absorbent bodies and more specifically to absorbent bodies with a high superabsorbent material content and low dry void volumes.

### BACKGROUND OF THE DISCLOSURE

A primary function of personal care absorbent articles is to absorb and retain body exudates such as urine, fecal material, blood, and menses with additional desired attributes including low leakage of the exudates from the absorbent article and a dry feel to the wearer of the absorbent article. By preventing leakage of the exudates from the absorbent article, the absorbent article intends to prevent the body exudates from soiling or contaminating a wearer's or caregiver's clothing or other articles, such as bedding, that can come in contact with the wearer.

Absorbent bodies typically help with fluid uptake and storage within absorbent articles. Many absorbent bodies contain multiple absorbent materials such as superabsorbent material and pulp fluff or other fibrous absorbent material. Each type of absorbent material helps to impart such absorbent bodies with a range of properties useful in absorbing and retaining liquid bodily exudates. For example, pulp fluff or other fibrous absorbent material may absorb liquid more quickly than superabsorbent material, and the superabsorbent material may be able retain more liquid per particle than pulp fluff.

Many advances have been made to absorbent bodies and absorbent articles containing such absorbent bodies—particularly to the superabsorbent material of absorbent bodies. Some current absorbent bodies may now have absorbent material comprising mostly superabsorbent material and further comprising only a small portion of other absorbent material. Other current absorbent bodies comprise only superabsorbent material as the absorbent material. Such absorbent bodies can allow absorbent articles to be made to be thinner and more flexible than articles which have absorbent bodies that contain traditionally higher amounts of traditional pulp fluff absorbent material. However, further fluid handling improvements to such low-pulp absorbent articles are a continued area of exploration to increase their performance.

### SUMMARY OF THE DISCLOSURE

In one embodiment, an absorbent article may comprise a bodyside liner, a backsheet; and an absorbent structure disposed between the bodyside liner and the backsheet and comprising a top corewrap material and a bottom corewrap material with superabsorbent particles disposed between the top corewrap material and the bottom corewrap material, the superabsorbent particles disposed at a basis weight of greater than or equal to 250 gsm and present in an amount greater than or equal to 90% by weight of absorbent material within the absorbent article, wherein the superabsorbent particles have a vortex time of less than or equal to 41 s, according to the Vortex Time Test Method, the absorbent article has a dry void volume of less than 0.45 cm³/cm², as determined by the Dry Void Volume Determination Test Method, and the absorbent article has a first cradle intake time of less than or equal to 20 s, according to the Cradle Intake Test Method.

In another embodiment, an absorbent article may comprise a bodyside liner, a backsheet, and an absorbent structure disposed between the bodyside liner and the backsheet and comprising a top corewrap material and a bottom corewrap material with superabsorbent particles and adhesive disposed between the top corewrap material and the bottom corewrap material forming one or more absorbent layers, the superabsorbent particles disposed at a basis weight of greater than or equal to 250 gsm and present in an amount greater than or equal to 90% by weight of absorbent material of the absorbent article, wherein the superabsorbent particles have a vortex time of less than or equal to 41 s, according to the Vortex Time Test Method, the absorbent article has a dry void volume of less than 0.45 cm³/cm² and an absorbent structure dry void volume of less than or equal to 0.19 cm³/cm², as determined by the Dry Void Volume Determination Test Method, and wherein the absorbent article has a first cradle intake time of less than or equal to 25 s, according to the Cradle Intake Test Method.

In a further embodiment, an absorbent article may comprise a bodyside liner, a backsheet, and an absorbent structure disposed between the bodyside liner and the backsheet and comprising a top corewrap material and a bottom corewrap material with superabsorbent particles and adhesive disposed between the top corewrap material and the bottom corewrap material forming one or more absorbent layers, the superabsorbent particles disposed at a basis weight of greater than or equal to 250 gsm and present in an amount greater than or equal to 90% by weight of absorbent material of the absorbent article, wherein the superabsorbent particles have a vortex time of less than or equal to 41 s, according to the Vortex Time Test Method, the absorbent article has a dry void volume of less than 0.45 cm³/cm², as determined by the Dry Void Volume Determination Test Method, and wherein a combined dry void volume value of the one or more absorbent layers increases by at least 314% at 40% saturation, according to the Percent Void Volume Increase Test Method.

### BRIEF DESCRIPTION OF DRAWINGS

A full and enabling disclosure thereof, directed to one of ordinary skill in the art, is set forth more particularly in the remainder of the specification, which makes reference to the appended figures in which:

FIG. 1 is side perspective view of an exemplary embodiment of an absorbent article, such as a diaper, in a fastened condition.

FIG. 2 is a top plan view of the absorbent article of FIG. 1 in a stretched, laid flat, unfastened condition.

FIG. 3 is a front perspective view of an alternative embodiment of an absorbent article, such as a pant.

FIG. 4 is a top plan view of the absorbent article of FIG. 3 in a stretched, laid flat condition.

FIG. 5 is a front perspective cross-sectional view taken along line 5-5 from FIG. 2, with the absorbent article being in a relaxed configuration.

FIG. 6 is a process schematic depicting an exemplary method of manufacturing an absorbent body according to the present disclosure.

FIG. 7 is a process schematic depicting a portion of the exemplary method of FIG. 6.

FIG. 8 is a process schematic depicting an alternative exemplary method of manufacturing an absorbent body according to the present disclosure.

FIG. 9A-9C are exemplary front cross-sectional views of an absorbent body formed according to a method of manufacturing according to aspects of the present disclosure, taken along line 9-9 from FIG. 6.

FIGS. 10A & 10B are alternative exemplary front cross-sectional views of an absorbent body formed according to a method of manufacturing according to aspects of the present disclosure, taken along line 10-10 from FIG. 8.

FIG. 11A is a top perspective view of a three-dimensional image generated by a micro-CT process used to analyze an exemplary mixture of particles and adhesive filaments formed by the process of FIG. 9, according to aspects of the present disclosure.

FIG. 11B is a top plan view of the three-dimensional image of FIG. 11A.

FIG. 11C is a cross-sectional view of a slice of the three-dimensional image of FIG. 11A.

FIG. 11D is the cross-sectional view of FIG. 11C without the particles, leaving only the adhesive filaments.

FIG. 12 is a graph depicting first fluid intake times vs. dry void volumes for exemplary absorbent articles according to the present disclosure.

Repeat use of reference characters in the present specification and drawings is intended to represent the same or analogous features or elements of the disclosure.

### DETAILED DESCRIPTION OF THE DISCLOSURE

In an embodiment, the present disclosure is generally directed towards absorbent bodies with absorbent material comprising a high proportion of superabsorbent material. The present disclosure further describes absorbent articles utilizing such absorbent bodies and beneficial properties of such absorbent articles to help promote both skin-dryness and comfort and fit properties of such absorbent articles. Each example is provided by way of explanation and is not meant as a limitation. For example, features illustrated or described as part of one embodiment or example or figure can be used on another embodiment or example or figure to yield yet another embodiment. It is intended that the present disclosure include such modifications and variations.

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements. Many modifications and variations of the present disclosure can be made without departing from the spirit and scope thereof. Therefore, the exemplary embodiments described above should not be used to limit the scope of the invention.

### Definitions

The term “absorbent article” refers herein to an article which may be placed against or in proximity to the body (i.e., contiguous with the body) of the wearer to absorb and contain various liquid, solid, and semi-solid exudates discharged from the body. Such absorbent articles, as described herein, are intended to be discarded after a limited period of use instead of being laundered or otherwise restored for reuse. It is to be understood that the present disclosure is applicable to various disposable absorbent articles, including, but not limited to, diapers, diaper pants, training pants, absorbent inserts, youth pants, swim pants, feminine hygiene products, including, but not limited to, menstrual pads or pants, incontinence products and other adult care garments, medical garments, surgical pads and bandages, other personal care or health care garments, and the like without departing from the scope of the present disclosure.

The term “acquisition layer”, “acquisition/distribution layer”, or “acquisition and/or distribution layer” refers herein to a layer or layers capable of accepting and temporarily holding liquid body exudates to decelerate and diffuse a surge or gush of the liquid body exudates and to subsequently release the liquid body exudates therefrom into another layer or layers of the absorbent article (for example, absorbent layers).

The term “bonded” or “coupled” refers herein to the joining, adhering, connecting, attaching, or the like, of two elements. Two elements will be considered bonded or coupled together when they are joined, adhered, connected, attached, or the like, directly to one another or indirectly to one another, such as when each is directly bonded to intermediate elements. The bonding or coupling of one element to another can occur via continuous or intermittent bonds.

The term “carded web” refers herein to a web containing natural or synthetic staple length fibers typically having fiber lengths less than about 100 mm. Bales of staple fibers can undergo an opening process to separate the fibers which are then sent to a carding process which separates and combs the fibers to align them in the machine direction after which the fibers are deposited onto a moving wire for further processing. Such webs are usually subjected to some type of bonding process such as thermal bonding using heat and/or pressure. In addition to or in lieu thereof, the fibers may be subject to adhesive processes to bind the fibers together such as by the use of powder adhesives. The carded web may be subjected to fluid entangling, such as hydroentangling, to further intertwine the fibers and thereby improve the integrity of the carded web. Carded webs, due to the fiber alignment in the machine direction, once bonded, will typically have more machine direction strength than cross machine direction strength.

“Coform” refers to a non-woven composite material of air-formed matrix material comprising thermoplastic polymeric meltblown fibers such as, for example, microfibers having an average fiber diameter of less than about 10 microns, and a multiplicity of individualized absorbent fibers such as, for example, wood pulp fibers disposed throughout the matrix of polymer microfibers and engaging at least some of the microfibers to space the microfibers apart from each other. The absorbent fibers are interconnected by and held captive within the matrix of microfibers by mechanical entanglement of the microfibers with the absorbent fibers, the mechanical entanglement and interconnection of the microfibers and absorbent fibers alone forming a coherent integrated fibrous structure. These materials are prepared according to the descriptions in U.S. Pat. No. 4,100,324 to Anderson et al. U.S. Pat. No. 5,508,102 to Georger et al. and U.S. Pat. No. 5,385,775 to Wright.

“Elastomeric” refers to a material or composite which can be elongated by at least 50 percent of its relaxed length and which will recover, upon release of the applied force, at least 20 percent of its elongation. It is generally preferred that the elastomeric material or composite be capable of being elongated by at least 50 percent, more preferably by at least 100 percent, and still more preferably by at least 300 percent of its relaxed length and recover, upon release of an applied force, at least 50 percent of its elongation.

The term “film” refers herein to a thermoplastic film made using an extrusion and/or forming process, such as a cast film or blown film extrusion process. The term includes apertured films, slit films, and other porous films which constitute liquid transfer films, as well as films which do not transfer fluids, such as, but not limited to, barrier films, filled films, breathable films, and oriented films.

The term “gsm” refers herein to grams per square meter.

The term “hydrophilic” refers herein to fibers or the surfaces of fibers which are wetted by aqueous liquids in contact with the fibers. The degree of wetting of the materials can, in turn, be described in terms of the contact angles and the surface tensions of the liquids and materials involved. Equipment and techniques suitable for measuring the wettability of particular fiber materials or blends of fiber materials can be provided by Cahn SFA-222 Surface Force Analyzer System, or a substantially equivalent system. When measured with this system, fibers having contact angles less than 90 are designated “wettable” or hydrophilic, and fibers having contact angles greater than 90 are designated “nonwettable” or hydrophobic.

The term “liquid impermeable” refers herein to a layer or multi-layer laminate in which liquid body exudates, such as urine, will not pass through the layer or laminate, under ordinary use conditions, in a direction generally perpendicular to the plane of the layer or laminate at the point of liquid contact.

The term “liquid permeable” refers herein to any material that is not liquid impermeable.

The term “meltblown” refers herein to fibers formed by extruding a molten thermoplastic material through a plurality of fine, usually circular, die capillaries as molten threads or filaments into converging high velocity heated gas (e.g., air) streams which attenuate the filaments of molten thermoplastic material to reduce their diameter, which can be a microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al., which is incorporated herein by reference. Meltblown fibers are microfibers which may be continuous or discontinuous, are generally smaller than about 0.6 denier, and may be tacky and self-bonding when deposited onto a collecting surface.

The term “nonwoven” refers herein to materials and webs of material which are formed without the aid of a textile weaving or knitting process. The materials and webs of materials can have a structure of individual fibers, filaments, or threads (collectively referred to as “fibers”) which can be interlaid, but not in an identifiable manner as in a knitted fabric. Nonwoven materials or webs can be formed from many processes such as, but not limited to, meltblowing processes, spunbonding processes, carded web processes, etc.

The term “pliable” refers herein to materials which are compliant and which will readily conform to the general shape and contours of the wearer's body.

The term “spunbond” refers herein to small diameter fibers which are formed by extruding molten thermoplastic material as filaments from a plurality of fine capillaries of a spinnerette having a circular or other configuration, with the diameter of the extruded filaments then being rapidly reduced by a conventional process such as, for example, eductive drawing, and processes that are described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartmann, U.S. Pat. No. 3,502,538 to Peterson, and U.S. Pat. No. 3,542,615 to Dobo et al., each of which is incorporated herein in its entirety by reference. Spunbond fibers are generally continuous and often have average deniers larger than about 0.3, and in an embodiment, between about 0.6, about 5 and about 10 and about 15, about 20 and about 40. Spunbond fibers are generally not tacky when they are deposited on a collecting surface.

The term “superabsorbent” refers herein to a water-swellable, water-insoluble organic or inorganic material capable, under the most favorable conditions, of absorbing at least 15 times its weight and, in an embodiment, at least 30 times its weight, in an aqueous solution containing 0.9 weight percent sodium chloride. The superabsorbent materials can be natural, synthetic and modified natural polymers and materials. In addition, the superabsorbent materials can be inorganic materials, such as silica gels, or organic compounds, such as cross-linked polymers.

The term “super-majority” refers herein to a majority of at least 65%.

The term “thermoplastic” refers herein to a material which softens and which can be shaped when exposed to heat and which substantially returns to a non-softened condition when cooled.

The term “user” or “caregiver” refers herein to one who fits an absorbent article, such as, but not limited to, a diaper, diaper pant, training pant, youth pant, incontinent product, or other absorbent article about the wearer of one of these absorbent articles. A user and a wearer can be one and the same person.

### Absorbent Article:

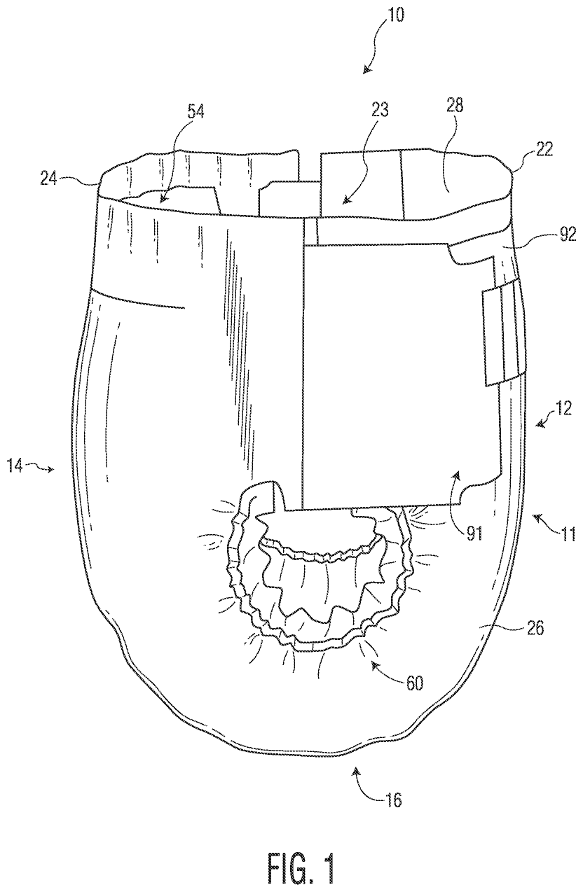

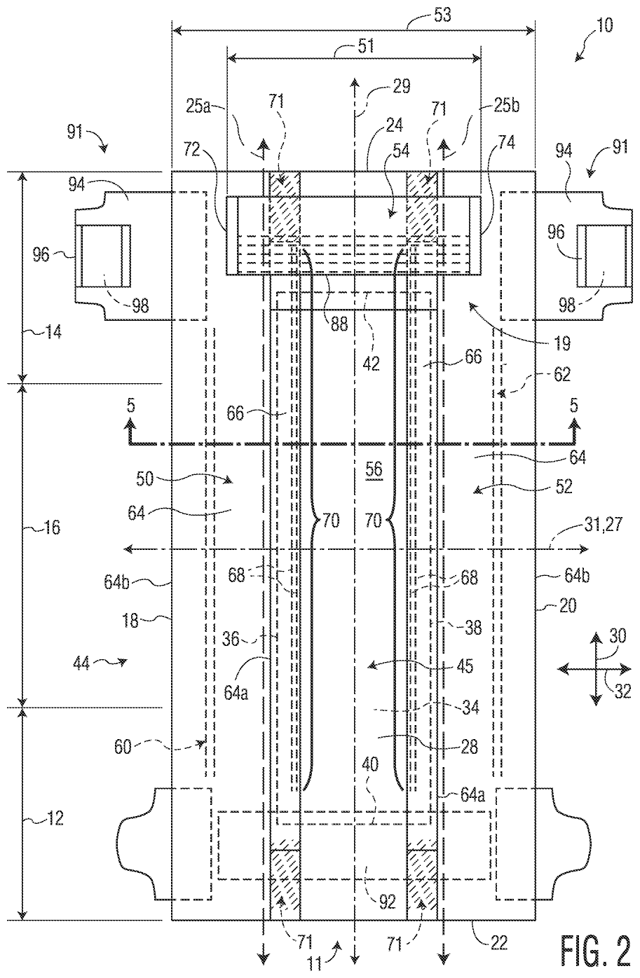

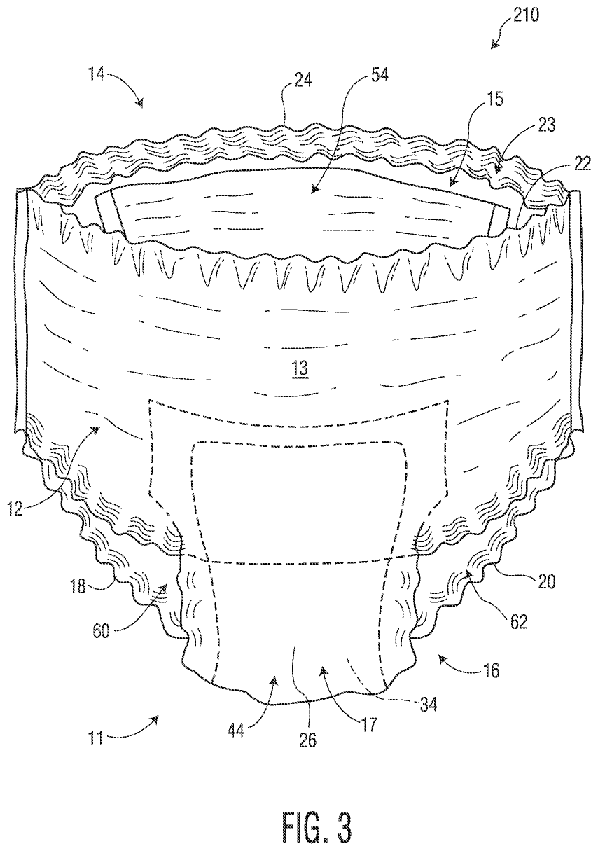

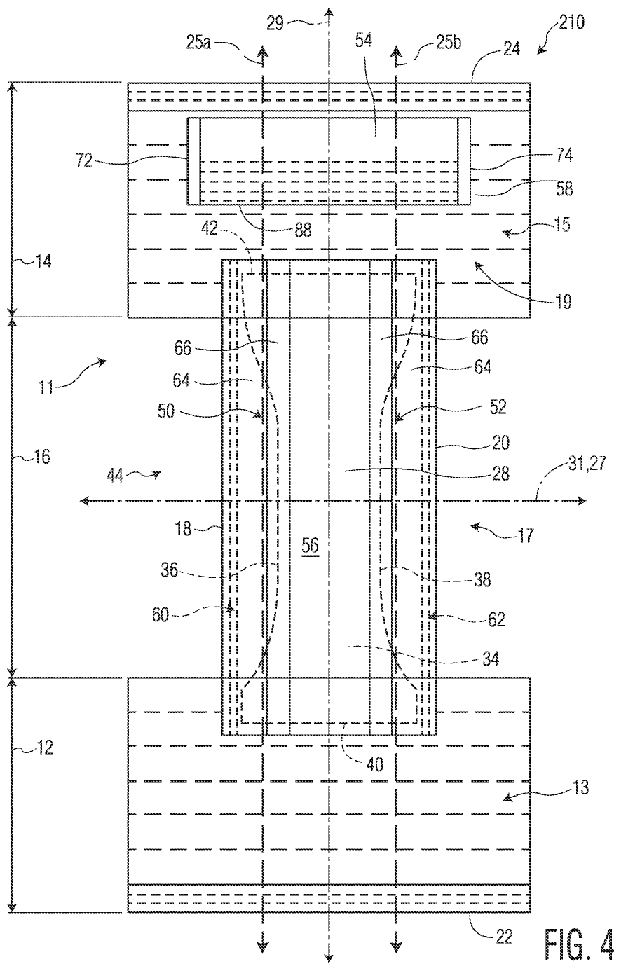

Referring to FIGS. 1-2, a non-limiting illustration of an absorbent article 10, for example a diaper, is illustrated. While the examples and illustrations described herein may generally apply to absorbent articles manufactured in the product longitudinal direction, which is hereinafter called the machine direction manufacturing of a product, it should be noted that one of ordinary skill in the art could apply the information herein to absorbent articles manufactured in the latitudinal direction of the product, which hereinafter is called the cross-machine direction manufacturing of a product, without departing from the spirit and scope of the disclosure. For example, the absorbent article 210 in FIGS. 3-4 provide an exemplary absorbent article 210 that can be manufactured in cross-machine direction manufacturing process.

The absorbent article 10 illustrated in FIGS. 1 and 2 and the absorbent article 210 illustrated in FIGS. 3 and 4 each generally comprise a chassis 11. The absorbent article 10, 210 has a front waist region 12, a rear waist region 14, and a crotch region 16 disposed between the front waist region 12 and the rear waist region 14 and interconnecting the front and rear waist regions, 12, 14, respectively. The front waist region 12 may be alternatively referred to as the front-end region, the rear waist region 14 as the rear-end region, and the crotch region 16 as the intermediate region. In the examples of FIGS. 3 and 4, a three-piece construction of an absorbent article 210 is depicted where the absorbent article 210 has a chassis 11 including a front waist panel 13 defining the front waist region 12, a rear waist panel 15 defining the rear waist region 14, and an absorbent panel 17 defining the crotch region 16 of the absorbent article 210. The absorbent panel 17 of FIGS. 3 and 4 extends between the front waist panel 13 and the rear waist panel 15. The absorbent panel 17 can overlap the front waist panel 13 and the rear waist panel 15 and be bonded to the front waist panel 13 and the rear waist panel 15 to define a three-piece construction. However, it is contemplated that an absorbent article can be manufactured in a cross-machine direction without being a three-piece construction garment—for example, constructed with a one-piece outer cover.

The absorbent article 10, 210 has a pair of longitudinal side edges 18, 20, and a pair of opposite waist edges, respectively designated front waist edge 22 and rear waist edge 24. The front waist region 12 can be contiguous with the front waist edge 22 and the rear waist region 14 can be contiguous with the rear waist edge 24. The longitudinal side edges 18, 20 can extend from the front waist edge 22 to the rear waist edge 24. The longitudinal side edges 18, 20 can extend in a direction parallel to the longitudinal direction 30 for their entire length, such as for the absorbent article 10 illustrated in FIGS. 1 and 2. In other embodiments, the longitudinal side edges 18, 20 can be curved between the front waist edge 22 and the rear waist edge 24. In the absorbent article 210 of FIGS. 3 and 4, the longitudinal side edges 18, 20 can include portions of the front waist panel 13, the absorbent panel 17, and the rear waist panel 15.

The front waist region 12 may generally correspond to the portion of the absorbent article 10, 210 that, when worn, is positioned at least in part on the front of the wearer while the rear waist region 14 may generally correspond to the portion of the absorbent article 10, 210 that, when worn, is positioned at least in part on the back of the wearer. The crotch region 16 of the absorbent article 10, 210 is disposed between the front waist region 12 and the rear waist region 14 and is the portion of the absorbent article 10, 210 that, when worn, is positioned between the legs of the wearer and can partially cover the lower torso of the wearer. The waist edges, 22 and 24, of the absorbent article 10, 210 are configured to encircle the waist of the wearer and together define a central waist opening 23 (as labeled in FIG. 1 and FIG. 3) for the waist of the wearer. Portions of the longitudinal side edges 18, 20 in the crotch region 16 can generally define leg openings for the legs of the wearer when the absorbent article 10, 210 is worn.

The example absorbent article 10, 210 of FIGS. 1-4 includes an outer cover 26 and a bodyside liner 28. The outer cover 26 and the bodyside liner 28 can form a portion of the chassis 11. In an example of absorbent articles contemplated by the present disclosure, the bodyside liner 28 can be bonded to the outer cover 26 in a superposed relation by any suitable means such as, but not limited to, adhesives, ultrasonic bonds, thermal bonds, pressure bonds, or other conventional techniques. The outer cover 26 defines a length in a longitudinal direction 30, and a width in the lateral direction 32, which, in the illustrated example of article 10 in FIGS. 1-2, can coincide with the length and width of the absorbent article 10. As illustrated in FIGS. 2 and 4, the absorbent article 10, 210 has a longitudinal axis 29 extending in the longitudinal direction 30 and a lateral axis 31 extending in the lateral direction 32.

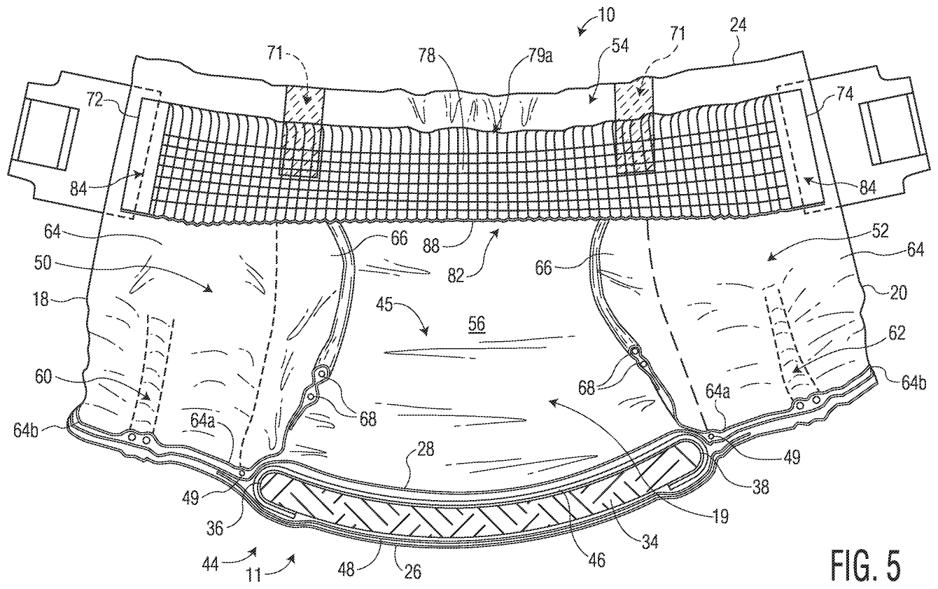

The chassis 11 includes an absorbent body 34. The absorbent body 34 is generally disposed between the outer cover 26 and the bodyside liner 28. The absorbent body 34 has longitudinal edges, 36 and 38, which, in at least some examples, can form portions of the longitudinal side edges, 18 and 20, respectively, of the absorbent article 10, 210. The absorbent body 34 has a first end edge 40 that is opposite a second end edge 42, respectively, which, in particular examples, form portions of the waist edges, 22 and 24, respectively, of the absorbent article 10. In some examples of the article 10, 210, the first end edge 40 is in the front waist region 12 and/or the second end edge 42 is in the rear waist region 14. In some examples of the article 10, 210, the absorbent body 34 has a length and width that are the same as or less than the length and width of the absorbent article 10, 210. The bodyside liner 28, the outer cover 26, and the absorbent body 34 can be considered to form part of an absorbent assembly 44 in some examples. In the absorbent article 210 of FIGS. 3 and 4, the absorbent panel 17 can form the absorbent assembly 44. In some examples of the article 10, 210, the absorbent assembly 44 can further include a fluid transfer layer 46 (as shown in FIG. 5) and/or a fluid acquisition layer (not shown) between the bodyside liner 28 and the fluid transfer layer 46 as is known in the art. The absorbent assembly 44 may also include a spacer layer 48 (as shown in FIG. 5) disposed between the absorbent body 34 and the outer cover 26.

The absorbent article 10, 210 are generally configured to contain and/or absorb fluid, solid, and semi-solid body exudates discharged from the wearer. In some examples, containment flaps 50, 52 can be configured to provide a barrier to the lateral flow of body exudates. To further enhance containment and/or absorption of body exudates, the absorbent article 10, 210 can suitably include a waist containment member 54. In some embodiments, the waist containment member 54 can be disposed in the rear waist region 14 of the absorbent article 10, 210. Although not depicted herein, it is contemplated that the waist containment member 54 can be additionally or alternatively disposed in the front waist region 12 of the absorbent article 10, 210.

The waist containment member 54 can be disposed on the body facing surface 19 of the chassis 11 to help contain and/or absorb body exudates. In some embodiments, such as in the absorbent articles 10 depicted in FIGS. 1 and 2, the waist containment member 54 can be disposed on the body facing surface 45 of the absorbent assembly 44. In some embodiments, the waist containment member 54 can be disposed on the body facing surface 56 of the bodyside liner 28. In some embodiments, such as in the absorbent article 210 depicted in FIGS. 3 and 4, the waist containment member 54 can be disposed on the body facing surface 58 of the rear waist panel 15.

The absorbent article 10, 210 can further include leg elastic members 60, 62 as are known to those skilled in the art. The leg elastic members 60, 62 can be attached to the outer cover 26 and/or the bodyside liner 28 along the opposite longitudinal side edges, 18 and 20, and positioned in the crotch region 16 of the absorbent article 10, 210. The leg elastic members 60, 62 can be parallel to the longitudinal axis 29 as shown in FIGS. 2 and 4 or can be curved as is known in the art. The leg elastic members 60, 62 can be elastomeric and can provide elasticized leg cuffs.

In some embodiments, the absorbent article 10, 210 can further include longitudinal extending fold lines 25a, 25b, as shown in FIGS. 2 and 4. The first longitudinal extending fold line 25a can be on one side of the longitudinal axis 29 of the absorbent article 10, 210 and the second longitudinal extending fold line 25b can be on an opposite side of the longitudinal axis 29. In some embodiments, the longitudinal extending fold lines 25a, 25b can be generally parallel to the longitudinal axis 29 of the absorbent article 10, 210. In some embodiments, the absorbent article 10, 210 can further include a lateral extending fold line 27. The lateral extending fold line 27 can be parallel to and located at the lateral axis 31 of the absorbent article 10, 210 in some embodiments.

Additional details regarding each of these elements of the absorbent article 10, 210 described herein can be found below and with reference to the Figures.

### Outer Cover:

The outer cover 26 and/or portions thereof can be breathable and/or liquid impermeable. The outer cover 26 and/or portions thereof can be elastic, stretchable, or non-stretchable. The outer cover 26 may be constructed of a single layer, multiple layers, laminates, spunbond fabrics, films, meltblown fabrics, elastic netting, microporous webs, bonded-carded webs or foams provided by elastomeric or polymeric materials. In an embodiment, for example, the outer cover 26 can be constructed of a microporous polymeric film, such as polyethylene or polypropylene.

In an embodiment, the outer cover 26 can be a single layer of a liquid impermeable material, such as a polymeric film. In an embodiment, the outer cover 26 can be suitably stretchable, and more suitably elastic, in at least the lateral direction 32 of the absorbent article 10, 210. In an embodiment, the outer cover 26 can be stretchable, and more suitably elastic, in both the lateral 32 and the longitudinal 30 directions. In an embodiment, the outer cover 26 can be a multi-layered laminate in which at least one of the layers is liquid impermeable. In some embodiments, the outer cover 26 can be a two-layer construction, including an outer layer (not shown) and an inner layer (not shown) which can be bonded together such as by a laminate adhesive. Suitable laminate adhesives can be applied continuously or intermittently as beads, a spray, parallel swirls, or the like, but it is to be understood that the inner layer can be bonded to the outer layer by other bonding methods, including, but not limited to, ultrasonic bonds, thermal bonds, pressure bonds, or the like.

The outer layer of the outer cover 26 can be any suitable material and may be one that provides a generally cloth-like texture or appearance to the wearer. An example of such material can be a 100% polypropylene bonded-carded web with a diamond bond pattern available from Sandler A.G., Germany, such as 30 gsm Sawabond 4185® or equivalent. Another example of material suitable for use as an outer layer of an outer cover 26 can be a 20 gsm spunbond polypropylene non-woven web. The outer layer may also be constructed of the same materials from which the bodyside liner 28 can be constructed as described herein.

The liquid impermeable inner layer of the outer cover 26 (or the liquid impermeable outer cover 26 where the outer cover 26 is of a single-layer construction) can be either vapor permeable (i.e., “breathable”) or vapor impermeable. The liquid impermeable inner layer (or the liquid impermeable outer cover 26 where the outer cover 26 is of a single-layer construction) can be manufactured from a thin plastic film. The liquid impermeable inner layer (or the liquid impermeable outer cover 26 where the outer cover 26 is of a single-layer construction) can inhibit liquid body exudates from leaking out of the absorbent article 10, 210 and wetting articles, such as bed sheets and clothing, as well as the wearer and caregiver.

In some embodiments, where the outer cover 26 is of a single layer construction, it can be embossed and/or matte finished to provide a more cloth-like texture or appearance. The outer cover 26 can permit vapors to escape from the absorbent article 10 while preventing liquids from passing through. A suitable liquid impermeable, vapor permeable material can be composed of a microporous polymer film or a non-woven material which has been coated or otherwise treated to impart a desired level of liquid impermeability.

### Bodyside Liner:

The bodyside liner 28 of the absorbent article 10, 210 can overlay the absorbent body 34 and the outer cover 26 and can isolate the wearer's skin from liquid waste retained by the absorbent body 34. In various embodiments, a fluid transfer layer 46 can be positioned between the bodyside liner 28 and the absorbent body 34. In various embodiments, an acquisition layer (not shown) can be positioned between the bodyside liner 28 and the absorbent body 34 or a fluid transfer layer 46, if present. In various embodiments, the bodyside liner 28 can be bonded to the acquisition layer, or to the fluid transfer layer 46 if no acquisition layer is present, via adhesive and/or by a point fusion bonding. The point fusion bonding may be selected from ultrasonic, thermal, pressure bonding, and combinations thereof.

In an embodiment, the bodyside liner 28 can extend beyond the absorbent body 34 and/or a fluid transfer layer 46, if present, and/or an acquisition layer, if present, and/or a spacer layer 48, if present, to overlay a portion of the outer cover 26 and can be bonded thereto by any method deemed suitable, such as, for example, by being bonded thereto by adhesive, to substantially enclose the absorbent body 34 between the outer cover 26 and the bodyside liner 28. The bodyside liner 28 may be narrower than the outer cover 26. However, in other embodiments, the bodyside liner 28 and the outer cover 26 may be of the same dimensions in width and length. In other embodiments, the bodyside liner 28 can be of greater width than the outer cover 26. It is also contemplated that the bodyside liner 28 may not extend beyond the absorbent body 34 and/or may not be secured to the outer cover 26. In some embodiments, the bodyside liner 28 can wrap at least a portion of the absorbent body 34, including wrapping around both longitudinal edges 36, 38 of the absorbent body 34, and/or one or more of the end edges 40, 42. It is further contemplated that the bodyside liner 28 may be composed of more than one segment of material. The bodyside liner 28 can be of different shapes, including rectangular, hourglass, or any other shape. The bodyside liner 28 can be suitably compliant, soft feeling, and non-irritating to the wearer's skin and can be the same as or less hydrophilic than the absorbent body 34 to permit body exudates to readily penetrate through to the absorbent body 34 and provide a relatively dry surface to the wearer.

The bodyside liner 28 can be manufactured from a wide selection of materials, such as synthetic fibers (for example, polyester or polypropylene fibers), natural fibers (for example, wood or cotton fibers), a combination of natural and synthetic fibers, porous foams, reticulated foams, apertured plastic films, or the like. Examples of suitable materials include, but are not limited to, rayon, wood, cotton, polyester, polypropylene, polyethylene, nylon, or other heat-bondable fibers, polyolefins, such as, but not limited to, copolymers of polypropylene and polyethylene, linear low-density polyethylene, and aliphatic esters such as polylactic acid, finely perforated film webs, net materials, and the like, as well as combinations thereof.

Various woven and non-woven fabrics can be used for the bodyside liner 28. The bodyside liner 28 can include a woven fabric, a nonwoven fabric, a polymer film, a film-fabric laminate or the like, as well as combinations thereof. Examples of a nonwoven fabric can include spunbond fabric, meltblown fabric, coform fabric, carded web, bonded-carded web, bicomponent spunbond fabric, spunlace, or the like, as well as combinations thereof. The bodyside liner 28 need not be a unitary layer structure, and thus, can include more than one layer of fabrics, films, and/or webs, as well as combinations thereof. For example, the bodyside liner 28 can include a support layer and a projection layer that can be hydroentagled. The projection layer can include hollow projections, such as those disclosed in U.S. Pat. No. 9,474,660 to Kirby, Scott S. C. et al.

For example, the bodyside liner 28 can be composed of a meltblown or spunbond web of polyolefin fibers. Alternatively, the bodyside liner 28 can be a bonded-carded web composed of natural and/or synthetic fibers. The bodyside liner 28 can be composed of a substantially hydrophobic material, and the hydrophobic material can, optionally, be treated with a surfactant or otherwise processed to impart a desired level of wettability and hydrophilicity. The surfactant can be applied by any conventional means, such as spraying, printing, brush coating or the like. The surfactant can be applied to the entire bodyside liner 28 or it can be selectively applied to particular sections of the bodyside liner 28.

In an embodiment, a bodyside liner 28 can be constructed of a non-woven bicomponent web. The non-woven bicomponent web can be a spunbonded bicomponent web, or a bonded-carded bicomponent web. An example of a bicomponent staple fiber includes a polyethylene/polypropylene bicomponent fiber. In this particular bicomponent fiber, the polypropylene forms the core and the polyethylene forms the sheath of the fiber. Fibers having other orientations, such as multi-lobe, side-by-side, end-to-end may be used without departing from the scope of this disclosure. In an embodiment, a bodyside liner 28 can be a spunbond substrate with a basis weight from about 10 or about 12 to about 15 or about 20 gsm. In an embodiment, a bodyside liner 28 can be a 12 gsm spunbond-meltblown-spunbond substrate having 10% meltblown content applied between the two spunbond layers.

Although the outer cover 26 and bodyside liner 28 can include elastomeric materials, it is contemplated that the outer cover 26 and the bodyside liner 28 can be composed of materials which are generally non-elastomeric. In an embodiment, the bodyside liner 28 can be stretchable, and more suitably elastic. In an embodiment, the bodyside liner 28 can be suitably stretchable and more suitably elastic in at least the lateral or circumferential direction of the absorbent article 10, 210. In other aspects, the bodyside liner 28 can be stretchable, and more suitably elastic, in both the lateral and the longitudinal directions 32, 30, respectively.

### Containment Flaps:

In an embodiment, the absorbent article 10, 210 can include a pair of containment flaps 50, 52. The containment flaps 50, 52 can be formed separately from the absorbent chassis 11 and attached to the chassis 11 or can be formed integral to the chassis 11. In some embodiments, the containment flaps 50, 52 can be secured to the chassis 11 of the absorbent article 10, 210 in a generally parallel, spaced relation with each other laterally inward of the leg openings to provide a barrier against the flow of body exudates. One containment flap 50 can be on a first side of the longitudinal axis 29 and the other containment flap 52 can be on a second side of the longitudinal axis 29. In an embodiment, the containment flaps 50, 52 can extend generally in a longitudinal direction 30 from the front waist region 12 of the absorbent article 10, through the crotch region 16 to the rear waist region 14 of the absorbent article 10. In some embodiments, the containment flaps 50, 52 can extend in a direction substantially parallel to the longitudinal axis 29 of the absorbent article 10, 210, however, in other embodiments, the containment flaps 50, 52 can be curved, as is known in the art. In other embodiments, such as the absorbent article 210 in FIGS. 3 and 4, the containment flaps 50, 52 can be disposed on the absorbent panel 17 in the crotch region 16.

In embodiments where the containment flaps 50, 52 are coupled to the chassis 11, the containment flaps 50, 52 can be bonded to the bodyside liner 28 with a barrier adhesive 49, as shown in FIG. 5. Alternatively, the containment flaps 50, 52 can be bonded to the outer cover 26 with a barrier adhesive 49, or to the spacer layer 48. Of course, the containment flaps 50, 52 can be bonded to other components of the chassis 11 and can be bonded with other suitable means other than a barrier adhesive 49. The containment flaps 50, 52 can be constructed of a fibrous material which can be similar to the material forming the bodyside liner 28. Other conventional materials, such as polymer films, can also be employed.

The containment flaps 50, 52 can each include a base portion 64 and a projection portion 66. The base portion 64 can be bonded to the chassis 11, for example, to the bodyside liner 28 or the outer cover 26 as mentioned above. The base portion 64 can include a proximal end 64a and a distal end 64b. The projection portion 66 can be separated from the base portion 64 at the proximal end 64a of the base portion 64. As used in this context, the projection portion 66 is separated from the base portion 64 at the proximal end 64a of the base portion 64 in that the proximal end 64a of the base portion 64 defines a transition between the projection portion 66 and the base portion 64. The proximal end 64a of the base portion 64 can be located near the barrier adhesive 49. In some embodiments, the distal ends 64b of the base portion 64 can laterally extend to the respective longitudinal side edges 18, 20 of the absorbent article 10, 210. In other embodiments, the distal ends 64b of the base portion 64 can end laterally inward of the respective longitudinal side edges 18, 20 of the absorbent article 10, 210. The containment flaps 50, 52 can also each include a projection portion 66 that is configured to extend away from the body facing surface 19 of the chassis 11 at least in the crotch region 16 when the absorbent article 10, 210 is in a relaxed configuration, as illustrated in FIG. 5. The containment flaps 50, 52 can include a tack-down region 71 in either or both of the front waist region 12 and the rear waist region 14 where the projection portion 66 is coupled to the body facing surface 19 of the chassis 11.

It is contemplated that the containment flaps 50, 52 can be of various configurations and shapes, and can be constructed by various methods. For example, the containment flaps 50, 52 of FIG. 5 depict a vertical containment flap 50, 52 with a tack-down region 71 in both the front and rear waist regions 12, 14 where the projection portion 66 of each containment flap 50, 52 is tacked down to the bodyside liner 28 towards or away from the longitudinal axis 29 of the absorbent article 10, 210. However, the containment flaps 50, 52 can include a tack-down region 71 where the projection portion 66 of each of the containment flaps 50, 52 is folded back upon itself and coupled to itself and the bodyside liner 28 in a “C-shape” configuration, as is known in the art and described in U.S. Pat. No. 5,895,382 to Robert L. Popp et al. As yet another alternative, it is contemplated that the containment flaps 50, 52 could be constructed in a “T-shape” configuration, such as described in U.S. Pat. No. 9,259,362 by Robert L. Popp et al. Such a configuration can also include a tack-down region 71 in either or both of the front and rear waist regions 12, 14, respectively. Of course, other configurations of containment flaps 50, 52 can be used in the absorbent article 10, 210 and still remain within the scope of this disclosure.

The containment flaps 50, 52 can include one or more flap elastic members 68, such as the two flap elastic strands depicted in FIG. 5. Suitable elastomeric materials for the flap elastic members 68 can include sheets, strands or ribbons of natural rubber, synthetic rubber, or thermoplastic elastomeric materials. Of course, while two elastic members 68 are shown in each containment flap 50, 52, it is contemplated that the containment flaps 50, 52 can be configured with one or three or more elastic members 68. Alternatively or additionally, the containment flaps 50, 52 can be composed of a material exhibiting elastomeric properties itself.

The flap elastic members 68, as illustrated in FIG. 5, can have two strands of elastomeric material extending longitudinally in the projection portion 66 of the containment flaps 50, 52, in generally parallel, spaced relation with each other. The elastic members 68 can be within the containment flaps 50, 52 while in an elastically contractible condition such that contraction of the strands gathers and shortens the projection portions 66 of the containment flaps 50, 52 in the longitudinal direction 30. As a result, the elastic members 68 can bias the projection portions 66 of the containment flaps 50, 52 to extend away from the body facing surface 45 of the absorbent assembly 44 in a generally upright orientation of the containment flaps 50, 52, especially in the crotch region 16 of the absorbent article 10, 210, when the absorbent article 10 is in a relaxed configuration.

During manufacture of the containment flaps 50, 52 at least a portion of the elastic members 68 can be bonded to the containment flaps 50, 52 while the elastic members 68 are elongated. The percent elongation of the elastic members 68 can be, for example, about 110% to about 350%. In one embodiment, the elastic members 68 can be coated with adhesive while elongated to a specified length prior to attaching to the elastic members 68 to the containment flaps 50, 52. In a stretched condition, the length of the elastic members 68 which have adhesive coupled thereto can provide an active flap elastic region 70 in the containment flaps 50, 52, as labeled in FIG. 2, which will gather upon relaxation of the absorbent article 10. The active flap elastic region 70 of containment flaps 50, 52 can be of a longitudinal length that is less than the length of the absorbent article 10, 210. In this exemplary method of bonding the elastic members 68 to the containment flaps 50, 52, the portion of the elastic members 68 not coated with adhesive, will retract after the elastic members 68 and the absorbent article 10 are cut in manufacturing to form an individual absorbent article 10. As noted above, the relaxing of the elastic members 68 in the active flap elastic region 70 when the absorbent article 10, 210 is in a relaxed condition can cause each containment flap 50, 52 to gather and cause the projection portion 66 of each containment flap 50, 52 to extend away from the body facing surface 19 of the chassis 11 (e.g., the body facing surface 45 of the absorbent assembly 44 or the body facing surface 56 of the bodyside liner 28), as depicted in FIG. 5.

Of course, the elastic members 68 can be bonded to the containment flaps 50, 52 in various other ways as known by those of skill in the art to provide an active flap elastic region 70, which is within the scope of this disclosure. Additionally, the active flap elastic regions 70 can be shorter or longer than depicted herein, including extending to the front waist edge 22 and the rear waist edge 24, and still be within the scope of this disclosure.

### Leg Elastics:

Leg elastic members 60, 62 can be secured to the outer cover 26, such as by being bonded thereto by laminate adhesive, generally laterally inward of the longitudinal side edges, 18 and 20, of the absorbent article 10, 210. The leg elastic members 60, 62 can form elasticized leg cuffs that further help to contain body exudates. In an embodiment, the leg elastic members 60, 62 may be disposed between inner and outer layers (not shown) of the outer cover 26 or between other layers of the absorbent article 10, for example, between the base portion 64 of each containment flap 50, 52 and the bodyside liner 28 as depicted in FIG. 5, between the base portion 64 of each containment flap 50, 52 and the outer cover 26, or between the bodyside liner 28 and the outer cover 26. The leg elastic members 60, 62 can be one or more elastic components near each longitudinal side edge 18, 20. For example, the leg elastic members 60, 62 as illustrated herein each include two elastic strands. A wide variety of elastomeric materials may be used for the leg elastic members 60, 62.

Suitable elastomeric materials can include sheets, strands or ribbons of natural rubber, synthetic rubber, or thermoplastic elastomeric materials. The elastomeric materials can be stretched and secured to a substrate, secured to a gathered substrate, or secured to a substrate and then elasticized or shrunk, for example, with the application of heat, such that the elastic retractive forces are imparted to the substrate. Additionally, it is contemplated that the leg elastic members 60, 62 can be formed with the containment flaps 50, 52, and then attached to the chassis 11 in some embodiments. Of course, the leg elastic members 60, 62 can be omitted from the absorbent article 10, 210 without departing from the scope of this disclosure.

### Waist Containment Member:

In an embodiment, the absorbent article 10, 210 can have one or more waist containment members 54. The waist containment member(s) 54 can be disposed in the rear waist region 14 as illustrated in FIGS. 1-5. In general, the waist containment member 54 can help contain and/or absorb body exudates, especially low viscosity fecal matter, and as such, can be preferred to be in the rear waist region 14. In some embodiments, the absorbent article 10, 210 can have a waist containment member 54 disposed in the front waist region 12. A waist containment member 54 in the front waist region 12 can help contain and/or absorb body exudates, such as urine, in the front waist region 12. Although not as prevalent as in the rear waist region 14, in some circumstances, fecal material may also spread to the front waist region 12, and thus, a waist containment member 54 disposed in the front waist region 12 can help contain and/or absorb body exudates as well. In other embodiments, the absorbent article 10, 210 can have a waist containment member 54 in both the rear waist region 14 and the front waist region 12.

The waist containment member 54 can be disposed on the body facing surface 45 of the absorbent assembly 44. In some embodiments, such as in embodiments illustrated in FIGS. 1-2 and 5, the waist containment member 54 can be disposed on the body facing surface 56 of the bodyside liner 28. However, in some embodiments, such as the absorbent article 210 in FIG. 4, the waist containment member 54 can be disposed on a body facing surface 58 of the rear waist panel 15.

The waist containment member 54 can include a first longitudinal side edge 72 and a second longitudinal side edge 74. The first longitudinal side edge 72 can be opposite from the second longitudinal side edge 74. The distance between the first longitudinal side edge 72 and the second longitudinal side edge 74 can define a width 51 of the waist containment member 54 in the lateral direction 32, as shown in FIG. 2.

As illustrated in FIGS. 2 and 5, the waist containment member 54 can be configured such that the first longitudinal side edge 72 can be disposed laterally outward of the proximal end 64a of the base portion 64 of the containment flap 50. Similarly, the waist containment member 54 can be configured such that the second longitudinal side edge 74 can be disposed laterally outward of the proximal end 64a of the base portion 64 of the containment flap 52. The waist containment member 54 can be configured such that the width 51 of the waist containment member 54 can be greater than a lateral distance between longitudinal extending fold lines 25a, 25b, as shown in FIGS. 2 and 4.

The waist containment member 54 can also include a proximal portion (not shown) and a distal portion 78. The proximal portion can be coupled to the body facing surface 19 of chassis 11 (e.g., the body facing surface 45 of the absorbent assembly 44 or the body facing surface 56 of the bodyside liner 28) whereas the distal portion 78 of the waist containment member 54 can be free to move with respect to the chassis 11 and the absorbent assembly 44 when the absorbent article 10, 210 is in the relaxed configuration, such as shown in FIG. 5. When the waist containment member 54 is in a relaxed configuration, the distal portion 78 extends away from the chassis 11 and absorbent assembly 44 in a vertical direction, which is perpendicular to the plane defined by the longitudinal axis 29 and the lateral axis 31. A fold 79a can separate the proximal portion from the distal portion 78 of the waist containment member 54. As used in this context, the fold 79a separates the proximal portion from the distal portion 78 in that the fold 79a defines a transition between the proximal portion and the distal portion 78.

In some embodiments, the proximal portion of the waist containment member 54 can be coupled to the body facing surface 56 of the bodyside liner 28. In other embodiments, the proximal portion of the waist containment member 54 can be coupled to the body facing surface 58 of the rear waist panel 15. The proximal portion can be coupled to the body facing surface 45 by an adhesive, by pressure bonding, by ultrasonic bonding, by thermal bonding, and combinations thereof.

Because the distal portion 78 of the waist containment member 54 can freely move with respect to the absorbent assembly 44 when the absorbent article 10, 210 is in the relaxed configuration, the distal portion 78 can help provide a containment pocket 82 when the absorbent article 10, 210 is in the relaxed configuration. The containment pocket 82 can help provide a barrier to contain and/or can help absorb body exudates. The containment pocket 82 can be especially beneficial for containing and/or absorbing low viscosity fecal matter, which can be prevalent in younger children. The first longitudinal side edge 72 can be disposed laterally outward of the proximal end 64a of the base portion 64 of the containment flap 50, and thus, the containment pocket 82 can extend laterally outward of the proximal end 64a of the containment flap 50. Similarly, the second longitudinal side edge 74 can be disposed laterally outward of the proximal end 64a of the base portion 64 of the containment flap 52 and the containment pocket 82 can extend laterally outward of the proximal end 64a of the containment flap 52. Such a configuration provides waist containment member 54 with a wide containment pocket 82 to contain and/or absorb body exudates.

To help prevent lateral flow of body exudates that are contained by the containment pocket 82 of the waist containment member 54, the distal portion 78 of the waist containment member 54 can be bonded to the proximal portion of the waist containment member 54 and/or the body facing surface 19 of the chassis 11 near the first and second longitudinal side edges 72, 74, respectively. For example, FIG. 5 depicts tack-down regions 84 where the distal portion 78 of the waist containment member 54 can be bonded to the proximal portion of the waist containment member 54 and/or the body facing surface 19 of the chassis 11.

In preferred embodiments, the waist containment member 54 can include at least one elastic member and even more elastic members in further embodiments. Generally, the elastic member can span substantially from the first longitudinal side edge 72 to the second longitudinal side edge 74 of the waist containment member 54. The elastic member can be disposed in the distal portion 78 of the waist containment member 54, and preferably, is located near a free edge 88 of the distal portion 78 of the waist containment member 54.

A wide variety of elastomeric materials may be used for the elastic member(s) in the waist containment member 54. Suitable elastomeric materials can include sheets, strands or ribbons of natural rubber, synthetic rubber, elastic foams, or thermoplastic elastomeric materials (e.g., films). The elastomeric materials can be stretched and secured to a substrate forming the waist containment member 54, secured to a gathered substrate, or secured to a substrate and then elasticized or shrunk, for example, with the application of heat, such that the elastic retractive forces are imparted to the substrate forming the waist containment member 54.

The waist containment member 54 can be disposed to be coupled to the chassis 11 by being placed either over the containment flaps 50, 52 or under the containment flaps 50, 52. More specifically, the waist containment member 54 can be disposed on the body facing surface 19 of the chassis 11 such that the proximal portion of the waist containment member 54 is disposed over the base portion 64 of the first and the second containment flaps 50, 52, respectively. Alternatively, the waist containment member 54 can be disposed on the body facing surface 19 of the chassis 11 such that the proximal portion of the waist containment member 54 is disposed under the base portion 64 of the first and the second containment flaps 50, 52, respectively. Both configurations can provide advantages to the functioning of the waist containment member 54 to contain and/or absorb body exudates.

Where the proximal portion of the waist containment member 54 is disposed over the base portion 64 of the containment flaps 50, 52, the containment flaps 50, 52 can have an active flap elastic region 70 that longitudinally overlaps with the distal portion 78 of the waist containment member 54 when the absorbent article 10 is in the stretched, laid flat configuration, such as illustrated in FIG. 2. Additionally or alternatively, the tack-down region 71 may not extend from the rear waist edge 24 to the free edge 88 of the distal portion 78 of the waist containment member 54, such as illustrated in FIG. 2.

Where the proximal portion of the waist containment member 54 is disposed under the base portion 64 of the containment flaps 50, 52, the tack-down region 71 of the projection portion 66 of each of the containment flaps 50, 52 may longitudinally overlap with the distal portion 78 of the waist containment member 54. In some of these embodiments, the tack-down region 71 of projection portion 66 of each of the containment flaps 50, 52 can extend to the free edge 88 of the waist containment member 54 to further assist in containing exudates to the containment pocket 82 created by the waist containment member 54.

The waist containment member 54 can be comprised of a variety of materials. In a preferred embodiment, the waist containment member 54 can be comprised of a spunbond-meltblown-spunbond (“SMS”) material. However it is contemplated that the waist containment member 54 can be comprised of other materials including, but not limited to, a spunbond material, a spunbond-film (“SF”) or spunbond-film-spunbond (“SFS”), a bonded carded web (“BCW”) such as thermal point bonded BCW, a through-air bonded carded web (TABCW), or any non-woven material. In some embodiments, the waist containment member 54 can be comprised of a laminate of more than one of these exemplary materials, or other materials. In some embodiments, the waist containment member 54 can be comprised of a liquid impermeable material. In some embodiments, the waist containment member 54 can be comprised of a material coated with a hydrophobic coating. The basis weight of the material forming the waist containment member 54 can vary, however, in a preferred embodiment, the basis weight can be between about 8 gsm to about 120 gsm, not including the elastic members 86 in the waist containment member 54. More preferably, the basis weight of the material comprising the waist containment member 54 can be between about 10 gsm to about 40 gsm, and even more preferably, between about 15 gsm to about 25 gsm.

### Fastening System:

In an embodiment, the absorbent article 10 can include a fastening system. The fastening system can include one or more back fasteners 91 and one or more front fasteners 92. The embodiments shown in FIGS. 1 and 2 depict embodiments with one front fastener 92. Portions of the fastening system may be included in the front waist region 12, rear waist region 14, or both.

The fastening system can be configured to secure the absorbent article 10 the waist of the wearer in a fastened condition as shown in FIG. 1 and help maintain the absorbent article 10 in place during use. In an embodiment, the back fasteners 91 can include one or more materials bonded together to form a composite ear as is known in the art. For example, the composite fastener may be composed of a stretch component 94, a nonwoven carrier or hook base 96, and a fastening component 98, as labeled in FIG. 2. As shown in FIG. 5, in some embodiments the waist containment member 54 can extend to the back fasteners 91. In some embodiments, the waist containment member 54 can be coupled to the stretch component 94 of the back fasteners 91, either directly or indirectly. In some embodiments, the waist containment member 54 can extend to the longitudinal side edges 18, 20 of the absorbent article 10, 210.

### Absorbent Body and Method of Manufacture:

The absorbent body 34 can be suitably constructed to be generally compressible, conformable, pliable, non-irritating to the wearer's skin and capable of absorbing and retaining liquid body exudates. The absorbent body 34 can be manufactured in a wide variety of sizes and shapes (for example, rectangular, trapezoidal, T-shape, I-shape, hourglass shape, etc.) and from a wide variety of materials. The size and the absorbent capacity of the absorbent body 34 should be compatible with the size of the intended wearer (infants to adults) and the liquid loading imparted by the intended use of the absorbent article 10, 210. The absorbent body 34 can have a length and width that can be less than or equal to the length and width of the absorbent article 10, 210.

The absorbent body 34 is generally composed of absorbent material, such as fibrous absorbent material and/or, superabsorbent material (“SAM” herein), and can include further materials such as binder materials, surfactants, selected hydrophobic and hydrophilic materials, pigments, lotions, odor control agents or the like, as well as combinations thereof. According to some examples, the absorbent body 34 is a matrix of cellulosic fluff and superabsorbent material. In other examples, the absorbent material of the absorbent body 34 comprises only superabsorbent material. The absorbent body 34 may be constructed of a single layer of materials, or in the alternative, may be constructed of two or more layers of materials.

When composed at least partially of fibrous material, various types of wettable, hydrophilic fibers can be used in the absorbent body 34. Examples of suitable fibers include natural fibers, cellulosic fibers, synthetic fibers composed of cellulose or cellulose derivatives, such as rayon fibers; inorganic fibers composed of an inherently wettable material, such as glass fibers; synthetic fibers made from inherently wettable thermoplastic polymers, such as particular polyester or polyamide fibers, or composed of nonwettable thermoplastic polymers, such as polyolefin fibers which have been hydrophilized by suitable means. The fibers may be hydrophilized, for example, by treatment with a surfactant, treatment with silica, treatment with a material which has a suitable hydrophilic moiety and is not readily removed from the fiber, or by sheathing the nonwettable, hydrophobic fiber with a hydrophilic polymer during or after formation of the fiber.

When composed at least partially of superabsorbent materials, such superabsorbent materials can be selected from natural, synthetic, and modified natural polymers and materials. The superabsorbent materials can be inorganic materials, such as silica gels, or organic compounds, such as cross-linked polymers.

If a spacer layer 48 is present, the absorbent body 34 can be disposed on the spacer layer 48 and superposed over the outer cover 26. The spacer layer 48 can be a nonwoven web material or a composite of fibrous material, and may be bonded to the outer cover 26, for example, by adhesive. In some embodiments, a spacer layer 48 may not be present and the absorbent body 34 can directly contact the outer cover 26 and can be directly bonded to the outer cover 26. However, it is to be understood that the absorbent body 34 may be in contact with, and not bonded with, the outer cover 26 and remain within the scope of this disclosure. In an embodiment, the outer cover 26 can be composed of a single layer and the absorbent body 34 can be in contact with the singer layer of the outer cover 26. In some embodiments, at least a portion of a layer, such as but not limited to, a fluid transfer layer 46 and/or a spacer layer 48, can be positioned between the absorbent body 34 and the outer cover 26, such as illustrated in FIG. 5. The absorbent body 34 can be bonded to the fluid transfer layer 46 and/or the spacer layer 48.

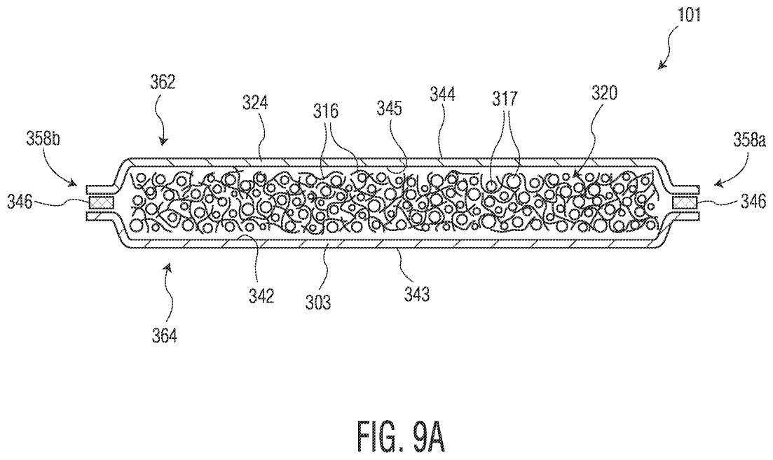

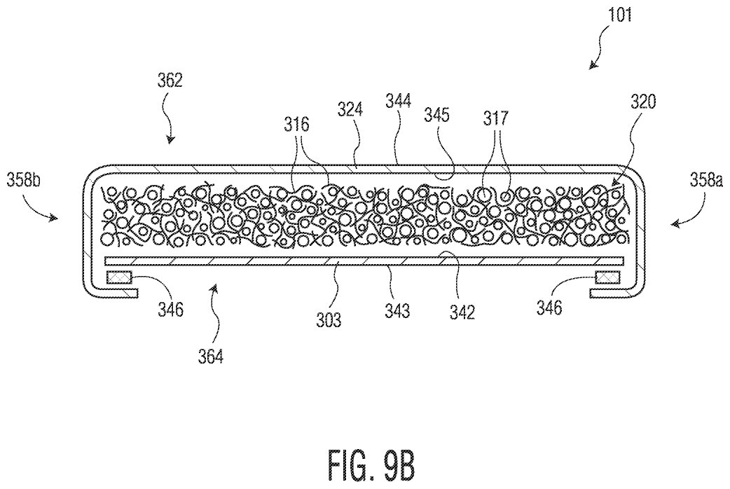

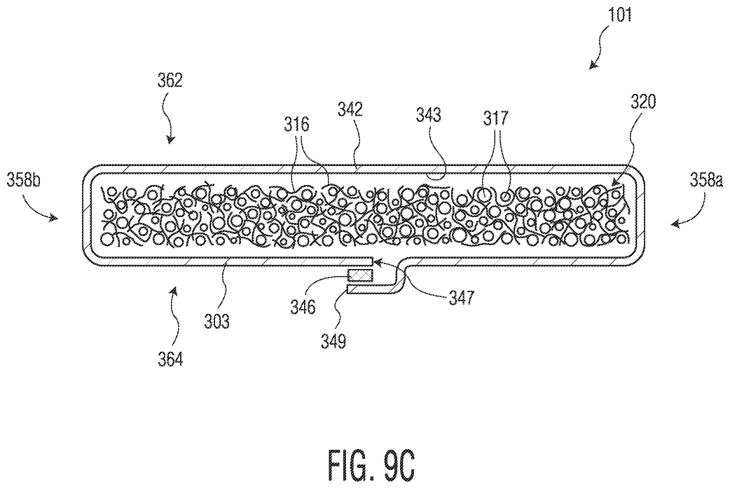

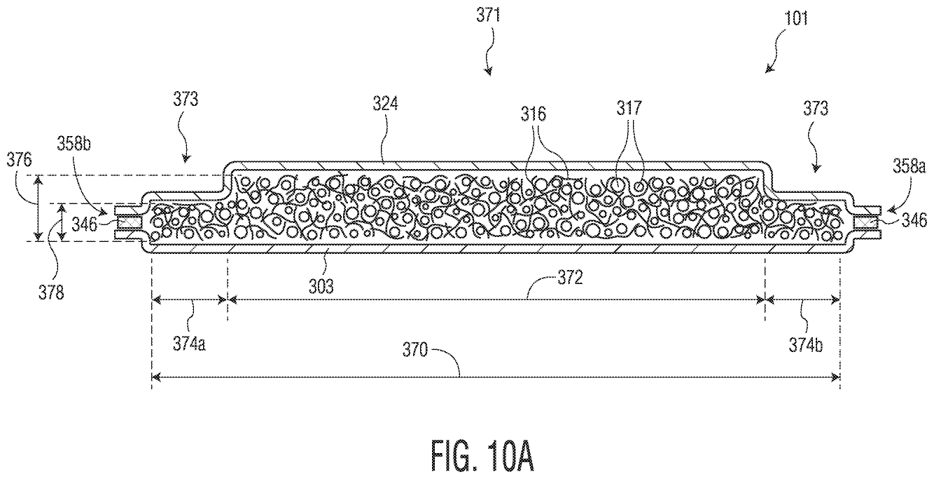

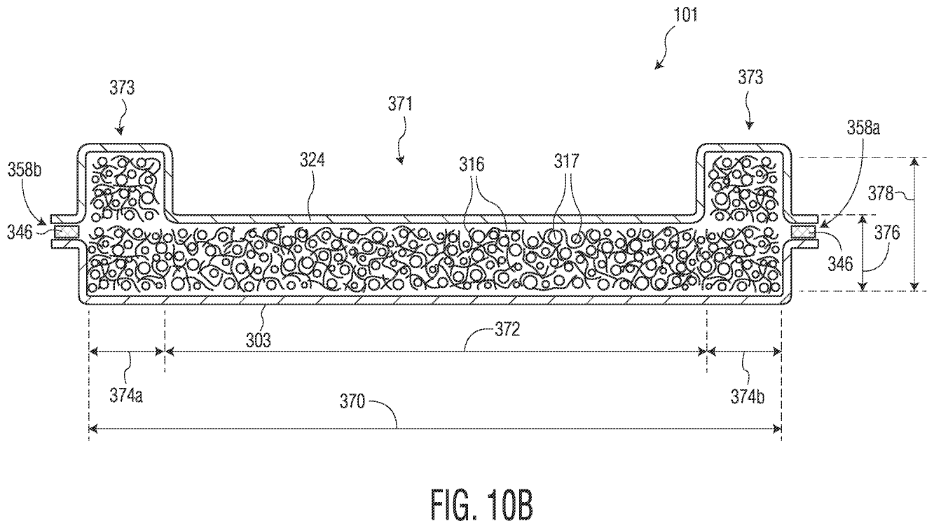

According to some aspects of the present disclosure, the absorbent body 34, or at least one component of the absorbent body 34, may comprise an absorbent structure, such as those structures 101 described in more detail with respect to FIGS. 9A-9C and 10A-10B. In general, such absorbent structures 101 may have one or more layers of absorbent material disposed between a first web material and a second web material. In some examples, the absorbent structure 101 is the entirety of the absorbent body 34. Alternative examples include where the absorbent structure 101 comprises only a portion of the absorbent body 34. In such examples, the absorbent structure 101 may be contained within the absorbent body 34 along with other material, such as additional absorbent materials and/or one or more other web materials. Such other materials, along with absorbent structure 101, which in total form the absorbent body 34, may generally be identified as being part of the absorbent body 34 by their inclusion under fluid transfer layer 46—which may or may not wrap around side edges of the absorbent body 34 in different embodiments—and above the outer cover 26 or spacer layer 48. By contrast, the absorbent body 34 and the fluid transfer layer 46, disposed between the spacer layer 48 or the outer cover 26 and the bodyside liner 28, may together comprise the absorbent system of the articles 10, 210.

In particularly advantageous examples, such as those of FIGS. 9A-9C and 10A-10B, the absorbent material content of the absorbent structure can comprise mostly superabsorbent material, by weight of the absorbent material of the absorbent structure. For example, the absorbent material content of the absorbent structure, by weight of the absorbent material of the absorbent structure, can comprise greater than about 80% superabsorbent material, greater than about 85% superabsorbent material, greater than about 90% superabsorbent material, greater than about 95% superabsorbent material. In such embodiments, the remaining absorbent material content may comprise fibrous absorbent material, such as cellulosic fibers, or any other suitable absorbent material. Additional embodiments of the structures of FIGS. 9A-9C and 10A-10B include where the absorbent material content of the absorbent structure comprises 100% superabsorbent material, by weight of the absorbent material of the absorbent structure, providing for a cellulose-free absorbent structure—and body 34 where the structure 101 is the entirety of the body 34. As will be described in more detail below, particular embodiments of absorbent articles 10, 210 having a high superabsorbent material content, by weight of absorbent material, allow for a good balance between fluid intake rate of the article as well as fit and comfort of the article—as detailed through low article void volumes, allowing such articles 10, 210 to be thinner and more flexible than past absorbent articles.

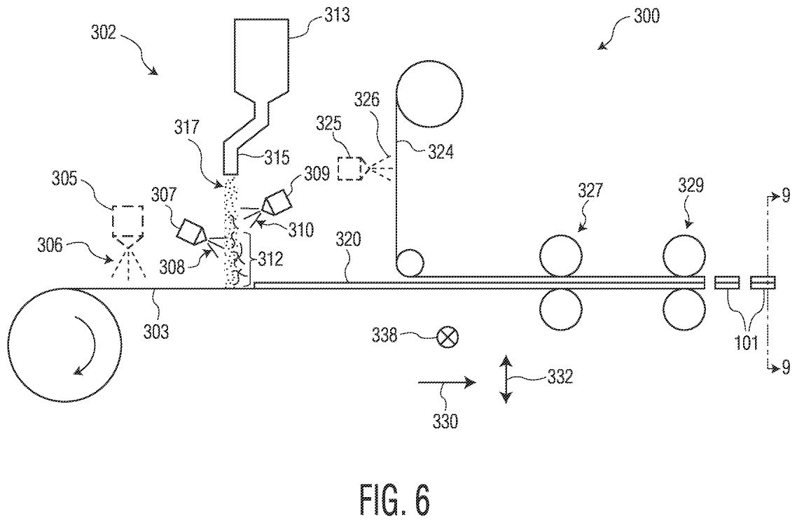

Absorbent structures according to the present disclosure may be formed according to the processes disclosed herein, such as processes 300, 400 detailed in FIGS. 6-8. Such absorbent structures formed by processes 300, 400 may advantageously provide greater thinness, flexibility, superabsorbent material capture, intake and/or rewet performance, and less overall material usage than absorbent structures formed by different processes and/or comprising different material or different relative amounts of material. Although the FIGS. 1-5 focus on description of a diaper absorbent article 10, 210, it should be understood that the absorbent structure of the present disclosure may be used in any absorbent article—including but not limited to diapers, diaper pants, training pants, absorbent inserts, youth pants, swim pants, feminine hygiene products, including, but not limited to, menstrual pads or pants, incontinence products and other adult care garments, medical garments, surgical pads and bandages, other personal care or health care garments, and the like.

FIG. 6 is an exemplary schematic depiction of absorbent structure formation process 300 for forming absorbent structures such as structures 101. Process 300 includes unwinding web material 303 and moving the web material 303 in a machine direction 330. In some particular examples, the web material 303 is a corewrap material of an absorbent structure. The web material 303 moves in the machine direction 330 and arrives at an absorbent material deposition station 302. At the absorbent material deposition station 302, superabsorbent material 317, for example superabsorbent particles, intermixes with one or more adhesives 308, 310 prior to depositing onto the web material 303, for example in a mixing region 312, and ultimately deposits onto the web material 303.

The superabsorbent material 317 flows from hopper 313 and through a chute 315 toward the web material 303. The hopper 313 may be a bulk solid pump or feeder configured to maintain a controlled flow of the superabsorbent material 317 through the absorbent material deposition station 302. The flow rate of the superabsorbent material 317 out of the hopper 313 may be adjustable such that the hopper 313 can deliver different amounts of superabsorbent material 317, resulting in different basis weights of superabsorbent material 317 in the finished absorbent structures 101. Such differences in basis weights of superabsorbent material 317 may allow the formed absorbent structures 101 to be used in different absorbent end uses—such as in diapers, feminine articles, adult care garments, bandages and the like.

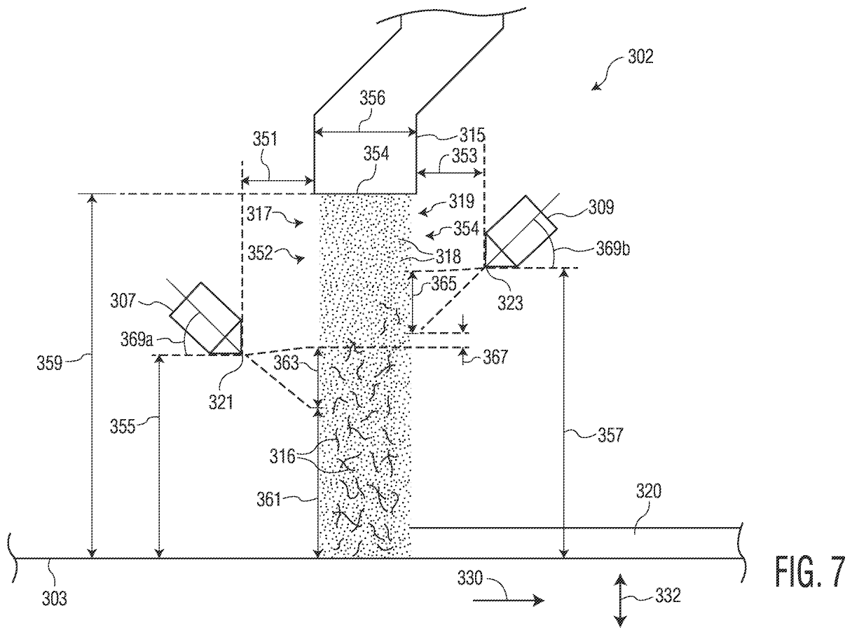

The chute 315 has a chute end 354 (as seen in FIG. 7) that is shown oriented in the vertical direction 332 such that the superabsorbent material 317, shown as individual particles 318 in FIG. 7, exits the chute 315 falling substantially in the vertical direction 332. The superabsorbent material 317 may preferably be fed through the absorbent material deposition station 302 by gravity, without any additional external energy such as a pneumatic force. As used herein, the vertical direction 332 is used to denote a direction perpendicular to the web material 303. The machine direction 330 is a direction parallel within the web material 303 and, accordingly, is perpendicular to the vertical direction 332. In examples where the web material 303 is oriented in a horizontal direction with respect to gravity (e.g. perpendicular to the direction of gravity), the vertical direction 332 may be substantially aligned with respect to gravity. However, in other examples, the vertical direction 332 may be at an angle with respect to gravity—for example angles of up to 25 degrees difference with respect to gravity may be suitable for the vertical direction 332. Accordingly, in such embodiments, the web material 303 may not be moving in a horizontal direction with respect to gravity and the superabsorbent material 317 would fall toward the web material 303 having a direction including a component in both the vertical direction 332 and the machine direction 330 (or potentially opposite the machine direction 330).

Additionally, regardless of the orientation of the vertical direction 332 with respect to gravity, the chute 315 may further be oriented in a non-perpendicular fashion with respect to the web material 303. For instance, the chute end 354 may be oriented perpendicular with respect to the web material 303 (as shown in FIG. 7) or it may be oriented so as to form an angle of greater than 0 degrees and less than about 25 degrees with respect to a direction perpendicular to the web material 303.

In general, the amount of superabsorbent material 317 fed through the absorbent material deposition station 302 can be configured to result in absorbent structures 101 comprising superabsorbent material 317 disposed in amounts between about 50 gsm and about 1000 gsm, or between about 100 gsm and about 1000 gsm, or between about 150 gsm and about 1000 gsm, or between about 200 gsm and about 800 gsm, or between about 250 gsm and about 800 gsm, or between about 300 gsm and about 700 gsm, or between about 350 gsm and about 700 gsm, or between about 400 gsm and about 700 gsm, or between about 450 gsm and about 700 gsm, or between about 500 gsm and about 700 gsm, or between about 400 gsm and about 600 gsm, or between about 500 gsm and about 600 gsm. Such superabsorbent material 317 basis weight values for absorbent structures may be particularly suitable for use in absorbent garments and feminine hygiene products. Although, further absorbent structures that may be formed according to aspects of the present disclosure can have even smaller basis weights of superabsorbent material 317, such as between about 5 gsm and about 50 gsm, or about 5 gsm and about 30 gsm, or between about 10 gsm and about 30 gsm.

The chute opening 354 has an opening width 356 (as measured where the superabsorbent material 317 exits the chute 315) in the machine direction 330. The opening width 356 may be between about 2 mm and about 30 mm, or between about 5 mm and about 25 mm, or between about 5 mm and about 20 mm, or between about 7 mm and about 15 mm. More specifically, opening widths 356 of between about 2 mm and about 10 mm are preferred when the amount of superabsorbent material 317 deposited by the absorbent material deposition station 302 is between about 50 gsm and about 300 gsm. Conversely, opening widths 356 of between about 10 mm and about 14 mm are preferred when the amount of superabsorbent material 317 deposited by the absorbent material deposition station 302 is between about 300 gsm and about 500 gsm, and opening widths 356 of between about 14 mm and about 20 mm are preferred when the amount of superabsorbent material 317 deposited by the absorbent material deposition station 302 is between about 500 gsm and about 1000 gsm.

These combinations of features—the gravity feed method and the chute opening width 356—may help to generate a “sheet” or “stream” of superabsorbent material 317 flowing toward the web material 303. The specified widths 356 may help to ensure that the stream 319 of superabsorbent material 317 has a sufficient width and/or density—particularly at the points where the adhesives 308 and/or 310 contact the stream 319, which can allow for the adhesives 308 and/or 310 to better penetrate the stream 319 and intermix with the superabsorbent material 317. These configurations can help to drive beneficial properties of the resulting absorbent structures 101, as described in more detail below. In some further embodiments, air streams or air curtains may be used to help shape the stream 319 and/or to maintain a desired width and/or density of the stream. In such embodiments, the superabsorbent material 317 might be directed toward the web material 303 to some extent faster than solely by gravity, but such embodiments would still be considered to comprise a gravity feed system because the superabsorbent material 317 is not pneumatically or otherwise forced from the chute end 354.

As the superabsorbent material 317 falls toward the web material 303, adhesive applicators 307 and/or 309 spray adhesive 308 and/or 310 toward the falling superabsorbent material 317. The adhesive 308 and/or 310 intermixes with the falling superabsorbent material 317 prior to the mixture of the superabsorbent material 317 and the adhesive 308 and/or 310 depositing onto the web material 303. FIG. 7 is a close-up schematic depiction of the absorbent material deposition station 302, showing more details regarding the adhesive applicators 307 and/or 309, the adhesives 308 and/or 310.

The adhesives 308 and/or 310 applied by adhesive applicators 307 and/or 309 may generally be applied at add-on percentages of less than about 7%, or less than about 6%, or less than about 5%, or less than about 4%, or less than about 3%, or less than about 2%. In other embodiments, the add-on percentages may be between about 1% and about 7%, or between about 1.5% and about 7%, or between about 2% and about 6%, or between about 2% and about 5.5%, or between about 2% and about 5%, or between about 2% and about 4.5%, or between about 2% and about 4%, or between about 2.5% and about 4%, or between about 3% and about 4%. As used herein, the term “add-on” amount or percentage is the amount of adhesive added such that a resulting weight of the adhesive within the absorbent structure has the desired relation to a weight of the absorbent material within such absorbent structure. As one illustrative example, where the superabsorbent material 317 is disposed in an absorbent structure at a basis weight of 500 gsm, and where the adhesives 308 and/or 310 were applied at a combined add-on rate of 5%, the resulting basis weight of the adhesive 308 and/or 310 in the formed absorbent structure would be 25 gsm (5% of the 500 gsm basis weight of the superabsorbent material 317).

As shown in FIGS. 6-8, the absorbent material deposition station 302 can comprise two adhesive applicators 307 and 309. The first adhesive applicator 307 may be positioned upstream (relative to the process direction 330) of the chute 315 while the second adhesive applicator 309 may be positioned downstream of the chute 315. The superabsorbent material 317 may form a stream 319 of superabsorbent material 317 as it falls toward the web material 303. Where the adhesive applicator 307 is positioned on the upstream side of the chute 315, the adhesive applicator 307 is configured to spray the first adhesive 308 at the first side 352 of the stream 319 of superabsorbent material 317.

The adhesive applicator 307 may be configured to spray the first adhesive 308 such that the first adhesive 308 contacts the first side 352 of the stream 319 of superabsorbent material 317 along a portion of the stream 319 having a length 363 along the stream 319. In some embodiments, the length 363 may be insubstantial in that the first adhesive 308 may be sprayed as a stream which has minimal-to-no spread. However, in other embodiments, the first adhesive 308 may have some spread and accordingly the length 363 could be between about 2 mm and about 10 mm, or between about 2 mm and about 6 mm, or between about 2 mm and about 4 mm.

In order to allow sufficient time for the first adhesive 308 to intermix with the stream 319 of the superabsorbent material 317 prior to the mixture of the first adhesive 308 and the superabsorbent material 317 depositing onto the web material 303, the first adhesive 308 desirably contacts the stream 319 at a first contact point located a distance 361 away from the web material 303. The distance 361 may be between about 4 mm and about 40 mm, or between about 4 mm and about 35 mm, or between about 5 mm and about 30 mm, or between about 6 mm and about 25 mm. Where the first adhesive 308 is sprayed in a spread fashion and contacts the stream 319 along the length 363, the first contact point, and accordingly the distance 361, is measured with respect to the center of the length 363 along which the first adhesive 308 contacts the stream 319.

To achieve such distances 361, the nozzle 321 is positioned a distance 355 away from the web material 303 and a distance 351 away from the chute 315. These distances 355, 351 may be adjusted to achieve the desired distance 361. As some non-limiting examples, the distance 355 may generally be between about 5 mm and about 40 mm, or between about 10 mm and about 30 mm. As a comparison, the chute 315 may be positioned a distance 359 away from the web material 303. The distance 359 may be between about 50 mm and about 90 mm, or between about 60 mm and about 80, or between about 70 mm and about 80 mm. Distances 359 higher than about 70 mm, or about 80 mm, or about 90 mm may result in undesirable spreading of the stream 319. Distances lower than about 60 mm or about 50 mm may result in there being insufficient space between the chute 315 and the web material 303 to allow for sufficient mixing of the superabsorbent material 317 and the first adhesive 308 (or the second adhesive 310 described in more detail below).

It has been further found that the angle 369a at which the nozzle 321 is oriented with respect to the machine direction 330 may be important in achieving a desired level of mixing between the first adhesive 308 and the stream 319. Preferably, the angle 369a is between about 40 degrees and about 80 degrees, or between about 45 degrees and about 75 degrees, or between about 50 degrees and about 70 degrees.

The adhesive applicator 309 may be configured similarly to the adhesive applicator 307. The adhesive applicator 309 can spray the second adhesive 310 such that the second adhesive 310 contacts the second side 354 of the stream 319 of superabsorbent material 317 along a portion of the stream 319 having a length 365 along the stream 319. Accordingly, the length 365 may be insubstantial in that the second adhesive 310 may be sprayed as a stream which has minimal-to-no spread. In other configurations, the second adhesive 310 may have some spread such that the length 365 may vary between about 2 mm and about 10 mm, or between about 2 mm and about 6 mm, or between about 2 mm and about 4 mm.

To allow sufficient time for the second adhesive 310 to intermix with the stream 319 of the superabsorbent material 317 prior to the mixture of the second adhesive 310 and the superabsorbent material 317 depositing onto the web material 303, the second adhesive 310 preferably contacts the stream 319 at a second contact point on the stream 319 located a distance away from the web material 303 equal to distance 367 added to distance 361. The distance 367 plus distance 361 may generally be between about 4 mm and about 40 mm, or between about 4 mm and about 35 mm, or between about 5 mm and about 30 mm, or between about 6 mm and about 25 mm. Additionally, where the second adhesive 310 is sprayed in a spread fashion and contacts the stream 319 along some length 365, the second contact point, as well as the distance 367 added to the distance 361, is measured with respect to the center of the length 365 along which the second adhesive 310 contacts the stream 319 (and with respect to the center of the length 363 if the first adhesive 308 contact the stream 319 for some appreciable length 363).

It can be understood that the distance 361 and the distance 367 added to the distance 361 overlap in their preferred ranges. According to some preferred embodiments, the distance 361 is less than the distance 367 added to the distance 361. For example, it may be preferred that the applicator 307 is positioned closer to the web material 303 than the applicator 309. In such embodiments, the distance 361 may be preferred to be between about 4 mm and about 22 mm, or between about 4 mm and about 20 mm, or between about 6 mm and about 15 mm. The distance 367 added to the distance 361 may be greater than the distance 361 by between about 5 mm and about 15 mm, or between about 6 mm and about 13 mm, or between about 6 mm and about 11 mm, e.g. the distance 367 may be between about 5 mm and about 15 mm, or between about 6 mm and about 13 mm, or between about 6 mm and about 11 mm. In such embodiments, the distance 367 may represent a spacing between the first contact point where the first adhesive 308 contacts the stream 319 and the second contact point where the second adhesive 310 contacts the stream 319.

It has been found that spraying the adhesives 308 and/or 310 at the stream 319 may cause the stream 319 to bend in the direction of the spray. Without being limited by theory, it is thought that the force of the adhesives 308 and/or 310 contacting the stream and/or the optional pattern air supplied by the applicators 307 and/or 309 can cause this bending of the stream 319. Accordingly, where the first contact point where the first adhesive 308 contacts the stream 319 is at a lower point than the second contact point where the second adhesive 310 contacts the stream 319, the stream 319 may bend in the machine 330 just prior to depositing onto the web material 303. This bending of the stream 309 in the machine direction 330 helps to ensure a smooth deposition of mixture of the superabsorbent material 317 and the first adhesive 308 (and optionally the second adhesive 310), resulting in a more uniform mixture 320, which has many benefits in terms of capture and stabilization of the superabsorbent material 317, integrity of the resulting absorbent structure 101, and uniformity of the distribution of the superabsorbent material 317 and the first adhesive 308 (and optionally the second adhesive 310).

As with the nozzle 321, the nozzle 323 may be positioned a distance 357 away from the web material 303 and a distance 353 away from the chute 315 in order to achieve the desired distance 367 added to the distance 361. The angle 369b at which the nozzle 323 is oriented with respect to the web of material 303 may further be similar to the angle 369a. For example, the angle 369b may vary between about 40 degrees and about 80 degrees, or between about 45 degrees and about 75 degrees, or between about 50 degrees and about 70 degrees. In at least some embodiments, the angle 369a and the angle 369b can be the same, while in other embodiments the angles 369a, 369b are different.

The applicators 307 and/or 309 may be preferably configured to spray the adhesives 308 and/or 310 in a substantially random pattern. It has been found that spray patterns that are more randomized, irregular, or erratic may produce better results in terms of performance of the absorbent structures 101—such as in terms of capture and stabilization of the superabsorbent material 317, integrity of the resulting absorbent structure 101, and uniformity of the distribution of the superabsorbent material 317 and the adhesives 308 and/or 310. One such exemplary spray pattern is the pattern produced by the Universal™ Signature™ Spray Nozzles available from the Nordson Corporation having headquarters at 28601 Clemens Road, Westlake, OH 44145 USA. However, in other embodiments, different adhesive spray patterns which are more regular and less randomized, but still considered a random pattern, may be sufficient to produce absorbent structures 101 having desirable performance properties. It is further contemplated that some non-random spray patterns may also be sufficient to produce absorbent structures 101 having desirable performance properties.

Although shown in FIG. 7 as comprising two adhesive applicators 307 and/or 309, in some embodiments the absorbent material deposition station 302 may only comprise one of the adhesive applicators 307 and/or 309. Further, although shown, and described above, with the adhesive applicator 307 directing adhesive 308 to the first side 352 of the stream 319 (which is the upstream side of the stream 319) positioned closer to the web material 303 than the adhesive applicator 309, this orientation is not required in all embodiments. For example, in further embodiments, the adhesive applicator 307 may be positioned further away from the web material 303 than the adhesive applicator 309, while still being positioned on the upstream side of the stream 319. In any of these such embodiments, the distances between the first contact point and the second contact point with respect to each other and with respect to the web material described previously may be reversed. That is, the distance 361 may describe the distance between the second contact point and the web material 303 while the distance 367 added to the distance 361 may describe the distance between the first contact point and the web material 303 (with the distance 367 describing the distance between the first contact point and the second contact point).

As the web 303 passes the absorbent material deposition station 302, a deposited mixture 320 of the mixture of the adhesives 308 and/or 310 and the superabsorbent material 317 forms as a matrix of adhesive and superabsorbent material 317. Generally, the adhesives 308 and/or 310, in the mixing region 312, form a mesh network with the superabsorbent material 317 captured therein. When this mesh network is deposited onto the web 303, the adhesives 308 and/or 310 forms a three-dimensional mesh network with the superabsorbent material 317 immobilized therein.

During the deposition of the mixture 320, vacuum energy may optionally be applied to the web material 303. For example, web material 303 may be supported by a forming surface—such as a forming belt or forming drum as is typical in the art. Vacuum energy may be applied to the forming surface such that air is drawn through the forming surface from the side where the web material 303 is located. Accordingly, the web material 303, along with the mixture 320 as it is falling toward the web material 303, is drawn to the forming surface due to the applied vacuum energy. Such vacuum energy may help to control the spread of the mixture 320 as it is falling toward the web material 303, thereby helping to form a relatively more uniform absorbent structure 101. It has been found that particularly high-pressure differentials are preferred at the forming surface—above and beyond typical pressure differentials in the art. For example, it may be preferred that the vacuum energy produces a pressure differential of greater than about 0.25 m of water at the forming surface. In further embodiments, it may be more preferable for even higher-pressure differentials, such as greater than about 0.35 m of water, or greater than about 0.5 m of water, or greater than about 0.65 m of water, as measured at the forming surface.

A web material 324 may further be applied to the deposited mixture 320. In some embodiments, an adhesive applicator 325 may spray an adhesive 326 onto the web material 324 prior to the web material 324 being positioned onto the deposited mixture 320. Although, it should be understood that the adhesive applicator 325 is only optional and may not be present in some embodiments. Where present, the applied adhesive 326 may operate to more closely couple the web material 324 to the deposited mixture 320 and/or to further immobilize the superabsorbent material 317 within the formed absorbent structure.

According to some aspects of the present disclosure, the combination of the web material 303, the deposited mixture 320, the reinforcing web material 324, and the web material 324 may pass through one or more nip stations 327 to help compress the components together. In general, the nip station 327 may apply a pressure to the combination of the web material 303, the deposited mixture 320, the reinforcing web material 329, and the web material 324 of between about 0.5 pounds per linear inch (PLI) (88 N/m) and about 1.5 PLI (263 N/m), or between about 0.75 PLI (131 N/m) and about 1.25 PLI (219 N/m). Such pressures help to further connect the deposited mixture to the web materials 303, 324. Although not required in all embodiments, it may be preferred for the nip station 327 to be positioned in relatively close proximity to the material deposition station 302 such that the adhesives 308 and/or 310 are still open and unset when the combination of the web material 303, the deposited mixture 320, and the web material 324 passes through the nip station 327.

After the one or more nip stations 327, the combination of the web material 303, the deposited mixture 320, and the web material 324 may pass to a cutting station 329 where the connected length of the web material 303, the deposited mixture 320, and the web material 324 is cut into individual absorbent structures 101. These individual absorbent structures 101 may then be combined into a manufacturing process for producing the various absorbent products described herein.

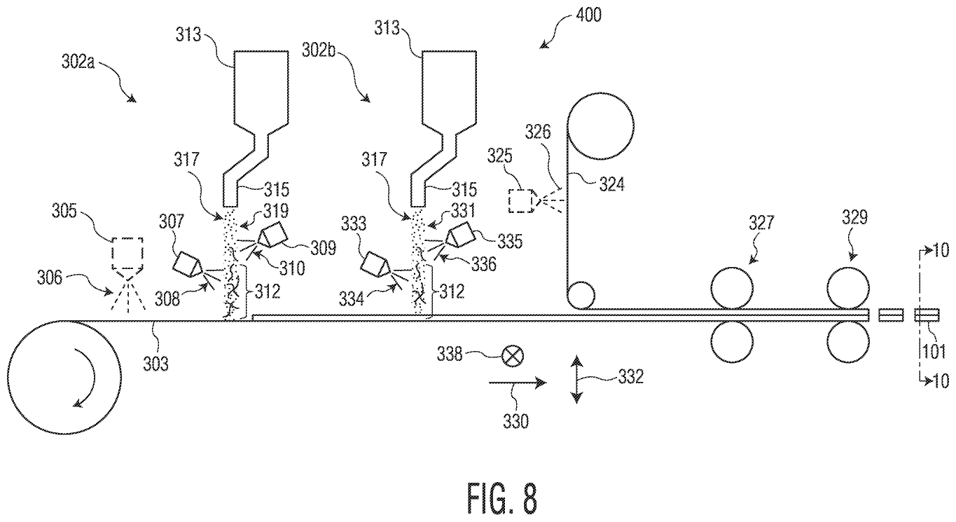

FIG. 8 depicts is an exemplary schematic depiction of an alternative absorbent structure formation process 400. The process 400 is similar to process 300, except the process 400 employs two absorbent material deposition stations 302a, 302b. It has been found that there are some advantages of employing two absorbent material deposition stations 302a, 302b over a single absorbent material deposition station 302, thus forming two separate absorbent layers. For example, as the desired amount of deposited superabsorbent material 317 gets higher, the lower the ability of a single absorbent material deposition station 302 to form an absorbent structure 101 having desired performance properties. For example, if the desired amount of deposited superabsorbent material 317 gets too high, a single absorbent material deposition station 302 may not be able to form a mixture of superabsorbent material 317 and adhesives 308 and/or 310 which sufficiently immobilizes the superabsorbent material 317—particularly at desired low adhesive add-on amounts. For instance, in such examples, a superabsorbent material capture property of such formed absorbent structures 101 may fall below a desired value. In still further contemplated embodiments of the present disclosure, a method of forming absorbent structures 101 may include more than two absorbent material deposition stations—for example three, four, or five absorbent material deposition stations.

Conversely, the same desired amount of deposited superabsorbent material 317 may be able to be sufficiently immobilized by employing two absorbent material deposition stations 302a, 302b such that the resulting absorbent structures 101 have a desired superabsorbent material capture value. Additionally, employing two absorbent material deposition stations 302a, 302b may allow for increased production rates—even at lower superabsorbent material 317 amounts and higher adhesive add-on amounts. Accordingly, in the process 400, after the mixture of superabsorbent material 317 and adhesives 308 and/or 310 is deposited onto the web material 303 at absorbent material deposition station 302a (which may be equivalent to absorbent material deposition station 302 of FIGS. 6 and 7), the web material 303 and the deposited mixture of superabsorbent material 317 and adhesives 308 and/or 310 moves onto absorbent material deposition station 302b.

Similar to absorbent material deposition station 302a, absorbent material deposition station 302b may be configured to direct a second stream 331 of superabsorbent material 317 toward the web 303 and the already deposited mixture of superabsorbent material 317 and adhesives 308 and/or 310. The absorbent material deposition station 302b may comprise adhesive applicators 333 and/or 335 which may spray adhesive 334 and/or 336 toward the second stream 331 of falling superabsorbent material 317. The adhesive 334 and/or 336 intermixes with the falling superabsorbent material 317 prior to the mixture of the superabsorbent material 317 of the second stream 331 and the adhesive 334 and/or 336 depositing onto the web material 303 and the previously deposited mixture of superabsorbent material 317 and adhesives 308 and/or 310.

According to some aspects of the present disclosure, the absorbent material deposition station 302b can comprise two adhesive applicators 333 and 335. With respect to the absorbent material deposition station 302b, the first adhesive applicator 333 (which may be the third adhesive applicator of the process 400) may be positioned upstream (relative to the process direction 330) of the chute 315 of the second deposition station 302b while the second adhesive applicator 335 (which may be the fourth adhesive applicator of the process 400) may be positioned downstream of the chute 315 of the second deposition station 302b. The adhesive applicator 333 is configured to spray the first adhesive 334 (which may be the third adhesive of the process 400) at a first side of the second stream 331 of superabsorbent material 317. The adhesive applicator 335 is configured to spray the second adhesive 336 (which may be the fourth adhesive of the process 400) at a second side of the second stream 331 of superabsorbent material 317.

Generally, the positions, locations, distances, and other features and optional components or features of the absorbent material deposition station 302 described with respect to FIG. 7 may be the same as for absorbent material deposition station 302a. Likewise, the absorbent material deposition station 302b may be the same or substantially similar to the absorbent material deposition station 302a. The absorbent material deposition station 302b may be positioned between about 0.25 m and about 3.0 m, or more preferably between about 0.25 m and about 2.0 m, or even more preferably between about 0.25 m and about 1.0 m away from the absorbent material deposition station 302a.

Turning back to web materials 303 and 324, as shown in FIGS. 6 and 8, the web material 324 may be coupled to the deposited mixture 320 of the superabsorbent material 317 and the adhesives 308, 310, 334, and/or 336 to form the absorbent structures 101. Some alternative embodiments according to aspects of the present disclosure may forgo the web material 324 altogether. In such embodiments, the web material 303 may be wide enough that, after the mixture 320 is deposited onto the web material 303, the web material 303 is wrapped around the mixture 320 to form the absorbent structure 101.

FIGS. 9A-9C depict different cross-sections of exemplary absorbent structures 101 according to aspects of the present disclosure. The cross-sections representing FIGS. 9A-9C are taken along line 9-9 of FIG. 6, showing different configurations of the mixture 320, the web material 303, and web material 324, where present.

FIG. 9A depicts an embodiment of an absorbent structure 101 of the present disclosure comprising web material 303 and web material 324, with mixture 320 disposed between web material 303 and web material 324. The web material 303 and web material 324 may have top surfaces 342 and 344, respectively, and bottom surfaces 343 and 345, respectively. In some exemplary embodiments according to FIG. 9A, the mixture 320 may be disposed on top surface 342 of web material 303 and on bottom surface 345 of web material 324. In some of these embodiments, absorbent structure 101 may further comprise seaming adhesives 346 disposed outboard of the mixture 320 and bonding the bottom surface 345 of web material 324 to the top surface 342 of web material 303. Such seaming adhesives 346 may help to seal the side edges 358a, 358b of the absorbent structure 101 closed. However, it should be understood that such adhesives 346 are not necessary in all embodiments—many embodiments sufficiently capture the superabsorbent material 317 such that little to no superabsorbent material 317 may escape out of the absorbent structure 101, even without the seaming adhesive 346.

Where present, the seaming adhesives 346 may be applied by adhesive applicators prior to or after deposition of the mixture 320 (for example, optional adhesive applicators 305 and/or 325 may apply the seaming adhesives 346). Alternatively, the seaming adhesives 346 may be applied during deposition of the mixture 320 by adhesive applicators 307, 309, 333 and/or 335, for example where the adhesive spray from the adhesive applicators 307, 309, 333 and/or 335 is wider than the stream or streams of superabsorbent material 317. In other embodiments, however, absorbent structure 101 may not include any seaming adhesives 346. In such embodiments the adhesives 308, 310, 334, and/or 336 may be sufficient to bond the web material 303 to the web material 324.

FIG. 9B depicts another embodiment of an absorbent structure 101 of the present disclosure comprising web material 303 and web material 324, with mixture 320 disposed between web material 303 and web material 324. In this embodiment, in contrast to the embodiment of FIG. 9A, instead of the bottom surface 345 of web material 324 bonded to the top surface 342 of web material 303, the top surface 344 of web material 324 may be bonded to the top surface 342 of web material 303. For example, the web material 324 may wrap at least partially around the mixture 320, sometimes termed a C-wrap, such that the bottom surface 345 of web material 324 is disposed about both a portion of a first side of the mixture 320 and a second side of the mixture 320. In the embodiment shown in FIG. 9B, the web material 324 may be disposed between the mixture 320 and the web material 303 where the web material 324 and the web material 303 overlap. Although, in other embodiments, the web material 324 may wrap around both of the mixture 320 and the web material 303 such that the web material 303 is disposed between the mixture 320 and the web material 324 where the web material 324 and the web material 303 overlap.

In the embodiment shown in FIG. 9B, the absorbent structure 101 may include seaming adhesives 346 connecting the top surface 344 of web material 324 to the top surface 342 of web material 303 proximate lateral edges of the absorbent structure 101. Although, it should be understood that such seaming adhesives 346 are optional and may not be present in all embodiments. Where present, seaming adhesives 346 may be applied for example, by optional adhesive applicators 305 and/or 325, or may be applied by one or more of adhesive applicators 307, 309, 333 and/or 335.

FIG. 9C depicts another embodiment of an absorbent structure 101 of the present disclosure comprising only web material 303. In this embodiment, web material 303 wraps around the mixture 320, for example forming a C-wrap configuration. As shown in FIG. 9C, the web material 303 has web end portions 347 and 349. In some exemplary embodiments according to FIG. 9C, the web material 303 may wrap around the mixture 320 such that the web end portions 347 and 349 overlap each other. As shown in FIG. 9C, such configurations may further include one or more seaming adhesives 346 disposed between the web end portions 347 and 349 and bonding the web end portions 347 and 349 of the material 303 together. Although, such seaming adhesives 346 are optional and may not be present in other embodiments. In further embodiments according to FIG. 9C, the web end portions 347 and 349 may be spaced from each other such that the web end portions 347 and 349 do not overlap. In such embodiments, a portion of the mixture 320 may be left uncovered by the web material 303.

With respect to FIGS. 9A-9C, the exemplary absorbent structures 101 may have top sides 362 and bottom sides 364. However, it should be understood that these absorbent structures 101 may be used in any orientation. For example, in some instances, the described absorbent structures 101 may be placed into an absorbent article, such as article 10, with the top side 362 disposed most closely to the body facing surface 19. In other instances, the absorbent structures 101 may be placed into an absorbent article such as article 10 with the bottom side 364 disposed most closely to the body facing surface 19.

Where web material 303 forms the top side 362 of the absorbent structure 101 and where the top side 362 is disposed most closely to the body facing surface 19, the web material 303 may be any suitable nonwoven material—for instance, a bonded carded web, a meltblown material, a spunbond material, including spunbond and meltblown combination webs commonly referred to SMS webs or SMMS webs or the like, a spunlace material, a hydroentangled material, an airlaid material, a coform material, or may be a material formed according to mixtures of technologies used to form the above described materials such as a spunbond-meltblown-spunbond material or other such similar materials. Typical basis weights for such web materials 303 may range between about 8 gsm to about 200 gsm, or between about 10 gsm and about 150 gsm, or between about 10 gsm and about 100 gsm, or between about 10 gsm and about 80 gsm. Alternatively, the web material 303 may be formed of wetlaid fibrous materials such as uncreped through air dried tissue or creped tissue, or other material sheets made from cellulosic fibers. The web material 303 may further comprise a combination of nonwoven and fibrous materials, including fiberized pulp captured on top of or between nonwoven materials or wetlaid fibrous materials. In such embodiments, the fiberized pulp may be densified to form the web material 303—prior to its use in capturing superabsorbent 317 and adhesive 308 and/or 310.

Without respect to any specific type of material, it has been found that web material 303 should ideally have sufficient air permeability to allow vacuum air flow to pass through the web material 303 and to at least partially entrain the streams 319 (and optionally 331) of superabsorbent material 317 and adhesives 308 and/or 310 (and optionally 334 and/or 336) in such vacuum air flow. For example, it has been found that an air permeability of the web material 303 should be greater than about 50 standard cubic feet per minute (SCFM) of air (0.71 standard cubic meters per minute (SCMM)). In further embodiments, it may be more preferable for the web material 303 to have an air permeability of greater than about 100 SCFM (1.4 SCMM), or greater than about 200 SCFM (2.1 SCMM). Such measurements of air permeability may be made consistent with standard industry practices for measuring air permeability. More specifically, the measurements may be made according to the ASTM D737 and using a 38 cm² sample at a pressure of 125 Pa. These air permeability measurements may be made with a Frazier Instruments Model LP air permeability tester from Frazier Instruments (offices in Hagerstown, Maryland), a Textest FX 3300 air permeability unit from Textest (offices in Schwerzenbach, Switzerland), or equivalent test unit.

Again, where the web material 303 forms the top side 362 of the absorbent structure 101 and where the top side 362 is disposed most closely to the body facing surface 19 it may be preferable that the fibers, or at least the surface fibers, of the web material 303 have sufficient wettability to allow fluid intake, fluid flow, and fluid distribution through the web material 303 to the superabsorbent material 317. In some embodiments, the wettability may come from the composition of the fiber. For instance, the fibers forming the web material 303 may be inherently wettable fibers include such as natural cellulosic fibers derived from cotton, wood, or other fibers. Other examples of an inherently wettable fiber include a reconstituted cellulosic fiber such as a rayon fiber. In further embodiments, the fibers forming the web material 303 may not be inherently wettable, but may be changed to be wettable, such as by addition of a surfactant treatment to the fibers, or at least to the surface fibers. The surfactant treatment may be applied at least to the surface fibers in a continuous or discontinuous manner. In other embodiments, a surfactant treatment can be added internally to the fiber which will ultimately migrate to the surface of the fiber.

Where web material 324 forms the bottom side 364 of the absorbent structure 101 and where the bottom side 364 is disposed most closely to the body facing surface 19, the web material 324 may be any suitable nonwoven material—for example, any of those recited with respect to web material 303. Additionally, the web material 324 may be preferred to have any of the same properties as were described above with respect to web material 303. Where web material 324 forms the bottom side 364 of the absorbent structure 101 and where the top side 362 is disposed most closely to the body facing surface 19, the web material 324 may also be any of the materials described above with respect to web material 303, including having any of the properties and their described ranges.

The adhesives 308 and/or 310 may generally comprise hot-melt adhesives, and the nozzles 321, 323 may be configured to project the adhesives 308 and/or 310 toward the stream 319 of superabsorbent material 317 such that the adhesives 308 and/or 310 form adhesive filaments 316. Desirably, the adhesives 308 and/or 310 should have sufficient tack and cohesion. An exemplary suitable adhesive is the TECHNOMELT DM 5402U adhesive available from Henkel Corporation, a company having offices in Rocky Hill, Connecticut. This suitable adhesive is a styrenic block copolymer based hot melt adhesive design to have high cohesion and strong specific adhesion to provide good fixation of the superabsorbent material 317 in the absorbent structure under both wet and dry conditions. It may further be generally preferable for the adhesives 308 and/or 310 to be non-water soluble in order to help retain the positioning of the superabsorbent material 317 within the structure 101 after one or more liquid insults. It has been found that rubber-based adhesives may be preferable in that they may produce structures 101 which perform superior to other adhesives, such as standard construction adhesives or olefin-based adhesives.

The adhesives 308 and/or 310 may also have suitably low storage modulus (G′). The storage modulus of the adhesives 308 and/or 310 generally refers to the ability of the adhesive composition (after it has set up or otherwise generally dried, e.g., after cooling) to deform, such as upon flexing of the outer cover 26 or other substrate on which the absorbent body 34 is formed, without a substantial loss of integrity of the adhesive composition. By using adhesives 308 and/or 310 having a relatively low storage modulus, the absorbent body 34 is suitably generally soft and flexible to permit flexing of the absorbent body 34 along with the outer cover 26. More specifically, the storage modulus is a coefficient of elasticity representing the ratio of stress to strain as the adhesive composition is deformed under a dynamic load.

As used herein, the storage modulus of the adhesives 308 and/or 310 is reported as measured according to the Rheology Test set forth in U.S. Pat. No. 8,852,381 to Nhan et al. As an example, the storage modulus (G′) measured at 25 degrees Celsius of the adhesives 308 and/or 310 as determined by the Rheology Test is suitably between about 1.0×10\hat{ }3 and about 1.0×10\hat{ }Pa, or more suitably between about 1.0×10\hat{ }3 and about 1.0×10\hat{ }Pa, or more suitably between about 2.5×10\hat{ }4 and about 22.5×10\hat{ }4 Pa.

In general, the adhesive applicators 307 and/or 309 operate to spray the adhesives 308 and/or 310 such that the adhesives 308 and/or 310 form adhesive filaments 316 which contact the stream 319. The adhesive applicators 307 and/or 309 may be generally configured to spray the adhesives 308 and/or 310 such that the adhesives 308 and/or 310 form filaments 316 having preferred diameters. It has been found that it may be preferable for the filaments 316 to have diameters of between 25 micrometers (microns) and 150 microns, or between 50 microns and 100 microns, or between 75 microns and 100 microns. These ranges of filament diameters have been shown to work together well with superabsorbent material 317 having the below described particle diameters in order to provide for beneficial performance properties of the structures 101.

Although the above described adhesive properties have been described with respect to adhesives 308 and/or 310, where present the adhesives 334 and/or 336 may have properties similar to those described above with respect to adhesives 308 and/or 310. Likewise, where present, the applicators 333 and/or 335 may be configured to spray the adhesives 334 and/or 336 in a similar manner to how the adhesive applicators 307 and/or 309 are configured to spray the adhesives 308 and/or 310. For example, the diameters of the filaments 316 formed by the adhesives 334 and/or 336 being sprayed from applicators 333 and/or 335 may be similar to the diameters described above with respect to filaments 316 formed by the adhesives 308 and/or 310 being sprayed from applicators 307 and/or 309.

In the embodiments of absorbent structures 101 of FIGS. 9A-9C, it may be particularly preferred for the mixture 320 to comprise superabsorbent material 317 in amounts between about 100 gsm and about 700 gsm, or more particularly between about 150 gsm and about 700 gsm, or between about 200 gsm and about 700 gsm, or between about 250 gsm and about 700 gsm, or between about 250 gsm and about 600 gsm, or between about 250 gsm and about 550 gsm. In these embodiments, the adhesives 308 and/or 310 may combine to equal an add-on percentage of between about 2% and about 7%, or more particularly between about 2% and about 6%, or between about 2% and about 5.5%, or between about 2% and about 5%, or between about 2% and about 4.5%, or between about 2.5% and about 4.5%, or between about 2.5% and about 4%. It may be particularly advantageous for the adhesives 308 and/or 310 to combine to equal an add-on percentage of less than about 5%, less than about 4.5%, or less than about 4%, or less than about 3.5%, or less than about 3%, or less than about 2.5%. The add-on percentage, in this context, is the amount of adhesive relative to the amount of superabsorbent material 317. Accordingly, as one example, an absorbent structure 101 having an absorbent material 317 basis weight of 500 gsm and an adhesive add-on percentage of 5% would result in the adhesive being present in an amount of about 25 gsm (5% of 500 gsm).

Additionally, it has been found that the sizes of individual particles 318 of the superabsorbent material 317 may drive certain desired properties of the formed absorbent structures 101. For instance, particle size of the individual particles 318 may at least partially drive pad integrity and superabsorbent material capture values, particularly in conjunction with the described structural features of the adhesive filaments 316. For example, it has been found that where the bulk superabsorbent material 317 has mean particle sizes ranging between about 150 and about 1000 micrometers (microns) in diameter provide good results—especially in conjunction with the above-described adhesive filament 316 diameters. In such embodiments, it may be preferred that at least 50% of the mass of the bulk superabsorbent material 317 have diameters larger than about 180 microns. In other embodiments, it may be preferred that at least 60%, or at least 70%, or at least 80% of the mass of the bulk superabsorbent material 317 have diameters larger than about 180 microns. In further embodiments, it may more preferable that at least 50% of the mass of the bulk superabsorbent material 317 have diameters larger than about 300 microns, or that at least 60%, or at least 70%, or at least 80% of the mass of the bulk superabsorbent material 317 have diameters larger than about 300 microns.

Where the bulk superabsorbent material 317 mean particle size is too low, such as lower than about 300 microns, or lower than about 180 microns, the formation and performance of the structures 101 can be affected to a detrimental degree. For example, such small mean particle sizes may affect the ability of the superabsorbent material 317 to fall in a relatively uniform stream from the chute 315, thereby resulting in relatively more non-uniformity of the superabsorbent material 317 and adhesives 308 and/or 310 (and optionally 334 and/or 336). Additionally, such small average particle sizes may begin to approach the average diameter size of the adhesive filaments 316, both affecting capture of the individual particles 318 by the adhesive filaments 316 and reducing absorptive performance because the adhesive filaments 316 would more readily block liquid from accessing all portions of the individual particles 318. Determining the masses of different portions of particles of the bulk superabsorbent material 317 may be performed by any classification process known in the art. For example, it is well known to utilize multiple sieves with differing mesh sizes to separate out different portions of particles from the bulk superabsorbent material 317 having differing particle size diameters. One particular method which can be used in such a classification effort may be ASTM D1921-18, titled “Standard Test Methods for Particle Size (Sieve Analysis) of Plastic Materials”.

According to further aspects of the present disclosure, another way in which the deposition of the absorbent mixture 320 may differ between absorbent material deposition stations 302a, 302b is that the width of the streams 319, 331 of the superabsorbent material 317, in a direction perpendicular to the machine direction 330, termed the cross-machine direction 338 herein, may be different. For example, one of the streams 319, 331 may be narrower in the cross-machine direction 338 than the other of the streams 319, 331 such that the produced absorbent structures 101 have regions of zoned basis weights of superabsorbent material 317 (and adhesives 308, 310, 334, and/or 336). FIGS. 10A-10B depict different exemplary cross-sections of absorbent structures 101 as taken along line 10-10 in FIG. 8 showing such a zoned mixture 320. Accordingly, the absorbent structures 101 of FIGS. 10A-10B represent different exemplary absorbent structures 101 produced by process 400 where the cross-machine direction widths of the streams 319, 331 of superabsorbent material 317 were different, thus resulting in a varying width of the deposited mixture 320 throughout the structures 101. It should be understood that all of these below described embodiments regarding depositing the mixture 320 at different cross-machine direction widths may be further combined with any of the previously described embodiments where the amount of superabsorbent material 317 and/or the amounts of adhesives 308, 310, 334, and/or 336 differ between each of the absorbent material deposition stations 302a, 302b.

FIG. 10A depicts an exemplary cross-section of an absorbent structure 101 having an overall width 370, central zone 371 having a central zone width 372, and side zones 373 having side zone widths 374a, 374b. The central zone width 372 may generally be between about 20% and about 80% of the overall width 370. In more specific embodiments, the central zone width 372 may be between 25% and 75%, or between about 30% and about 70%, or between about 35% and about 65%, or between about 40% and about 60% of the overall width 370. Accordingly, the side zone widths 374a, 374b, added together, may generally be between about 80% and about 20% of the overall width 370, equaling the required percentage of the overall width 370 that, when added to the central zone width 372, equals 100% of the overall width 370. In some embodiments, the side zone widths 374a, 374b may be equal to each other. Although in other embodiments, the side zone widths 374a, 374b may differ from each other by between greater than 0% and less than about 50% of the side zone width 374a, 374b having the greater value. As one illustrative example, the overall width 370 may be 100 mm, the central zone width 372 may be 60 mm, and the side zone width 374a may be 25 mm while the side zone width 374b is 15 mm (e.g. 40% less than the side zone width 374a, which has the greater value).

In the embodiment depicted in FIG. 10A, the central zone may have a central zone height 376 while the side zones 373 have a side zone height 378. In the orientation shown in FIG. 10A, the heights 376, 378 may correlate to basis weights of the zones 372, 373, and in particular to basis weights of superabsorbent material 317 (and adhesives 308, 310, 334, and/or 336) within the zones 371, 373. Accordingly, in the embodiment of FIG. 10A, with the central zone height 376 being greater than the side zone heights 378, the central zone 371 may have a greater basis weight of superabsorbent material 317 (and adhesives 308, 310, 334, and/or 336) than the side zones 373. According to some embodiments of the present disclosure, the basis weight of superabsorbent material 317 within the side zones 373 may be between 0% and about 75% less than the basis weight of superabsorbent material 317 within the central zone 371. In more specific embodiments, the basis weight of superabsorbent material 317 within the side zones 373 may be between about 10% and about 70%, or between about 10% and about 60%, or between about 10% and about 50%, or between about 20% and about 60%, or between about 30% and about 60%, or between about 40% and about 60% less than the basis weight of superabsorbent material 317 within the central zone 371. As one illustrative example, the central zone 371 may have a basis weight of superabsorbent material 317 of 500 gsm, while the side zones 373 may have basis weights of superabsorbent material 317 of between about 150 gsm and about 450 gsm (using the example where the basis weight of superabsorbent material 317 within the side zones 373 is between about 10% and about 70% less than the basis weight of superabsorbent material 317 within the central zone 371).

In order to achieve the above specified differences in basis weights of superabsorbent material 317 within the central zone 371 and the side zones 373, as described previously, the cross-machine direction widths of the streams 319, 331 may differ between the absorbent material deposition stations 302a, 302b. In some embodiments, the cross-machine direction width of the stream 319 may be less than the cross-machine direction width of the stream 331. In such embodiments, the absorbent material deposition station 302a, comprising stream 319, may contribute superabsorbent material 317 substantially only within the central zone 371. Accordingly, in such embodiments, the cross-machine direction width of the stream 331 may be greater than the cross-machine direction width of the stream 319, and the absorbent material deposition station 302b, comprising stream 331, may contribute superabsorbent material 317 to both the central zone 371 and the side zones 373. Of course, in other embodiments it may be reversed where the cross-machine direction width of the stream 331 is less than the cross-machine direction width of the stream 319. Such embodiments may produce structures 101 which appear substantially similar to that depicted in FIG. 10A.

FIG. 10B depicts an exemplary cross-section of an absorbent structure 101 having a central zone 371 and side zones 373. In the embodiment of FIG. 10B, in contrast the embodiment of FIG. 10A, the central zone height 376 is less than the side zone height 378. Accordingly, in the embodiment of FIG. 10B, it is the case that the side zones 373 may have basis weight of superabsorbent material 317 that is greater than the basis weight of superabsorbent material 317 within the central zone 371. The difference in basis weights between the central zone 371 and the side zones 373 may be similar to that described with respect to FIG. 10A (for example, the basis weight of the superabsorbent material 317 of the central zone 371 may be between 0% and about 75% less than the basis weights of superabsorbent material 317 within the side zones 373).

In order to achieve the above specified differences in basis weights of superabsorbent material 317 within the central zone 371 and the side zones 373, as described previously, the cross-machine direction widths of the streams 319, 331 may differ between the absorbent material deposition stations 302a, 302b. In some embodiments, the cross-machine direction width of the stream 319 may be less than the cross-machine direction width of the stream 319. In such embodiments, the stream 319 may contribute superabsorbent material 317 substantially only within the central zone 371. Accordingly, in such embodiments, the cross-machine direction width of the stream 331 may be greater than the cross-machine direction width of the stream 319 and contribute superabsorbent material 317 to both the central zone 371 and the side zones 373. Of course, in other embodiments it may be reversed where the cross-machine direction width of the stream 331 is less than the cross-machine direction width of the stream 319. Such embodiments may produce structures 101 which appear substantially similar to that depicted in FIG. 10A.

In order to achieve the structure depicted in FIG. 10B, one of the streams 319, 331 may have a central region (in the cross-machine direction 338) that is devoid of superabsorbent material 317. In such cases, the one of the streams 319, 331 may comprise two separate, spaced apart sub-streams of superabsorbent material 317. In such embodiments, the absorbent material deposition station 302a or 302b comprising the one of the streams 319, 331 may contribute superabsorbent material 317 to only the side zones 373, while the other absorbent material deposition station 302a or 302b contributes superabsorbent material 317 to both of the central zone 371 and the side zones 373. Of course, in different embodiments it could be either of the absorbent material deposition stations 302a, 302b contributing superabsorbent material 317 to only the side zones 373 of the absorbent structure 101. According to some embodiments, the adhesive applicators 307, 309, 333 and/or 335 of the absorbent material deposition station 302a, 302b comprising the stream 319 or 331 which is split into two separate, spaced apart sub-streams, may be configured to spray adhesive into the region between the two sub-streams of the stream 319 or 331 such that the adhesives 308, 310, 334, and/or 336 used within the process 400 may generally be present throughout both the central zone 371 and the side zones 373 of the absorbent structure 101. Of course, in other embodiments, the adhesive applicators 307, 309, 333 and/or 335 of the absorbent material deposition station 302a, 302b comprising the stream 319 or 331 which is split into two separate, spaced apart sub-streams, may be configured to spray adhesive only in the region of the sub-streams of the stream 319 or 331 such that the adhesives 308 and/or 310, or 334 and/or 336 may generally be absent in the central zone 371 of the absorbent structure 101. Still further, it may be the case that the basis weights of the side zones 373 may not be equal to each other. However, in most embodiments the basis weights of the side zones 373 may not differ from each other by more than 50%.

In the embodiments of absorbent structures 101 of FIGS. 10A-10B, it may be particularly preferred for the mixture 320 to comprise superabsorbent material 317 in the regions of higher basis weights in amounts between about 100 gsm and about 700 gsm, or more particularly between about 150 gsm and about 700 gsm, or between about 200 gsm and about 700 gsm, or between about 250 gsm and about 700 gsm, or between about 250 gsm and about 600 gsm, or between about 250 gsm and about 550 gsm. In any of these such embodiments, the adhesives 308 and/or 310 may combine to equal an add-on percentage of between about 2% and about 7%, or more particularly between about 2% and about 6%, or between about 2% and about 5.5%, or between about 2% and about 5%, or between about 2% and about 4.5%, or between about 2.5% and about 4.5%, or between about 2.5% and about 4%. It may be particularly advantageous for the adhesives 308 and/or 310 to combine to equal an add-on percentage of less than about 5%, less than about 4.5%, or less than about 4%, or less than about 3.5%, or less than about 3%, or less than about 2.5%.

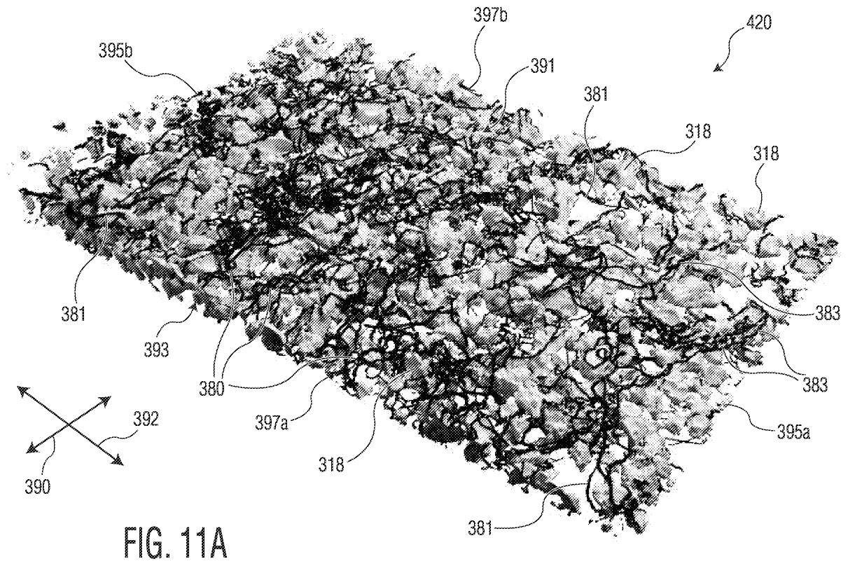

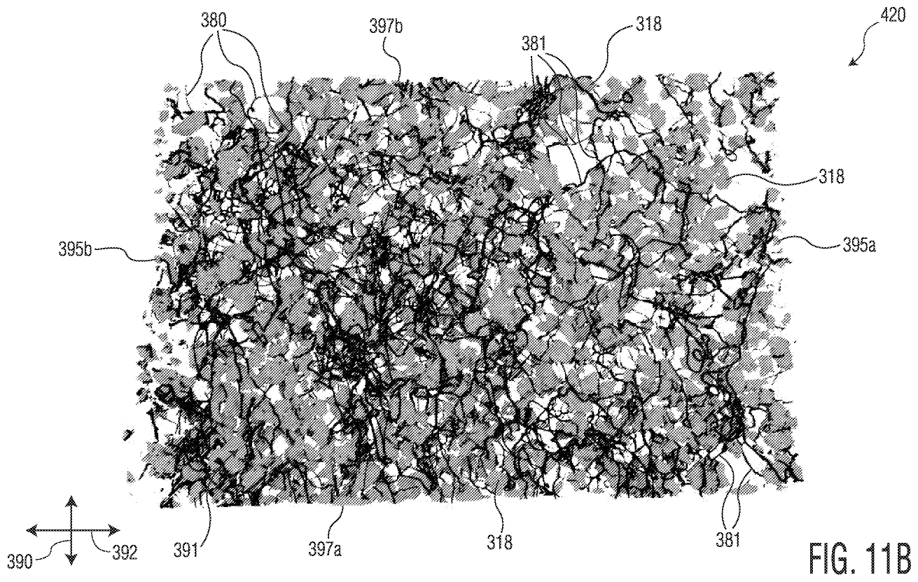

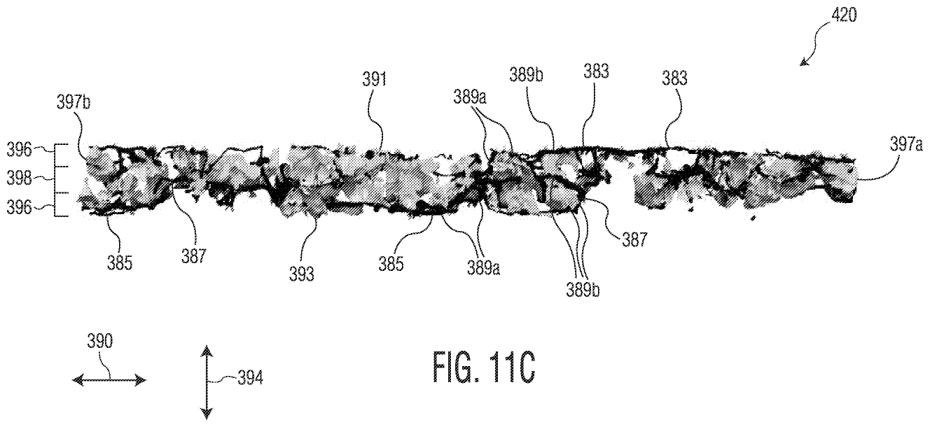

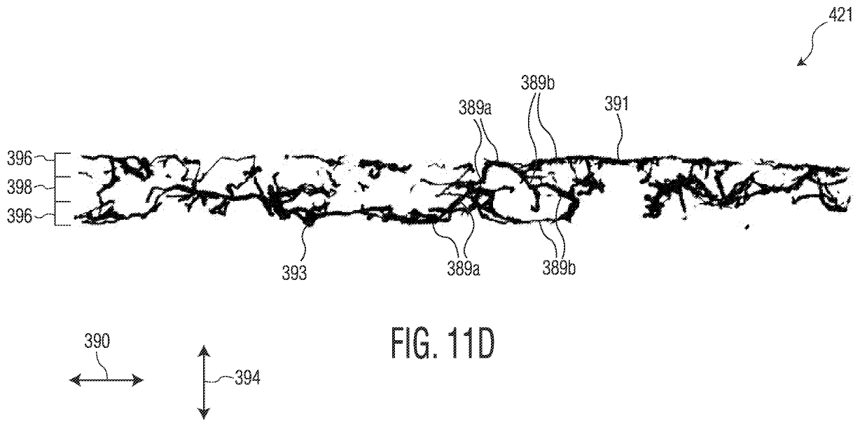

FIG. 11A is a perspective view of a computer-generated image 420 of a deposited mixture 320 which is based on micro-CT images taken from an exemplary deposited mixture 320 formed by the process 300. More specifically, the mixture 320 used to generate the computer-generated mixture 420 shown in FIG. 11A was formed by process 300, including the details of the absorbent deposition station 302 of FIG. 7, where the stream 319 and the adhesives 308 and 310 were configured to be same as listed for the first exemplary absorbent structures detailed below, and the resulting mixture 320 had superabsorbent material 317 disposed in an amount of 400 gsm and where the adhesives 308, 310, 334, and 336 were present at an add-on rate of 5%. The mixture 320 was stained with osmium tetroxide and then micro-CT scanning was performed, both according to standard, known techniques for staining and scanning. As part of the micro-CT process, a portion of stained deposited mixture 320 chosen approximately from the center of the mixture 320 (e.g. structure 101) in the widthwise and lengthwise directions was chosen for imaging. The portion had dimensions of approximately 3 cm by 1 cm and was sliced into approximately one-thousand two-hundred and fifty individual segments extending in the lateral direction 392, each segment extending from end edge 395a to end edge 395b and comprising 1986 pixels in the longitudinal dimension (e.g. along longitudinal direction 392). Each segment further comprised 504 pixels in the vertical direction 394 between the first surface 391 and the second surface 393. A voxel size of 8.0 micrometers was used. From the captured segments, a three-dimensional model was generated and is depicted in FIGS. 11A-11D.

The adhesive filaments 316 which were sprayed by the applicators 307, 309, 311 and/or 313 cross and connect as the adhesives 308, 310, 334, and/or 336 and the superabsorbent material 317 intermix to form a three-dimensional mesh network 380 having network adhesive filaments 381 extending substantially throughout the three-dimensional space formed by image 420, as can be seen in FIGS. 11A and 11B. The network adhesive filaments 381 extend or are oriented in a substantially random manner throughout the three-dimensional mesh network 380—likely due to the random, turbulent nature of the mixing of the adhesives 308, 310, 334, and/or 336 and the superabsorbent material 317 prior to deposition. As used herein, the network adhesive filaments 381 may be considered to extend substantially throughout the three-dimensional space formed by the deposited mixture of the image 420 where the network adhesive filaments 381 extend between and intermingle with a majority, or super-majority, of the individual superabsorbent particles 318. Such a configuration is in contrast to configurations where adhesive filaments extend over pockets or groupings of superabsorbent particles and do not extend into and between individual superabsorbent particles 318 of the pocket or grouping of superabsorbent particles 318 and/or where adhesive filaments are applied to deposited superabsorbent material 317—particularly where the adhesive filaments are applied in a pattern (e.g. a spray, swirl, line pattern, or the like). The superabsorbent material 317 are also disposed throughout the three-dimensional mesh network 380, shown as particles 318, and are immobilized by contact with one or more of the network adhesive filaments 381.

The processes 300 and 400 may operate to intermix the adhesives 308, 310, 334, and/or 336 with the superabsorbent material 317 to such a degree that the network adhesive filaments 381 contact substantially all of the individual superabsorbent material 317. The network adhesive filaments 381 may wrap around a majority, or a super-majority, of the individual superabsorbent particles 318. As used herein, the network adhesive filaments 381 may be considered to wrap around an individual superabsorbent particle 318 if combined lengths of individual network adhesive filaments 381 in contact with an individual superabsorbent particle 318 equal at least 40% of a maximum circumference of the individual superabsorbent particle 318.

As seen in both FIG. 11A and FIG. 11B, which is a top plan view of a portion of the image 420 of FIG. 11A, along with FIG. 11C, the image 420 of the deposited mixture may generally have a first surface 391 and a second surface 393 disposed opposite the first surface 391, along with end edges 395a, 395b and side edges 397a, 397b. Each of the first surface 391 and the second surface 393 extend generally in the lateral and longitudinal directions 390, 392. At each of the first surface 391 and the second surface 393, the mesh network 380 may comprise network adhesive filaments 381 which extend substantially in the lateral and longitudinal directions 390, 392. For example, first network adhesive filaments 383 can be seen extending substantially in the lateral and longitudinal directions 390, 392 along the first surface 391. Second network adhesive filaments 385 (shown in FIG. 11C) may extend substantially in the lateral and longitudinal directions 390, 392 along the second surface 393.

The network adhesive filaments 381 of the three-dimensional mesh network 380 may further include vertically extending filaments 387 which can be seen extending in the vertical direction 394 in FIG. 11C. FIG. 11C represents a laterally extending slice of the image 420 of FIG. 11A and having a length in the longitudinal direction 392 of 0.5 mm, showing in more detail interaction of the particles 318 and adhesive filaments 381. FIG. 11D is the same image as FIG. 11C with the particles 318 removed to show in more detail the adhesive filaments 381 and their disposition through the vertical direction 394.

At least some of these vertically extending filaments 387 extend all the way from the first surface 391 to the second surface 393 and connect the first network adhesive filaments 383 to the second network adhesive filaments 385 to form the three-dimensional mesh network 380. Of course, as can be seen, the vertically extending filaments 387 may not extend perfectly in the vertical direction 394 and may twist and turn between and around individual superabsorbent particles 318 such that at least some of the vertically extending filaments 387 extend also in the lateral and/or longitudinal directions 390, 392. In at least some embodiments, individual network adhesive filaments 381 may themselves extend from along part of the first surface 391 (for example in the longitudinal and/or lateral directions 390, 392), transition to extending in the vertical direction 394, and then connect with the second surface 392—possibly further extending along the longitudinal and/or lateral directions 390, 392 at the second surface 392. Such behavior can be seen with respect to network adhesive filaments 389a and 389b.

Another feature that can be seen to some extent in the FIGS. 11C-11D is the relative distribution of the network adhesive filaments 381 within different vertical regions of the deposited mixture represented by the image 420. For example, as indicated in FIGS. 11C and 11D, the image 420 may be split into exterior regions 396 and an interior region 398 disposed between the exterior regions 396, spanning the vertical direction 394. The exterior regions 396 may each be defined by a thickness of 33% of the overall thickness of the structure 420, while the interior region 398 may be defined by a thickness of 33% of the overall thickness of the structure 420.

It has been found that the processes 300 and/or 400 may desirably penetrate the adhesives 308, 310, 334, and/or 336 into the interior region 398, thereby promoting high superabsorbent particle capture values and greater pad uniformity through greater evenness of distribution of the superabsorbent material 317 and adhesives 308, 310, 334, and/or 336 throughout the structure 420. This is particularly true where the formed mixtures 320 of the present disclosure have basis weights of superabsorbent material 317 greater than 300 gsm, or greater than 400 gsm, or greater than 500 gsm, or greater than 600 gsm, or greater than 700 gsm. As the desired basis weights of superabsorbent material 317 in a mixture 320 increases, penetration of the adhesives 308, 310, 334, and/or 336 into an interior of a streams 319 and/or 331 becomes more difficult and where the processes 300 and/or 400 excel in comparison to prior art processes.

In order to assess the ability of the processes 300 and/or 400 to penetrate adhesive into the interior region 398 of the formed mixtures 320, analysis of two sample codes was performed. In the analysis, two sample codes were generated according to process 400 having a basis weight of superabsorbent material of 500 gsm and adhesive disposed in an add-on amount of 5%. From these two sample codes, micro-CT images of portions of the codes were formed—according to the standard processes and techniques mentioned above. The Adhesive Distribution Test Method, described in detail below, was then performed on the generated micro-CT images to determine the relative quantity of adhesive located within the interior region 398 of the imaged portion of the two sample codes. The micro-CT images were generated using the known staining and imaging methods described above.

According to the Adhesive Distribution Test Method, it was found that the first sample code had 28.0% of the of the total amount of adhesive within the first sample code located within the interior region 398 of the first sample code, with the standard deviation being 8.4%. The second sample code was found to have 30.7% of the total amount of adhesive within the second sample code located within the interior region 398 of the second sample code, with the standard deviation being 8.9%. Accordingly, mixtures 320 formed according to the processes 300 and/or 400 can cause greater than 28% of the total amount of adhesive within the mixture 320 to be located within the interior region 398, or greater than 30.5% of the total amount of adhesive within a mixture 320 to be located within the interior region 398. However, in further potential embodiments, it is believed that greater than 33%, or even greater than 35% of the total amount of adhesive within a mixture 320 being located within the interior region 398 of the mixture 320 can be achieved through slight modifications to the processes 300 and/or 400—for example in terms of adhesive add-on amount, vacuum energy, nip pressure, location and angle of the nozzles, and the like. Such high adhesive penetration into the interior region 398 of the formed mixtures of the processes 300 and/or 400 result help to drive improved absorbent structure properties including the capture and stabilization of the superabsorbent particles (e.g. resulting in less ‘free’ particles), pad integrity—particularly wet pad integrity—and even a relative uniformity of the distribution of the superabsorbent particles within the absorbent structures 101.

The absorbent structures 101 produced by the process 300 and/or 400 have been shown to have beneficial characteristics with respect to prior art absorbent bodies. For example, the processes 300 and/or 400 have been shown to produce absorbent structures 101 providing superior performance with respect to the capture and immobilization of superabsorbent material 317, as well as a superior fluid intake ability, particularly with respect to 3^{rd} intake times, as will be described in more detail below.

In order to compare absorbent bodies, a number of different absorbent structures 101 were formed by the described processes 300 and/or 400 and tested with respect to absorbent bodies formed by prior art processes. As will be described below, exemplary absorbent structures 101 and exemplary prior art absorbent bodies were compared with respect to the SAM Capture Test Method, the Wet Pad Integrity Test Method, and the Pad Uniformity Test Method, as described herein below, to produce comparative results.

### First Exemplary Absorbent Structures

First exemplary absorbent structures 101, labeled as absorbent bodies S23, S27, S53, and S57, as described herein, were formed according to the exemplary process 300. Specifically, first exemplary absorbent structures 101 were formed according to the exemplary process 300 having basis weights of 200 gsm with an adhesive add-on of 3% (labeled as structures S23), having basis weights of 200 gsm with an adhesive add-on of 7% (labeled as structures S27), having basis weights of 500 gsm with an adhesive add-on of 3% (labeled as structures S53), and having basis weights of 500 gsm with an adhesive add-on of 7% (labeled as structures S57).

The settings of the process 300 used to form the exemplary absorbent structures S23, S27, S53, and S57 include using both adhesive applicators 307, 309 with adhesive applicator 307 positioned a distance from the web material 303 and the stream 319 such that the adhesive contacted the stream 319 a distance of 6.4 mm from the web material 303 (e.g. the distance 361). The adhesive applicator 309 was positioned a distance from the web material 303 and the stream 319 such that the adhesive 310 contacted the stream 319 a distance of 16 mm from the web material 303 (e.g. the distance 367 plus the distance 361). Additionally, the chute 315 was positioned a distance 359 of 76 mm from the web material 303. The adhesive nozzle 321 was positioned at an angle 359a of 60 degrees with respect to the machine direction 330, and the adhesive nozzle 323 was also positioned at an angle of 60 degrees with respect to the machine direction 330. The nozzles 321 and 232 were Universal™ Signature™ Spray Nozzles available from the Nordson Corporation. The chute width 356 was set at 12 mm, and a nip pressure at nip station 327 was 1 PLI (175.1 N/m). An 8 gsm SMS material was used for material web materials 303 and 324. Vacuum energy was applied such that the forming surface had a pressure differential of approximately 0.51 m of water.

### Second Exemplary Absorbent Structures

Second exemplary absorbent structures 101, labeled as absorbent bodies D23-D67, as described herein, were formed according to the exemplary process 400. Specifically, second exemplary absorbent structures 101 were formed according to the exemplary process 400 having basis weights of 200 gsm with an adhesive add-on of 3% (labeled as structures D23), having basis weights of 200 gsm with an adhesive add-on of 4% (labeled as structures D24), having basis weights of 200 gsm with an adhesive add-on of 5% (labeled as structures D25), having basis weights of 200 gsm with an adhesive add-on of 6% (labeled as structures D26), and having basis weights of 200 gsm with an adhesive add-on of 7% (labeled as structures D27). Further second exemplary absorbent structures 101 were formed according to the exemplary process 400 having basis weights of 300 gsm with an adhesive add-on of 3% (labeled as structures D33), having basis weights of 300 gsm with an adhesive add-on of 4% (labeled as structures D34), having basis weights of 300 gsm with an adhesive add-on of 5% (labeled as structures D35), having basis weights of 300 gsm with an adhesive add-on of 6% (labeled as structures D36), and having basis weights of 300 gsm with an adhesive add-on of 7% (labeled as structures D37). Still more second exemplary absorbent structures 101 were formed according to the exemplary process 400 having basis weights of 400 gsm with an adhesive add-on of 3% (labeled as structures D43), having basis weights of 400 gsm with an adhesive add-on of 4% (labeled as structures D44), having basis weights of 400 gsm with an adhesive add-on of 5% (labeled as structures D45), having basis weights of 400 gsm with an adhesive add-on of 6% (labeled as structures D46), and having basis weights of 400 gsm with an adhesive add-on of 7% (labeled as structures D47). Even more second exemplary absorbent structures 101 were formed according to the exemplary process 400 having basis weights of 500 gsm with an adhesive add-on of 3% (labeled as structures D53), having basis weights of 500 gsm with an adhesive add-on of 4% (labeled as structures D54), having basis weights of 500 gsm with an adhesive add-on of 5% (labeled as structures D55), having basis weights of 500 gsm with an adhesive add-on of 6% (labeled as structures D56), and having basis weights of 500 gsm with an adhesive add-on of 7% (labeled as structures D57). Further second exemplary absorbent structures 101 were formed according to the exemplary process 400 having basis weights of 600 gsm with an adhesive add-on of 2% (labeled as structures D62), having basis weights of 600 gsm with an adhesive add-on of 3% (labeled as structures D63), having basis weights of 600 gsm with an adhesive add-on of 4% (labeled as structures D64), having basis weights of 600 gsm with an adhesive add-on of 5% (labeled as structures D65), having basis weights of 600 gsm with an adhesive add-on of 6% (labeled as structures D66), and having basis weights of 600 gsm with an adhesive add-on of 7% (labeled as structures D67).

The settings of the process 400 used to form the exemplary absorbent structures D23-D27, D33-D37, D43-D47, D53-D57, and D62-D67 included using both adhesive applicators 307, 309 within absorbent material deposition stations 302a. The adhesive applicator 307 was positioned a distance from the web material 303 and the stream 319 such that the adhesive contacted the stream 319 a distance of 6.4 mm from the web material 303 (e.g. the distance 361). The adhesive applicator 309 was positioned a distance from the web material 303 and the stream 319 such that the adhesive 310 contacted the stream 319 a distance of 16 mm from the web material 303 (e.g. the distance 367 plus the distance 361). Additionally, the chute 315 was positioned a distance 359 of 76 mm from the web material 303. The adhesive nozzle 321 was positioned at an angle 359a of 60 degrees with respect to the machine direction 330, and the adhesive nozzle 323 was also positioned at an angle of 60 degrees with respect to the machine direction 330. The chute width 356 was set at 12 mm, and a nip pressure at nip station 327 was 1 PLI (175.1 N/m). The settings for absorbent material deposition station 302b were substantially the same as above for absorbent material deposition station 302a. An 8 gsm SMS material was used for material web materials 303 and 324, and vacuum energy was applied such that the forming surface had a pressure differential of approximately 0.51 m of water.

### Third Exemplary Absorbent Structures

Third exemplary absorbent structures 101, labeled as absorbent structures N23-N67 (or, more specifically absorbent structures N23-N27, N33-N37, N43-N47, N53-N57, and N62-N67), as described herein, were formed according to an exemplary prior art process according to prior art document U.S. Pat. No. 8,986,474 to Kufner et al., and assigned to Nordson Corporation (hereinafter “Nordson”, or the “Nordson process”, or the “Nordson reference”). The exemplary absorbent structures N23-N67 were formed according to the Nordson process according to FIG. 3 of U.S. Pat. No. 8,986,474, where a single absorbent material deposition station was employed with two adhesive dispensing units. Such dispensing units, for example as depicted as units 22, 72 of FIG. 3 of the Nordson reference, were configured such that the discharged adhesive streams 26, 76 converged at the powder mixture 56 and each were oriented at angles of 45 degrees. The adhesive streams 26, 76 both contacted that powder mixture 56 a distance 12.7 mm from the web facing material. A chute similar to chute 315 was used and was placed 76 mm away from the web facing material and was set to have a width (e.g. similar to width 356 of the chute 315 of the present disclosure) of 12 mm. Although not necessarily disclosed in the Nordson reference, the absorbent structures formed according to the Nordson process were subjected to the same post processing as described with respect to processes 300 and 400, namely passing through a nip station such as nip station 327 at a setting of 1 PLI (175.1 N/m) and then being cut into individual absorbent structures 101. Vacuum energy was applied such that the forming surface had a pressure differential of approximately 0.51 m of water. As with the first and second exemplary absorbent bodies, an 8 gsm SMS material was used for material web materials 303 and 324.

With the prior art Nordson process set up as described above, a number of absorbent structures were produced. Specifically, third absorbent structures 101 having basis weights of 200 gsm with an adhesive add-on of 3% (labeled as structures N23), having basis weights of 200 gsm with an adhesive add-on of 4% (labeled as structures N24), having basis weights of 200 gsm with an adhesive add-on of 5% (labeled as structures N25), having basis weights of 200 gsm with an adhesive add-on of 6% (labeled as structures N26), and having basis weights of 200 gsm with an adhesive add-on of 7% (labeled as structures N27) were produced. Further third exemplary absorbent structures were formed according to the exemplary Nordson process having basis weights of 300 gsm with an adhesive add-on of 3% (labeled as structures N33), having basis weights of 300 gsm with an adhesive add-on of 4% (labeled as structures N34), having basis weights of 300 gsm with an adhesive add-on of 5% (labeled as structures N35), having basis weights of 300 gsm with an adhesive add-on of 6% (labeled as structures N36), and having basis weights of 300 gsm with an adhesive add-on of 7% (labeled as structures N37). Still more third exemplary absorbent structures were formed according to the Nordson process having basis weights of 400 gsm with an adhesive add-on of 3% (labeled as structures N43), having basis weights of 400 gsm with an adhesive add-on of 4% (labeled as structures N44), having basis weights of 400 gsm with an adhesive add-on of 5% (labeled as structures N45), having basis weights of 400 gsm with an adhesive add-on of 6% (labeled as structures N46), and having basis weights of 400 gsm with an adhesive add-on of 7% (labeled as structures N47). Even more third exemplary absorbent structures were formed according to the Nordson process having basis weights of 500 gsm with an adhesive add-on of 3% (labeled as structures N53), having basis weights of 500 gsm with an adhesive add-on of 4% (labeled as structures N54), having basis weights of 500 gsm with an adhesive add-on of 5% (labeled as structures N55), having basis weights of 500 gsm with an adhesive add-on of 6% (labeled as structures N56), and having basis weights of 500 gsm with an adhesive add-on of 7% (labeled as structures N57). Further third exemplary absorbent structures 101 were formed having basis weights of 600 gsm with an adhesive add-on of 2% (labeled as structures N62), having basis weights of 600 gsm with an adhesive add-on of 3% (labeled as structures N63), having basis weights of 600 gsm with an adhesive add-on of 4% (labeled as structures N64), having basis weights of 600 gsm with an adhesive add-on of 5% (labeled as structures N65), having basis weights of 600 gsm with an adhesive add-on of 6% (labeled as structures N66), and having basis weights of 600 gsm with an adhesive add-on of 7% (labeled as structures N67).

### SAM Capture Test Method Results

The absorbent structures 101, labeled as absorbent structures S23, S27, S53, and S57, absorbent structures D23-D27, D33-D37, D43-D47, D53-D57, and D62-D67, and absorbent structures N23-N27, N33-N37, N43-N47, N53-N57, and N62-N67 below, were all tested according to the SAM Capture Test Method described in more detail below. Five samples were tested for each code and the averaged results for each code are displayed below in Tables 1A-1L.

The SAM gsm and the % Adh columns indicate the process settings used to form the corresponding structures. For example, the SAM gsm column indicates that the process was set up to produce absorbent structures 101 having an average basis weight of superabsorbent particles 317 of 200 gsm. The % Adh column indicates that the process was set up to produce absorbent structures 101 having a combined average basis weight of the one or more adhesives used of the specified percent, by weight, of the weight of the superabsorbent particles 317 of the structure 101. As one specific example, where the SAM gsm column indicates 200 gsm and the % Adh column indicates 3%, the specified absorbent structures 101 was formed to have a basis weight of adhesive(s) that is 3% of the 200 gsm of superabsorbent particles 317—6 gsm—disposed throughout the structure 101. The Avg % SAM capture value is a measure of the percentage of superabsorbent particles 317 retained by the specified absorbent structure 101 at the end of the SAM Capture Test Method.

TABLE 1A

| | SAM | | Avg Pre- | Avg Post- | Avg SAM | Avg % SAM |
|---|---|---|---|---|---|---|
| Code | gsm | % Adh | Wt (g) | Wt (g) | Loss (g) | Capture |
| S23 | 200 | 3 | 8.41 | 6.87 | 1.54 | 82.0% |
| S27 | 200 | 7 | 8.82 | 8.40 | 0.42 | 95.2% |

TABLE 1B

| | SAM | | Avg Pre- | Avg Post- | Avg SAM | Avg % SAM |
|---|---|---|---|---|---|---|
| Code | gsm | % Adh | Wt (g) | Wt (g) | Loss (g) | Capture |
| S53 | 500 | 3 | 18.4 | 8.78 | 9.61 | 47.7% |
| S57 | 500 | 7 | 19.5 | 15.6 | 3.9 | 80.0% |

TABLE 1C

| | SAM | | Avg Pre- | Avg Post- | Avg SAM | Avg % SAM |
|---|---|---|---|---|---|---|
| Code | gsm | % Adh | Wt (g) | Wt (g) | Loss (g) | Capture |
| D23 | 200 | 3 | 8.69 | 8.68 | .01 | 99.9% |
| D24 | 200 | 4 | 8.76 | 8.76 | 0 | 100.0% |
| D25 | 200 | 5 | 8.64 | 8.64 | 0 | 100.0% |
| D26 | 200 | 6 | 8.88 | 8.88 | 0 | 100.0% |
| D27 | 200 | 7 | 8.79 | 8.79 | 0 | 100.0% |

TABLE 1D

| | SAM | | Avg Pre- | Avg Post- | Avg SAM | Avg % SAM |
|---|---|---|---|---|---|---|
| Code | gsm | % Adh | Wt (g) | Wt (g) | Loss (g) | Capture |
| N23 | 200 | 3 | 8.64 | 8.64 | 0 | 100.0% |
| N24 | 200 | 4 | 8.63 | 8.63 | 0 | 100.0% |
| N25 | 200 | 5 | 8.72 | 8.70 | 0.02 | 99.8% |
| N26 | 200 | 6 | 8.61 | 8.60 | 0.01 | 99.9% |
| N27 | 200 | 7 | 8.83 | 8.82 | 0.01 | 99.9% |

TABLE 1E

| | SAM | | Avg Pre- | Avg Post- | Avg SAM | Avg % SAM |
|---|---|---|---|---|---|---|
| Code | gsm | % Adh | Wt (g) | Wt (g) | Loss (g) | Capture |
| D33 | 300 | 3 | 12.31 | 12.30 | 0.01 | 99.9% |
| D34 | 300 | 4 | 12.50 | 12.43 | 0.07 | 99.6% |
| D35 | 300 | 5 | 12.56 | 12.55 | 0.01 | 99.9% |
| D36 | 300 | 6 | 12.80 | 12.78 | 0.02 | 99.9% |
| D37 | 300 | 7 | 12.85 | 12.85 | 0 | 100.0% |

TABLE 1F

| | SAM | % | Avg Pre- | Avg Post- | Avg SAM | Avg % SAM |
|---|---|---|---|---|---|---|
| Code | gsm | Adh | Wt (g) | Wt (g) | Loss (g) | Capture |
| N33 | 300 | 3 | 11.77 | 11.66 | 0.11 | 99.1% |
| N34 | 300 | 4 | 12.03 | 11.91 | 0.12 | 99.0% |
| N35 | 300 | 5 | 12.28 | 12.22 | 0.06 | 99.5% |
| N36 | 300 | 6 | 12.52 | 12.46 | 0.06 | 99.5% |
| N37 | 300 | 7 | 12.68 | 12.61 | 0.07 | 99.4% |

TABLE 1G

| | SAM | % | Avg Pre- | Avg Post- | Avg SAM | Avg % SAM |
|---|---|---|---|---|---|---|
| Code | gsm | Adh | Wt (g) | Wt (g) | Loss (g) | Capture |
| D43 | 400 | 3 | 16.1 | 15.86 | 0.24 | 98.5% |
| D44 | 400 | 4 | 16.24 | 16.12 | 0.12 | 99.3% |
| D45 | 400 | 5 | 16.61 | 16.57 | 0.04 | 99.8% |
| D46 | 400 | 6 | 16.77 | 16.73 | 0.04 | 99.7% |
| D47 | 400 | 7 | 17.13 | 17.08 | 0.05 | 99.7% |

TABLE 1H

| | SAM | % | Avg Pre- | Avg Post- | Avg SAM | Avg % SAM |
|---|---|---|---|---|---|---|
| Code | gsm | Adh | Wt (g) | Wt (g) | Loss (g) | Capture |
| N43 | 400 | 3 | 15.80 | 15.29 | 0.51 | 96.8% |
| N44 | 400 | 4 | 16.16 | 15.72 | 0.44 | 97.3% |
| N45 | 400 | 5 | 16.26 | 15.91 | 0.35 | 97.9% |
| N46 | 400 | 6 | 16.50 | 16.28 | 0.22 | 98.7% |
| N47 | 400 | 7 | 16.67 | 16.48 | 0.19 | 98.8% |

TABLE 1I

| | SAM | % | Avg Pre- | Avg Post- | Avg SAM | Avg % SAM |
|---|---|---|---|---|---|---|
| Code | gsm | Adh | Wt (g) | Wt (g) | Loss (g) | Capture |
| D53 | 500 | 3 | 19.75 | 19.19 | 0.56 | 97.2% |
| D54 | 500 | 4 | 19.50 | 19.37 | 0.13 | 99.3% |
| D55 | 500 | 5 | 19.69 | 19.60 | 0.09 | 99.5% |
| D56 | 500 | 6 | 19.56 | 19.51 | 0.05 | 99.8% |
| D57 | 500 | 7 | 19.81 | 19.78 | 0.03 | 99.8% |

TABLE 1J

| | SAM | % | Avg Pre- | Avg Post- | Avg SAM | Avg % SAM |
|---|---|---|---|---|---|---|
| Code | gsm | Adh | Wt (g) | Wt (g) | Loss (g) | Capture |
| N53 | 500 | 3 | 19.48 | 18.07 | 1.41 | 92.8% |
| N54 | 500 | 4 | 19.57 | 18.55 | 1.02 | 94.8% |
| N55 | 500 | 5 | 19.94 | 19.18 | 0.76 | 96.2% |
| N56 | 500 | 6 | 19.98 | 19.33 | 0.65 | 96.7% |
| N57 | 500 | 7 | 20.42 | 19.67 | 0.75 | 96.3% |

TABLE 1K

| | SAM | % | Avg Pre- | Avg Post- | Avg SAM | Avg % SAM |
|---|---|---|---|---|---|---|
| Code | gsm | Adh | Wt (g) | Wt (g) | Loss (g) | Capture |
| D62 | 600 | 2 | 23.50 | 22.42 | 1.08 | 95.4% |
| D63 | 600 | 3 | 23.83 | 22.78 | 1.05 | 95.6% |
| D64 | 600 | 4 | 23.80 | 23.39 | 0.41 | 98.3% |
| D65 | 600 | 5 | 24.01 | 23.57 | 0.44 | 98.1% |
| D66 | 600 | 6 | 23.97 | 23.71 | 0.26 | 98.9% |
| D67 | 600 | 7 | 24.04 | 23.88 | 0.16 | 99.3% |

TABLE 1L

| | SAM | % | Avg Pre- | Avg Post- | Avg SAM | Avg % SAM |
|---|---|---|---|---|---|---|
| Code | gsm | Adh | Wt (g) | Wt (g) | Loss (g) | Capture |
| N62 | 600 | 2 | 23.53 | 18.21 | 5.32 | 77.4% |
| N63 | 600 | 3 | 23.98 | 21.12 | 2.86 | 88.1% |
| N64 | 600 | 4 | 24.37 | 21.63 | 2.74 | 88.8% |
| N65 | 600 | 5 | 23.90 | 20.79 | 3.11 | 87.0% |
| N66 | 600 | 6 | 24.26 | 20.78 | 3.48 | 85.6% |
| N67 | 600 | 7 | 23.85 | 21.29 | 2.56 | 89.3% |

Accordingly, there are clear differences in the performance of some of the codes formed according to aspects of the present disclosure and codes produced by the Nordson process, particularly codes with relatively lower % Adh values. Specifically, it can be seen that absorbent structures 101 produced according to aspects of the present disclosure which have basis weights of superabsorbent particles 317 of between 400 gsm and 600 gsm and % Adh values of between 4% and 5%, have % SAM Capture Values of greater than 98.0, which is higher than any of the codes of absorbent structures produced by the Nordson process (code N45, falling within the specified ranges for SAM gsm and % Adh, has the highest % SAM Capture Value at 97.9). Alternatively, the structures 101 formed according to aspects of the present disclosure which have basis weights of superabsorbent particles 317 of between 400 gsm and 600 gsm and % Adh values of between 4% and 5% can be described as having % SAM Capture Values of greater than 98.5.

Further, many of the codes produced according to aspects of the present disclosure have % SAM Capture Values greater than 98.0, such as codes D65 (% SAM Capture Value of 98.1), D64 (% SAM Capture Value of 98.3), D55 (% SAM Capture Value of 99.5), D54 (% SAM Capture Value of 99.3), D45 (% SAM Capture Value of 99.8), and D44 (% SAM Capture Value of 99.3). Many of the corresponding codes of absorbent structures produced according to the Nordson process (e.g. codes having corresponding SAM gsm and % Adh values) have much lower % SAM Capture Values—for example, N65 has a % SAM Capture Value of 87.0, N64 has a % SAM Capture Value of 88.8, and N44 has a % SAM Capture Value of 97.3.

With further emphasis on the codes having basis weights of between 500 gsm and 600 gsm, having % Adh values of between 4% and 5%, the absorbent structures 101 formed according to aspects of the present disclosure all have % SAM Capture Values greater than 96.5. For instance, the codes D54, D55, D64, and D65 have % SAM Capture Values of 99.3, 99.5, 98.3, and 98.1, respectively. The corresponding codes N54, N55, N64, and N65 have % SAM Capture Values of 94.8, 96.2, 88.8, and 87.0, respectively.

Advantages in performance of codes of structures 101 formed according to aspects of the present disclosure in comparison to codes of absorbent structures produced by the Nordson process may also be evident where the basis weights of superabsorbent particles 317 are between 500 gsm and 600 gsm and the % Adh values are between 3% and 4%. In such examples, the structures 101 produced according to aspects of the present disclosure all have % SAM Capture Values of greater than 95.0, which is higher than any of the codes of absorbent structures produced by the Nordson process (code N54, falling within the specified ranges for SAM gsm and % Adh, has the highest % SAM Capture Value at 94.8).

Further, many of the codes produced according to aspects of the present disclosure have % SAM Capture Values greater than 95.0, such as codes D53 (% SAM Capture Value of 97.2), D54 (% SAM Capture Value of 99.3), D63 (% SAM Capture Value of 95.6), and D64 (% SAM Capture Value of 98.3). Many of the corresponding codes of absorbent structures produced according to the Nordson process have much lower % SAM Capture Values—for example, N53 has a % SAM Capture Value of 92.8, N63 has a % SAM Capture Value of 88.1, and N64 has a % SAM Capture Value of 88.8.

Where the % Adh value is increased to be between 4% and 5%, the structures 101 produced according to aspects of the present disclosure having basis weights of superabsorbent particles 317 of between 500 gsm and 600 gsm, still outperform the absorbent structures produced by the Nordson process by all having % SAM Capture Values of at greater than 97.0. For example, codes D54, D55, D64, and D65 have % SAM Capture Values of 99.3, 99.5, 98.3, and 98.1, respectively. The corresponding absorbent structures produced by the Nordson process, codes N54, N55, N64, and N65 have % SAM Capture Values of 94.8, 96.2, 88.8, and 87.0, respectively.

Even where the % Adh value is increased to be between 5% and 6%, the structures 101 produced according to aspects of the present disclosure, and having basis weights of superabsorbent particles 317 of between 500 gsm and 600 gsm, still outperform the absorbent structures produced by the Nordson process by all having % SAM Capture Values of at greater than 97.0. For example, codes D55, D56, D65, and D66 have % SAM Capture Values of 99.5, 99.8, 98.1, and 98.9, respectively. The corresponding absorbent structures produced by the Nordson process, codes N55, N56, N65, and N66 have % SAM Capture Values of 96.2, 96.7, 87.0, and 85.6, respectively.

### Wet Pad Integrity Test Method Results

In obtaining comparative measurements of absorbent structures 101 formed according to aspects of the present disclosure and absorbent structures formed according to the Nordson process, a number of different codes were produced. As shown in the TABLE 2A below, absorbent structures 101 formed according to aspects of the present disclosure, labeled as codes DD23, DD27, DD53, and DD57 were produced. The codes DD23, DD27, DD53, and DD57 were formed by a process similar to that described above with respect to the Second Exemplary Absorbent Structures. Additionally, corresponding absorbent structures were formed according to the Nordson process, shown in TABLE 2B and labeled as NN23, NN27, NN53, and NN57. The codes NN23, NN27, NN53, and NN57 were formed by a process similar to that described above with respect to the Third Exemplary Absorbent Structures. Five of each of these codes were tested according to the Wet Pad Integrity Test Method described in more detail below, and the results are shown in TABLES 2A and 2B below. The Avg #column details the average number of shakes (average of the five samples tested) imparted to the structures during Wet Pad Integrity Test Method for which the structures maintained their integrity, capped at 50 shakes.

TABLE 2A

| | SAM | | |
|---|---|---|---|
| Code | gsm | % Adh | Avg # |
| DD23 | 200 | 3 | 20 |
| DD27 | 200 | 7 | 50 |
| DD53 | 500 | 3 | 3 |
| DD57 | 500 | 7 | 38 |

TABLE 2B

| | SAM | | |
|---|---|---|---|
| Code | gsm | % Adh | Avg # |
| NN23 | 200 | 3 | 19 |
| NN27 | 200 | 7 | 50 |
| NN53 | 500 | 3 | 0 |
| NN57 | 500 | 7 | 18 |

As can be seen in TABLES 2A and 2B, there are clear benefits in terms of Wet Pad Integrity to the absorbent structures 101 formed according to aspects of the present disclosure in comparison to the absorbent bodies formed according to the Nordson process. For example, the code DD57, representing an absorbent structure 101 formed to have an average basis weight of superabsorbent material 317 of 500 gsm and a combined basis weight of one or more adhesives of 7% of the basis weight of the superabsorbent material 317, had a Wet Pad Integrity value of 38, which is 110% higher than the Wet Pad Integrity value for the corresponding code NN57 formed according to the Nordson process (Wet Pad Integrity value for NN57 is 18). In other embodiments, the absorbent structures 101 formed according to aspects of the present disclosure may be described as having Wet Pad Integrity Values of at least 25, or at least 30, or at least 35, when such absorbent structures 101 are formed to have an average basis weight of superabsorbent material 317 of 500 gsm and a combined basis weight of one or more adhesives of 7% of the basis weight of the superabsorbent material 317. As another example, the code DD53, representing an absorbent structure 101 formed to have an average basis weight of superabsorbent material 317 of 500 gsm and a combined basis weight of one or more adhesives of 3% of the basis weight of the superabsorbent material 317, had a Wet Pad Integrity value of 3, which higher than the Wet Pad Integrity value for the corresponding code NN53 (Wet Pad Integrity value for NN53 is 0) formed according to the Nordson process which could not withstand even a single shake from the Wet Pad Integrity Test Method.

In this manner, it can be seen that the processes 300 and 400 produce absorbent structures 101 having a superior Wet Pad Integrity than absorbent bodies produced by prior art processes. For example, the processes disclosed herein include gravity feeding superabsorbent material 317 forming a stream of superabsorbent toward a web material 303 and further include spraying both a first side and a second side of the stream with adhesive. As described herein, the adhesive intermixes with the superabsorbent material 317 prior to deposition onto the web material 303. Accordingly, based on the above results, these processes are additionally capable of producing absorbent structures 101 having Wet Pad Integrity values greater than or equal to 20, according to the Wet Pad Integrity Test, at least when used to produce structures 101 which have superabsorbent material 317 disposed in an amount equal to 500 gsm and adhesive disposed in an amount equal to 7% by weight, of the weight of the superabsorbent material 317. Of course, as described in more detail with respect to process 400, it could be the case that the process includes gravity feeding two separate streams of superabsorbent material 317 toward web material 303 and spraying adhesive at first and second sides of both streams of superabsorbent material 317. Further, such processes according to the present disclosure may be described as being able to form absorbent structures 101 having Wet Pad Integrity Values of at least 25, or at least 30, or at least 35, when such absorbent structures 101 are formed to have an average basis weight of superabsorbent material 317 of 500 gsm and a combined basis weight of one or more adhesives of 7% of the basis weight of the superabsorbent material 317. Additionally, such processes according to the present disclosure are able to form absorbent structures 101 having Wet Pad Integrity Values of at least 1, or at least 2, or at least 3, when such absorbent structures 101 are formed to have an average basis weight of superabsorbent material 317 of 500 gsm and a combined basis weight of one or more adhesives of 3% of the basis weight of the superabsorbent material 317

### Pad Uniformity Test Method Results

Another feature of the processes described herein, as compared to the Nordson process, is that the processes described herein are able to produce absorbent structures 101 which have a more uniform distribution of superabsorbent material 317 and adhesive fibers 316 throughout the formed structures 101 than absorbent bodies formed according to the Nordson process. This higher uniformity can allow the absorbent structures 101 to be thinner, more flexible, and handle fluid better than absorbent bodies having similar basis weights of superabsorbent material and adhesive.

In order to compare the distribution of superabsorbent material 317 and adhesive fibers 316, a number of different absorbent structures 101 were formed according to aspects of the present disclosure and compared to a number of different absorbent bodies formed according to the Nordson process. As can be seen in TABLES 3A-3E, absorbent structures 101 formed according to aspects of the present disclosure are labeled as codes DDD23, DDD24, DDD27, DDD33, DDD34, DDD44, DDD45, DDD56, DDD62, DDD66, and DDD67. These codes were formed by a process similar to that described above with respect to the Second Exemplary Absorbent Structures. The absorbent structures formed according to the Nordson process are labeled as NNN23, NNN24, NNN27, NNN33, NNN34, NNN44, NNN45, NNN56, NNN62, NNN66, and NNN67. These codes were formed by a process similar to that described above with respect to the Third Exemplary Absorbent Structures.

The TABLES 3A-3E reporting the results of the various codes according to the Pad Uniformity Test Method represent the results from a single sample for each code. The CD GL Var. column details the variance in the gray level of the sample over a portion of the sample extending the cross-direction, as determined according to the Pad Uniformity Test Method. Lower gray level variance values indicate a generally more uniform structure, since the variance in the determined gray levels is lower. The CD Mean GL column reports the determined mean gray level value for the sample, as determined according to the Pad Uniformity Test Method, while the GL % COV value reports the calculated gray level variability normalized with respect to the mean gray level. For example, the GL % COV value is determined by dividing the gray level standard deviation by the mean gray level, for a given sample, and multiplying such a calculated value by 100%. Determining all of these values is described in more detail below with respect to the Pad Uniformity Test Method.

TABLE 3A

| | SAM | % | CD GL | CD GL Var. | CD Mean | CD Mean GL | GL | GL % COV |
|---|---|---|---|---|---|---|---|---|
| Code | gsm | Adh | Var. | Std. Dev. | GL | Std. Dev. | % COV | Std. Dev. |
| DDD23 | 200 | 3 | 650.8 | 73.1 | 108.2 | 3.0 | 24.5 | 1.2 |
| NNN23 | 200 | 3 | 879.3 | 88.3 | 104.4 | 3.1 | 29.1 | 0.9 |
| DDD24 | 200 | 4 | 653.2 | 67.4 | 106.7 | 2.4 | 25.4 | 1.0 |
| NNN24 | 200 | 4 | 834.3 | 117.7 | 103.2 | 3.5 | 28.7 | 0.9 |
| DDD27 | 200 | 7 | 761.6 | 105.6 | 96.3 | 3.3 | 30.0 | 1.1 |
| NNN27 | 200 | 7 | 762.0 | 94.7 | 107.0 | 3.0 | 27.1 | 0.9 |

TABLE 3B

| | SAM | % | CD GL | CD GL Var. | CD Mean | CD Mean GL | GL | GL % COV |
|---|---|---|---|---|---|---|---|---|
| Code | gsm | Adh | Var. | Std. Dev. | GL | Std. Dev. | % COV | Std. Dev. |
| DDD33 | 300 | 3 | 577.7 | 77.5 | 88.8 | 2.5 | 28.8 | 1.7 |
| NNN33 | 300 | 3 | 677.4 | 100.4 | 98.3 | 3.3 | 27.8 | 1.5 |
| DDD34 | 300 | 4 | 623.5 | 76.9 | 86.0 | 2.4 | 30.7 | 1.5 |
| NNN34 | 300 | 4 | 716.0 | 111.4 | 88.5 | 3.1 | 31.6 | 1.5 |

TABLE 3C

| | SAM | % | CD GL | CD GL Var. | CD Mean | CD Mean GL | GL | GL % COV |
|---|---|---|---|---|---|---|---|---|
| Code | gsm | Adh | Var. | Std. Dev. | GL | Std. Dev. | % COV | Std. Dev. |
| DDD44 | 400 | 4 | 476.6 | 83.3 | 76.2 | 3.1 | 30.2 | 2.0 |
| NNN44 | 400 | 4 | 592.3 | 98.3 | 72.5 | 2.7 | 34.7 | 2.4 |
| DDD45 | 400 | 5 | 466.4 | 65.2 | 72.8 | 2.4 | 31.1 | 1.8 |
| NNN45 | 400 | 5 | 640.3 | 99.6 | 73.2 | 3.0 | 36.1 | 1.7 |

TABLE 3D

| | SAM | % | CD GL | CD GL Var. | CD Mean | CD Mean GL | GL | GL % COV |
|---|---|---|---|---|---|---|---|---|
| Code | gsm | Adh | Var. | Std. Dev. | GL | Std. Dev. | % COV | Std. Dev. |
| DDD56 | 500 | 6 | 400.0 | 47.3 | 63.6 | 3.0 | 33.4 | 1.9 |
| NNN56 | 500 | 6 | 513.0 | 101.1 | 61.9 | 3.4 | 38.0 | 2.6 |

TABLE 3E

| | SAM | % | CD GL | CD GL Var. | CD Mean | CD Mean GL | GL | GL % COV |
|---|---|---|---|---|---|---|---|---|
| Code | gsm | Adh | Var. | Std. Dev. | GL | Std. Dev. | % COV | Std. Dev. |
| DDD62 | 600 | 2 | 356.2 | 64.7 | 68.0 | 2.7 | 29.2 | 2.2 |
| NNN62 | 600 | 2 | 531.6 | 143.4 | 68.2 | 4.0 | 36.1 | 3.9 |
| DDD66 | 600 | 6 | 275.1 | 60.1 | 58.9 | 2.8 | 29.5 | 2.5 |
| NNN66 | 600 | 6 | 498.4 | 71.5 | 62.5 | 2.8 | 37.5 | 2.6 |
| DDD67 | 600 | 7 | 254.2 | 51.9 | 58.0 | 3.2 | 29.1 | 2.8 |
| NNN67 | 600 | 7 | 532.2 | 106.4 | 62.8 | 4.4 | 35.9 | 6.5 |

As can be seen in the TABLES 3A-3E, the structures 101 produced by the processes described herein result in much lower gray level variance than the absorbent bodies formed according to the Nordson process. For example, the codes DDD23 and DDD24 have CD GL Var. values of less than 815, less than 800, less than 750, or less than 700, as determined according to the Pad Uniformity Test Method. Such CD GL Var. values are all less than the CD GL Var. values of the corresponding NNN23 and NNN24 codes. Put another way, absorbent structures 101 formed according to aspects of the present disclosure which have superabsorbent material 317 disposed at a basis weight of 200 gsm and one or more adhesives disposed in a combined basis weight that is less than 4%, by weight, of the basis weight of superabsorbent material 317, may have CD GL Var. values of less than 815, less than 800, less than 750, or less than 700, as determined according to the Pad Uniformity Test Method. In some of these embodiments, the one or more adhesives may be disposed in a combined basis weight of between 3% and 4%, by weight, of the basis weight of superabsorbent material 317.

Further examples indicate that codes DDD33 and DDD34 have CD GL Var. values of less than 675, less than 650, or less than 625, as determined according to the Pad Uniformity Test Method. Such CD GL Var. values are all less than the CD GL Var. values of the corresponding NNN33 and NNN34 codes. Put another way, absorbent structures 101 formed according to aspects of the present disclosure which have superabsorbent material 317 disposed at a basis weight of 300 gsm and one or more adhesives disposed in a combined basis weight that is less than 4%, by weight, of the basis weight of superabsorbent material 317, may have CD GL Var. values of less than 675, less than 650, or less than 625, as determined according to the Pad Uniformity Test Method. In some of these embodiments, the one or more adhesives may be disposed in a combined basis weight of between 3% and 4%, by weight, of the basis weight of superabsorbent material 317.

Still further examples indicate that codes DDD44 and DDD45 have CD GL Var. values of less than 575, or less than 550, less than 525, or less than 500, as determined according to the Pad Uniformity Test Method. Such CD GL Var. values are all less than the CD GL Var. values of the corresponding NNN44 and NNN45 codes. Put another way, absorbent structures 101 formed according to aspects of the present disclosure which have superabsorbent material 317 disposed at a basis weight of 400 gsm and one or more adhesives disposed in a combined basis weight that is less than 5%, by weight, of the basis weight of superabsorbent material 317, may have CD GL Var. values of less than 585, less than 550, less than 525, or less than 500, as determined according to the Pad Uniformity Test Method. In some of these embodiments, the one or more adhesives may be disposed in a combined basis weight of between 4% and 5%, by weight, of the basis weight of superabsorbent material 317.

More examples indicate that code DDD56 has a CD GL Var. value of less than 500, or less than 475, less than 450, or less than 425, as determined according to the Pad Uniformity Test Method. Such CD GL Var. values are all less than the CD GL Var. value of the corresponding NNN56 code. Put another way, absorbent structures 101 formed according to aspects of the present disclosure which have superabsorbent material 317 disposed at a basis weight of 500 gsm and one or more adhesives disposed in a combined basis weight that is 6%, by weight, of the basis weight of superabsorbent material 317, may have CD GL Var. values of less than 500, less than 475, less than 450, or less than 425, as determined according to the Pad Uniformity Test Method.

The TABLE 3E highlights that especially at high basis weights of superabsorbent material 317, the absorbent structures 101 formed according to aspects of the present disclosure are superior to the absorbent structures formed according to the Nordson process. Codes DDD62, DDD63, and DDD67 have CD GL Var. values of less than 475, less than 450, less than 425, less than 400, or less than 375, as determined according to the Pad Uniformity Test Method. In particular, the codes DDD66 and DDD67 have CD GL Var. values of less than 350, less than 325, or less than 300. Such CD GL Var. values are all less than the CD GL Var. values of the corresponding NNN62, NNN66, and NNN67 codes. Put another way, absorbent structures 101 formed according to aspects of the present disclosure which have superabsorbent material 317 disposed at a basis weight of 600 gsm and one or more adhesives disposed in a combined basis weight that is less than 7%, by weight, of the basis weight of superabsorbent material 317, may have CD GL Var. values of less than 475, less than 450, less than 425, less than 400, or less than 375, as determined according to the Pad Uniformity Test Method. In some of these embodiments, the one or more adhesives may be disposed in a combined basis weight of between 2% and 7%, by weight, of the basis weight of superabsorbent material 317. In further of these examples, where the basis weights of the one or more adhesives are disposed range between 6% and 7%, by weight, of the basis weight of superabsorbent material 317, such absorbent structures 101 may have CD GL Var. values of less than 350, less than 325, or less than 300.

Additional characterizations of the absorbent structures 101 formed according to aspects of the present disclosure may include the following: absorbent structures 101 having a basis weight of superabsorbent material 317 of between 500 gsm and 600 gsm may have CD GL Var. values of less than 475, less than 450, or less than 425. In at least some of these embodiments, one or more adhesives present in such structures 101 may have a combined basis weight of less than 7%, or less than 6%, or between 6% and 7%, or between 2% and 7%. Absorbent structures 101 having a basis weight of superabsorbent material 317 of between 400 gsm and 500 gsm may have CD GL Var. values of less than 510, less than 500, less than 490, or less than 480. In at least some of these embodiments, one or more adhesives present in such structures 101 may have a combined basis weight of less than 6%, or less than 5%, or between 4% and 6%. Absorbent structures 101 having a basis weight of superabsorbent material 317 of between 300 gsm and 400 gsm may have CD GL Var. values of less than 590 or less than 580. In at least some of these embodiments, one or more adhesives present in such structures 101 may have a combined basis weight of less than 5%, or less than 4%, or between 3% and 5%. Absorbent structures 101 having a basis weight of superabsorbent material 317 of between 200 gsm and 300 gsm, and wherein one or more adhesives present in such structures 101 have a combined basis weight of between 3% and 4%, may have CD GL Var. values of less than 675, less than 665, or less than 655.

When using the GL % COV values, it can be seen that the absorbent structures 101 formed according to aspects of the present disclosure have generally lower variance in the determined gray level across different basis weights. For example, the codes DDD44, DDD45, DDD56, DDD62, DDD66, and DDD67 all have GL % COV values of less than 34.5, less than 34, or less than 33.5. Such GL % COV values are all less than the GL % COV values of the corresponding NNN44, NNN45, NNN56, NNN62, NNN66, and NNN67 codes. Put another way, absorbent structures 101 formed according to aspects of the present disclosure which have superabsorbent material 317 disposed at a basis weight of between 400 gsm and 600 gsm, and one or more adhesives disposed in a combined basis weight that is less than 7%, by weight, of the basis weight of superabsorbent material 317, may have GL % COV values of less than 34.5, less than 34, or less than 33.5, as determined according to the Pad Uniformity Test Method. In some of these embodiments, the one or more adhesives may be disposed in a combined basis weight of between 4% and 7%, or between 4% and 6%, by weight, of the basis weight of superabsorbent material 317. In further of any of these embodiments, the superabsorbent material 317 may be disposed at a basis weight of between 400 gsm and 500 gsm.

As another example, the codes DDD34, DDD44, and DDD45 all have GL % COV values of less than 31.5, or less than 31.3. The lowest GL % COV value of the corresponding NNN34, NNN44, and NNN45 codes is 31.6. Put another way, absorbent structures 101 formed according to aspects of the present disclosure which have superabsorbent material 317 disposed at a basis weight of between 300 gsm and 400 gsm, and one or more adhesives disposed in a combined basis weight that is between 4% and 5%, by weight, of the basis weight of superabsorbent particles 318, may have GL % COV values of less than 31.5, or less than 31.3, as determined according to the Pad Uniformity Test Method.

Absorbent Articles with High SAM Content and Low Void Volumes

It has been found that particular absorbent articles of the present disclosure, such as articles 10, 210, containing absorbent structures 101 having high superabsorbent material 317 content, by weight of absorbent material, where the superabsorbent material 317 has particular performance properties can produce surprising fluid intake results. It is typically desired in absorbent articles to provide for a fast fluid intake rate, and in particular a fast first cradle intake rate, to ensure the articles draw liquids away from a wearer's skin within a sufficiently quick time period to maintain skin dryness and prevent skin irritation. Use of absorbent materials such as pulp fluff and superabsorbent material provide such absorbent articles with beneficial fluid intake speeds to effect such a fast fluid intake. Absorbent articles are presently designed with sufficient dry void volumes to act as temporary liquid reservoirs where fluid may collect prior to being absorbed by the absorbent material of the absorbent structure or other portions of the article. Such dry void volumes help to provide a fast fluid intake time by allowing space for fluid to flow to within the article and away from the skin. It has previously been thought that high dry void volumes would be required to effect fast fluid intake speeds even where the superabsorbent materials utilized had fast liquid uptake rate kinetics.

Utilizing high superabsorbent content absorbent bodies, for example similar to any of absorbent structures 101 described herein, it has been found that absorbent articles 10, 210 having high overall superabsorbent material 317 content, by weight of overall article absorbent material, have surprisingly beneficial first cradle intake rates at dry void volumes substantially lower than found in current products. More specifically, it has been found that absorbent articles 10, 210 according the present disclosure are able to achieve surprising first cradle intake rates, according to the Cradle Intake Test Method described herein, while having very low dry void volumes. While there is not an exact correlation between dry void volume and article thinness, low dry void volumes tend to be associated within thinner materials. Accordingly, the absorbent articles 10, 210 of the present disclosure are able to achieve desirable first cradle intake times with surprisingly low dry void volumes thereby allowing such articles to be thin, providing superior fit and comfort to a wearer.

Key features of such absorbent articles 10, 210 include where the absorbent articles have a high superabsorbent material 317 content, by weight of absorbent material, such as greater than about 80% superabsorbent material, greater than about 85% superabsorbent material, greater than about 90% superabsorbent material, greater than about 95% superabsorbent material, or having 100% superabsorbent material (e.g. pulp-free, or fluff-less), by weight of absorbent material. Such articles 10, 210 may achieve these percentages where absorbent layers within absorbent structures 101 of the articles 10, 210 have high superabsorbent material 317 content and where other portions of the articles 10, 210 lack other absorbent material—such as cellulose based absorbent material. Alternatively, absorbent layer(s) of the absorbent structures 101 of such absorbent articles 10, 210 may comprise 100% superabsorbent material, by weight of absorbent material, and other portions of such articles 10, 210 may have relatively low amounts of cellulose-based absorbent material.

Another key feature is that the absorbent articles 10, 210 comprise an article dry void volume of less than 0.45 cm³/cm², as determined by the Dry Void Volume Determination Test Method described herein. Although, sufficient first cradle intake times have been achieved with articles 10, 210 of the present disclosure having dry void volumes of less than about 0.40 cm³/cm², less than about 0.35 cm³/cm², less than about 0.30 cm³/cm², less than about 0.25 cm³/cm², and even less than about 0.20 cm³/cm².

A further key feature in enabling sufficient first cradle intake times while having low dry void volume is utilizing superabsorbent materials 317 having low vortex times. Vortex times are one measurement of an uptake rate of liquid of superabsorbent materials. Vortex times of this disclosure are calculated according to the Vortex Time Test Method described herein. When utilized in absorbent articles, such as articles 10, 210, having dry void volumes of about 0.45 cm³/cm² or less, it has been found to be beneficial for the superabsorbent materials 317 to have vortex times of less than about 40 s, or less than about 37 s, or less than about 35 s, or less than about 30 s, or less than about 27 s, or less than about 25 s, or less than about 20 s, or less than about 14 s.

Although a primary driver of fast first cradle intake times may be a vortex time of the superabsorbent material 317 used in such low dry void volume absorbent articles, further properties of the superabsorbent materials 317 are critical to good fluid intake performance of the absorbent articles 10, 210 of the present disclosure as well as performance in other areas—such as leakage rate and overall fluid capacity. Accordingly, while having low vortex times, useful superabsorbent materials 317 in low dry void volume articles 10, 210 having fast first cradle intake times should also have centrifuge retention capacity (CRC) values of between about 20 g/g and about 45 g/g, or between about 28 g/g and about 36 g/g. The CRC values of this disclosure are calculated according to the CRC Test Method detailed herein. As well, the superabsorbent materials 317 should have free-swell gel bed permeability (GBP) values of between 8 and 150 μm², or between about 20 and 100 μm², or between about 30 and 80 μm². The GBP values of this disclosure are calculated according to the Free-Swell Gel Bed Permeability Test Method (GBP Test Method) described herein. It may be further desirable for the superabsorbent materials 317 to have an absorbency under load (AUL) value of between 9 g/g and 20 g/g. The AUL values of this disclosure are calculated according to the Absorbency Under Load (AUL) Test Method detailed herein.

Various absorbent articles of the present disclosure 10, 210 having low dry void volumes, as will be described in more detail below, have been surprisingly able to achieve first cradle intake times of less than 25 s, according to the Cradle Intake Time Test Method. Other absorbent articles of the present disclosure 10, 210 have been able to achieve first cradle intake times of less than 22 s, less than 20 s, and even less than 15 s. These times compare favorably to commercially available comparative absorbent articles which have higher dry void volumes and/or superabsorbent materials with insufficient performance properties to produce such fast first cradle intake times at low dry void volumes.

### Exemplary Low Dry Void Volume Absorbent Articles

Absorbent articles 10, 210 according to the present disclosure were formed to include absorbent structures 101 formed according to process 400. Such absorbent articles are listed as codes Example 1 through Example 22 (collectively “Example Codes”). These Example Codes 1-22 were formed with absorbent structures according to process 400 with a set-up similar to that described with respect to the Second Exemplary Absorbent Structures described previously and comprising 460 gsm of superabsorbent material 317, with 230 gsm of superabsorbent material applied at each of the deposition stations 302a, 302b. The superabsorbent material of the Example Codes 1-22 was stabilized with adhesive applied at an add-on rate of 5%, split evenly between adhesive applicators 307, 309, 333, and 335 of deposition stations 302a, 302b. The superabsorbent material at each deposition station 302a, 302b, was applied at the same width to form a relatively uniform absorbent layer in both the machine and cross-machine directions 330, 338. Such absorbent layers as described below may be considered to include portions of the absorbent structure 101 or body 34 which include absorbent material and any stabilizing component (e.g. adhesive—for example, superabsorbent material 317 and adhesives 308, 310, 334, and/or 336). For example, with respect to absorbent structures 101, the absorbent layer would be considered to comprise the absorbent mixture 320. Such absorbent structures 101 formed by the process 400 were then incorporated into a standard absorbent article having dimensions typical for a commercially available size 4 Huggies Little Snuggler® diaper absorbent article, with the absorbent structures 101 forming the complete absorbent bodies of the diaper absorbent articles. For example, the formed absorbent structures 101 were positioned between a liquid permeable bodyside liner and a liquid impermeable outer cover and included other components such as fastening systems and containment flap systems which are immaterial to the Cradle Intake Test Method. Further details of the critical components of the absorbent articles of the Example Codes 1-22 are described below with respect to the individual Example Codes 1-22.

TABLE 4

| | | Acquisition and/or | | | |
|---|---|---|---|---|---|
| | Bodyside liner | distribution | Top corewrap | Bottom corewrap | Superabsorbent |
| Code | material (gsm) | layer material (gsm) | material (gsm) | material (gsm) | Material |
| Example 1 | 12 gsm dusted | None | 12 gsm dSB | 12 gsm dSB | Example SAM 1 |
| | spunbond | | | | |
| | (dSB) | | | | |
| Example 2 | 12 gsm dSB | None | 20 gsm BCW | 20 gsm BCW | Example SAM 1 |
| Example 3 | 12 gsm dSB | 35 gsm TABCW | 12 gsm dSB | 12 gsm dSB | Example SAM 1 |
| Example 4 | 12 gsm dSB | 55 gsm TABCW | 20 gsm BCW | 20 gsm BCW | Example SAM 1 |
| Example 5 | 12 gsm dSB | 100 gsm TABCW | 20 gsm BCW | 20 gsm BCW | Example SAM 1 |
| Example 6 | 12 gsm dSB | 55 gsm TABCW | 60 gsm coform | 60 gsm coform | Example SAM 1 |
| Example 7 | 12 gsm dSB | 55 gsm TABCW | 55 gsm TABCW | 55 gsm TABCW | Example SAM 1 |
| Example 8 | 12 gsm dSB | 100 gsm TABCW | 55 gsm TABCW | 55 gsm TABCW | Example SAM 1 |
| Example 9 | 12 gsm dSB | None | 20 gsm BCW | 20 gsm BCW | Example SAM 2 |
| Example 10 | 12 gsm dSB | 35 gsm TABCW | 20 gsm BCW | 20 gsm BCW | Example SAM 2 |
| Example 11 | 12 gsm dSB | 55 gsm TABCW | 20 gsm BCW | 20 gsm BCW | Example SAM 2 |
| Example 12 | 12 gsm dSB | 100 gsm TABCW | 12 gsm dSB | 12 gsm dSB | Example SAM 2 |
| Example 13 | 12 gsm dSB | 55 gsm TABCW | 55 gsm TABCW | 55 gsm TABCW | Example SAM 2 |
| Example 14 | 12 gsm dSB | 55 gsm TABCW | 20 gsm BCW | 20 gsm BCW | Example SAM 3 |
| Example 15 | 12 gsm dSB | 100 gsm TABCW | 12 gsm dSB | 12 gsm dSB | Example SAM 4 |
| Example 16 | 12 gsm dSB | None | 12 gsm dSB | 12 gsm dSB | Example SAM 5 |
| Example 17 | 12 gsm dSB | 35 gsm TABCW | 20 gsm BCW | 20 gsm BCW | Example SAM 5 |
| Example 18 | 12 gsm dSB | 55 gsm TABCW | 20 gsm BCW | 20 gsm BCW | Example SAM 5 |
| Example 19 | 12 gsm dSB | 100 gsm TABCW | 20 gsm BCW | 20 gsm BCW | Example SAM 5 |
| Example 20 | 12 gsm dSB | 100 gsm TABCW | 60 gsm coform | 60 gsm coform | Example SAM 5 |
| Example 21 | 12 gsm dSB | 35 gsm TABCW | 55 gsm TABCW | 55 gsm TABCW | Example SAM 5 |
| Example 22 | 12 gsm dSB | 100 gsm TABCW | 55 gsm TABCW | 55 gsm TABCW | Example SAM 5 |
| Comparative | 12 gsm dSB | None | 20 gsm BCW | 20 gsm BCW | Comparative SAM 1 |
| Example 1 | | | | | |
| Comparative | 12 gsm dSB | 35 gsm TABCW | 12 gsm dSB | 12 gsm dSB | Comparative SAM 1 |
| Example 2 | | | | | |
| Comparative | 12 gsm dSB | 35 gsm BCW | 20 gsm BCW | 20 gsm BCW | Comparative SAM 1 |
| Example 3 | | | | | |
| Comparative | 12 gsm dSB | 100 gsm TABCW | 12 gsm dSB | 12 gsm dSB | Comparative SAM 1 |
| Example 4 | | | | | |
| Comparative | 12 gsm dSB | 35 gsm TABCW | 60 gsm coform | 60 gsm coform | Comparative SAM 1 |
| Example 5 | | | | | |
| Comparative | 12 gsm dSB | 35 gsm TABCW | 55 gsm TABCW | 55 gsm TABCW | Comparative SAM 1 |
| Example 6 | | | | | |
| Comparative | 12 gsm dSB | 100 gsm TABCW | 55 gsm TABCW | 55 gsm TABCW | Comparative SAM 1 |
| Example 7 | | | | | |

### Comparative Absorbent Articles

Additional codes, labeled as Comparative Examples 1-7, were formed with absorbent bodies formed in the same manner as the Example Codes. Although, the Comparative Examples 1-7 were formed with a comparative superabsorbent material, labeled as Comparative SAM 1, having a high vortex time (63 s) relative to the superabsorbent materials used to form the Example Codes.

A first commercially available comparative example, labeled herein as Comparative Example 8, containing a high superabsorbent material content absorbent body is the Pampers Swaddlers® Active Baby size 4 absorbent article, which was obtained commercially. These absorbent articles have a bodyside liner formed of a spunmelt material having a basis weight of approximately 35 gsm. The acquisition/distribution layer comprises an open nonwoven structure material having a basis weight of approximately 52 gsm positioned proximate the bodyside liner and a layer of stiffened cellulose fiber having a basis weight of approximately 133 gsm. The absorbent body/structure comprises approximately 100% superabsorbent material by weight bound with adhesive and wrapped in spunmelt nonwoven materials. The combination of the superabsorbent material, the adhesive, and the spunmelt materials have a basis weight of approximately 374 gsm. When compared to the total absorbent material of the Comparative Example 8 absorbent article, the superabsorbent material comprises approximately 74% by weight of total absorbent material of the Comparative Example 8 article (e.g. including the 133 gsm of stiffened cellulose fiber used as the acquisition/distribution layer). It is believed that the structure of this commercially available product is similar to that described with respect to U.S. Pat. No. 7,851,667 to Becker et al.

A second commercially available comparative example, labeled herein as Comparative Example 9, containing a high superabsorbent material content absorbent body is Huggies® Ultimate Nappy Pants size 4. These absorbent articles have a bodyside liner formed of a BCW material having a basis weight of approximately 26 gsm. The absorbent body/structure comprises approximately 100% superabsorbent material by weight of absorbent material and present in an amount of about 320 gsm. These absorbent articles have an additional material internal to the absorbent body to provide additional void volume to the absorbent article. This internal material is located between a first absorbent layer and a second absorbent layer. Each of the absorbent layers were formed of superabsorbent material having a basis weight of approximately 160 gsm. This internal material may be considered an internal acquisition/distribution layer and comprises a BCW material having a basis weight of approximately 40 gsm. Additionally, the top corewrap material is a spunlace material having basis weight of approximately 40 gsm and the bottom corewrap material is a spunlace material having basis weights of approximately 26 gsm. The superabsorbent material has a vortex time of 41 s, and is referred herein as Comparative SAM 2.

As described above, it was surprising that the Example Codes 1-22 having low dry void volumes were able to perform well according to the Cradle Intake Test Method, indicating a surprising ability of such Example Codes 1-22 to have a low leakage rate and to sufficiently maintain skin dryness of a wearer upon liquid insult. Key attributes of the Example and Comparative codes and their results according to the Cradle Intake Test Method are shown below.

With respect to Table 5, the Dry Void Volume Above Absorbent Layer parameter includes the void volume of the top corewrap material and all materials above the top corewrap material, including the bodyside liner material. The Dry Void Volume Below Absorbent Layer parameter includes the void volume of the bottom corewrap material and all materials between the bottom corewrap material and the outer cover material.

TABLE 5

| | | | | | Dry Void Volume |
|---|---|---|---|---|---|
| | | | | | of Absorbent |
| | | | | | Structure (less |
| | | Article Dry Void | Dry Void Volume | Dry Void Volume | corewrap |
| | Superabsorbent | Volume | Above Absorbent | Below Absorbent | materials) |
| Code | Vortex Time (s) | (cm³/cm²) | Layer (cm³/cm²) | Layer (cm³/cm²) | (cm³/cm²) |
| Example 1 | 29 | 0.147 | 0.024 | 0.012 | 0.111 |
| Example 2 | 29 | 0.171 | 0.036 | 0.024 | 0.111 |
| Example 3 | 29 | 0.221 | 0.098 | 0.012 | 0.111 |
| Example 4 | 29 | 0.300 | 0.165 | 0.024 | 0.111 |
| Example 5 | 29 | 0.346 | 0.211 | 0.024 | 0.111 |
| Example 6 | 29 | 0.404 | 0.217 | 0.076 | 0.111 |
| Example 7 | 29 | 0.510 | 0.270 | 0.129 | 0.111 |
| Example 8 | 29 | 0.556 | 0.316 | 0.129 | 0.111 |
| Example 9 | 14 | 0.171 | 0.036 | 0.024 | 0.111 |
| Example 10 | 14 | 0.245 | 0.110 | 0.024 | 0.111 |
| Example 11 | 14 | 0.300 | 0.165 | 0.024 | 0.111 |
| Example 12 | 14 | 0.322 | 0.199 | 0.012 | 0.111 |
| Example 13 | 14 | 0.510 | 0.270 | 0.129 | 0.111 |
| Example 14 | 18 | 0.300 | 0.165 | 0.024 | 0.111 |
| Example 15 | 38 | 0.322 | 0.199 | 0.012 | 0.111 |
| Example 16 | 41 | 0.147 | 0.024 | 0.012 | 0.111 |
| Example 17 | 41 | 0.245 | 0.110 | 0.024 | 0.111 |
| Example 18 | 41 | 0.300 | 0.165 | 0.024 | 0.111 |
| Example 19 | 41 | 0.346 | 0.211 | 0.024 | 0.111 |
| Example 20 | 41 | 0.450 | 0.263 | 0.076 | 0.111 |
| Example 21 | 41 | 0.455 | 0.215 | 0.129 | 0.111 |
| Example 22 | 41 | 0.556 | 0.316 | 0.129 | 0.111 |
| Comparative | 63 | 0.171 | 0.036 | 0.024 | 0.111 |
| Example 1 | | | | | |
| Comparative | 63 | 0.221 | 0.098 | 0.012 | 0.111 |
| Example 2 | | | | | |
| Comparative | 63 | 0.245 | 0.11 | 0.024 | 0.111 |
| Example 3 | | | | | |
| Comparative | 63 | 0.322 | 0.199 | 0.012 | 0.111 |
| Example 4 | | | | | |
| Comparative | 63 | 0.349 | 0.162 | 0.076 | 0.111 |
| Example 5 | | | | | |
| Comparative | 63 | 0.455 | 0.215 | 0.129 | 0.111 |
| Example 6 | | | | | |
| Comparative | 63 | 0.556 | 0.316 | 0.129 | 0.111 |
| Example 7 | | | | | |
| Comparative | undetermined | 0.523 | 0.386 | 0.012 | 0.125 |
| Example 8 | | | | | |
| Comparative | 41 | 0.280 | 0.059 | 0.020 | 0.197 |
| Example 9 | | | | | |

TABLE 6

| | | | Superabsorbent Gel | | |
|---|---|---|---|---|---|
| | Superabsorbent | Superabsorbent | Bed Permeability | Article Dry Void | Cradle Intake |
| Code | Vortex Time (s) | CRC Value (g/g) | (μm²) | Volume (cm³/cm²) | Time (s) |
| Example 1 | 29 | 29.6 | 39.2 | 0.147 | 22.3 |
| Example 2 | 29 | 29.6 | 39.2 | 0.171 | 18.5 |
| Example 3 | 29 | 29.6 | 39.2 | 0.221 | 22.6 |
| Example 4 | 29 | 29.6 | 39.2 | 0.300 | 15.1 |
| Example 5 | 29 | 29.6 | 39.2 | 0.346 | 13.4 |
| Example 6 | 29 | 29.6 | 39.2 | 0.404 | 12.6 |
| Example 7 | 29 | 29.6 | 39.2 | 0.510 | 11.2 |
| Example 8 | 29 | 29.6 | 39.2 | 0.556 | 10.0 |
| Example 9 | 14 | 25.8 | 71.3 | 0.171 | 13.2 |
| Example 10 | 14 | 25.8 | 71.3 | 0.245 | 11.3 |
| Example 11 | 14 | 25.8 | 71.3 | 0.300 | 11.0 |
| Example 12 | 14 | 25.8 | 71.3 | 0.322 | 12.1 |
| Example 13 | 14 | 25.8 | 71.3 | 0.510 | 7.8 |
| Example 14 | 18 | 28.6 | 12.6 | 0.300 | 13.3 |
| Example 15 | 38 | 32.1 | 29.9 | 0.322 | 25.1 |
| Example 16 | 41 | 29.3 | 48.2 | 0.147 | 32.8 |
| Example 17 | 41 | 29.3 | 48.2 | 0.245 | 24.3 |
| Example 18 | 41 | 29.3 | 48.2 | 0.300 | 22.8 |
| Example 19 | 41 | 29.3 | 48.2 | 0.346 | 17.4 |
| Example 20 | 41 | 29.3 | 48.2 | 0.450 | 13.3 |
| Example 21 | 41 | 29.3 | 48.2 | 0.455 | 14.6 |
| Example 22 | 41 | 29.3 | 48.2 | 0.556 | 13.8 |
| Comparative | 63 | 29.3 | 38.6 | 0.171 | 24.5 |
| Example 1 | | | | | |
| Comparative | 63 | 29.3 | 38.6 | 0.221 | 35.9 |
| Example 2 | | | | | |
| Comparative | 63 | 29.3 | 38.6 | 0.245 | 19.7 |
| Example 3 | | | | | |
| Comparative | 63 | 29.6 | 39.2 | 0.322 | 29.6 |
| Example 4 | | | | | |
| Comparative | 63 | 29.6 | 39.2 | 0.349 | 24.6 |
| Example 5 | | | | | |
| Comparative | 63 | 29.6 | 39.2 | 0.455 | 18.9 |
| Example 6 | | | | | |
| Comparative | 63 | 29.6 | 39.2 | 0.556 | 15.5 |
| Example 7 | | | | | |
| Comparative | undetermined | undetermined | undetermined | 0.523 | 9.6 |
| Example 8 | | | | | |
| Comparative | 41 | 31 | 39 | 0.280 | 21 |
| Example 9 | | | | | |

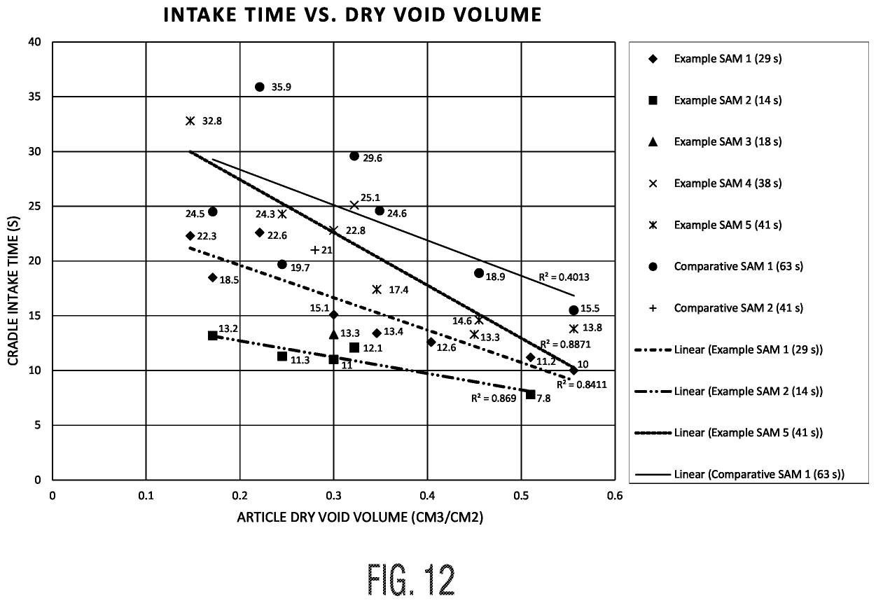

Key take-aways from TABLE 6 are that various of the Example Codes 1-22 were able to achieve desirable first cradle intake times of less than or equal to about 25 s at dry void volumes less than or equal to about 0.45 cm³/cm². The distribution of the Example Codes 1-22 representing Example SAMS 1-5 as well as Comparative Examples representing Comparative SAM 1 and Comparative SAM 2 are shown in graphical form in FIG. 12.

As can be seen in TABLE 6 and FIG. 12, at least Examples Codes 1-3, 9, 10, and 17 were all able to achieve first cradle intake times of less than or equal to 25 s while having dry void volumes of less than or equal to 0.25 cm³/cm². These Example Codes all had superabsorbent material with vortex times of less than or equal to 41 s. The Example Codes 4-8, 11-15, and 18-22, as well as Comparative Examples 8 and 9, while achieving a first cradle intake time of less than or equal to 25 s, all had dry void volumes of greater than 0.25 cm³/cm². The Example Code 16, while having a dry void volume of less than or equal to 0.25 cm³/cm² as well as a superabsorbent material having a vortex time less than or equal to 41 s, had first cradle intake times of greater than 25 s. Accordingly, absorbent articles 10, 210 of the present disclosure may prove beneficial in terms of first cradle intake rates as well as in fit and comfort where the absorbent articles 10, 210 have high superabsorbent material content along with dry void volumes of less than or equal to 0.25 cm³/cm², where the superabsorbent material has vortex times of less than or equal to 41 s. Although, it may be more preferable for the absorbent articles 10, 210 of the present disclosure to have superabsorbent material with vortex times of less than or equal to 29 s where the dry void volume is less than or equal to 0.25 cm³/cm². The Example Codes with superabsorbent materials having vortex times of less than or equal to 29 s were able to consistently achieve a first cradle intake time of less than 25 s at dry void volumes as low as 0.147 cm³/cm², a performance unable to be achieved by the Example Codes with superabsorbent materials having vortex times faster than 29 s.

A further subset of the Example Codes achieved even better results in that they were able to achieve first cradle intake times of less than or equal to 25 s at dry void volumes lower than or equal to 0.20 cm³/cm². For instance, Example Codes 1, 2, and 9 were all able to achieve first cradle intake times of less than or equal to 25 s while having dry void volumes of less than or equal to 0.20 cm³/cm². These particular Example Codes 1, 2, and 9 required superabsorbent materials with vortex times of less than or equal to 29 s to achieve first cradle intake times of less than or equal to 25 s. The Example Codes 3-8, 10-14, and 17-22, as well as Comparative Examples 8 and 9, while achieving a first cradle intake time of less than or equal to 25 s, all had dry void volumes of greater than 0.20 cm³/cm². The Example Code 16, while having a dry void volume of less than or equal to 0.20 cm³/cm² as well as a superabsorbent material having a vortex time of less than or equal to 41 s, had a first cradle intake times of greater than 25 s. Example Code 15 did not have a dry void volume of less than or equal to 0.20 cm³/cm² nor achieved a first cradle intake times of greater than 25 s. Accordingly, absorbent articles 10, 210 of the present disclosure may prove beneficial in terms of first cradle intake rates as well as in fit and comfort where the absorbent articles 10, 210 have high superabsorbent material content along with dry void volumes of less than or equal to 0.20 cm³/cm², where the superabsorbent material has vortex times of less than or equal to 29 s.

Reading further into the data in TABLE 6 and FIG. 12, it may be particularly beneficial for the absorbent articles 10, 210 of the present disclosure to have high superabsorbent material content along with dry void volumes of less than or equal to 0.45 cm³/cm² where the vortex time of the superabsorbent material is less than or equal to 29 s. In such embodiments, a broad range of dry void volumes may be utilized while still achieving a first cradle intake time of less than or equal to 25 s. For example, each of the Example Codes having superabsorbent materials with vortex times less than or equal to 29 s were all able to achieve a first cradle intake time of less than 25 s across a broad range of dry void volumes—between 0.147 cm³/cm² and 0.404 cm³/cm². Although Examples Code 17 and Comparative Code 9 were able to achieve a first cradle intake time of less than 25 s with superabsorbent materials having vortex times of 41 s and at dry void volumes lower than 0.45 cm³/cm², the first cradle intake times are approaching the 25 s limit. As can be seen, Example Code 16 fails to achieve the threshold of a first cradle intake time of 25 s at a dry void volume lower than those of Examples Code 17 and Comparative Code 9, indicating that a vortex time of 41 s is insufficient to provide for a first cradle intake time of less than 25 s across a dry void volume range of between 0.147 cm³/cm² and 0.404 cm³/cm². In more specific embodiments, it may be particularly beneficial for the absorbent articles 10, 210 of the present disclosure to have high superabsorbent material content along with dry void volumes of less than or equal to 0.404 cm³/cm² where the vortex time of the superabsorbent material is less than or equal to 29 s.

In a further aspect, various of the Example Codes were able to achieve first cradle intake times of less than or equal to 20 s. As seen in TABLE 6 and FIG. 12, the Example Codes 2, 4-6, 9-12, 14, 19, and 20 were able to achieve a first cradle intake time of less than or equal to 20 s with dry void volumes of less than or equal to 0.45 cm³/cm² where the superabsorbent materials have vortex times of less than or equal to 41 s. Although, in particular, Example Codes 2-6 were able to mostly achieve a first cradle intake time of less than or equal to 20 s across a broad range of dry void volumes—between 0.171 cm³/cm² and 0.404 cm³/cm². These Example Codes 2-6 had superabsorbent material having a vortex time of 29 s. Additionally, Example Codes 9-12 were all able to achieve a first cradle intake time of less than or equal to 20 s across a range of dry void volumes of between 0.171 cm³/cm² and 0.322 cm³/cm². These Example Codes 9-12 had superabsorbent material having a vortex time of 14 s. Although representing only a single data point with respect to superabsorbent material having a vortex time of 18 s, Example 14, having a dry void volume of 0.30 cm³/cm², was also able to achieve a first cradle intake time of less than 20 s. Conversely, although Example Codes 19 and 20 were able to achieve a first cradle intake time of less than or equal to 20 s while utilizing a superabsorbent material having a vortex time of 41 s, they required dry void volumes of between 0.346 cm³/cm² and 0.45 cm³/cm² to achieve this result. The rest of the Example Codes having superabsorbent material with a vortex time of 41 s and dry void volumes lower than 0.346 cm³/cm² were unable to achieve a first cradle intake time of less than or equal to 20 s. In more specific embodiments, it may be particularly beneficial for the absorbent articles 10, 210 of the present disclosure to have high superabsorbent material content along with dry void volumes of less than or equal to 0.404 cm³/cm² where the vortex time of the superabsorbent material is less than or equal to 29 s.

Accordingly, it may be desirable for the absorbent articles of the present disclosure to have high superabsorbent material content along with dry void volumes of between 0.171 cm³/cm² and 0.404 cm³/cm² where the superabsorbent material has a vortex time of less than or equal to 29 s. Alternatively, it may be desirable for the absorbent articles of the present disclosure to have high superabsorbent material content along with dry void volumes of between 0.171 cm³/cm² and 0.346 cm³/cm² where the superabsorbent material has a vortex time of less than or equal to 29 s. These combination of features allows such absorbent articles 10, 210 of the present disclosure to achieve a first cradle intake times of less than or equal to 20 s.

In still further aspects, certain of the Example Codes were able to achieve first cradle intake times of less than or equal to 15 s. As seen in TABLE 6 and FIG. 12, the Example Codes 5, 6, 9-12, 14, and 20 were able to achieve a first cradle intake time of less than or equal to 15 s with dry void volumes of less than or equal to 0.45 cm³/cm² with superabsorbent material having vortex times of less than or equal to 41 s. Although, in particular, Example Codes 5 and 6 were able to achieve a first cradle intake time of less than or equal to 15 s across a range of dry void volumes between 0.346 cm³/cm² and 0.404 cm³/cm². These Example Codes 5 and 6 had superabsorbent material having a vortex time of 29 s. Additionally, Example Codes 9-12 were all able to achieve a first cradle intake time of less than or equal to 15 s across a range of dry void volumes of between 0.171 cm³/cm² and 0.322 cm³/cm². These Example Codes 9-12 had superabsorbent material with a vortex time of 14 s. Although representing only a single data point with respect to superabsorbent material having a vortex time of 18 s, Example 14, having a dry void volume of 0.30 cm³/cm², was also able to achieve a first cradle intake time of less than 15 s. Conversely, although Example Code 20 was able to achieve a first cradle intake time of less than or equal to 15 s while utilizing a superabsorbent material having a vortex time of 41 s, Example Code 20 required a dry void volume of 0.45 cm³/cm² to achieve this result. The rest of the Example Codes having superabsorbent material with a vortex time of 41 s and dry void volumes lower than 0.45 cm³/cm² were unable to achieve a first cradle intake time of less than or equal to 15 s.

Accordingly, it may be desirable for absorbent articles 10, 210 of the present disclosure to have high superabsorbent material content along with dry void volumes of between 0.346 cm³/cm² and 0.404 cm³/cm² where the superabsorbent material has a vortex time of less than or equal to 29 s. It may also be desirable for absorbent articles 10, 210 of the present disclosure to have high superabsorbent material content along with dry void volumes of between 0.171 cm³/cm² and 0.404 cm³/cm² where the superabsorbent material has a vortex time of less than or equal to 18 s, or less than or equal to 14 s. These combinations of features allow such absorbent articles 10, 210 of the present disclosure to achieve a first cradle intake times of less than or equal to 15 s.

Another feature explored related to such low void volume articles, is the amount of void volume generation of the absorbent material during liquid uptake. For example, the absorbent portion of the structures 101, formed of superabsorbent material 317, swells when insulted with liquid. This swelling increases a height or thickness dimension of the absorbent layer(s) of the structures 101, thereby increasing the void volume of the absorbent layer(s). It was initially believed that the CRC value of the superabsorbent materials would be a primary driver of void volume generation—that the greater the CRC (a measure of absorbent capacity) of a superabsorbent material, the greater the void volume generated at partial saturation. However, after performing a test to determine a dependency between CRC and void volume generation, it was found that there was no appreciable correlation between CRC and void volume generation at partial saturation.

A further test was performed to determine whether superabsorbent material vortex time correlated with amount of void volume generation. TABLE 7 represents results of a Percent Void Volume Increase Test Method, described herein, showing how different of the Example Codes and Comparative Example codes performed according to the Percent Void Volume Increase Test Method at 20% saturation and 40% saturation of the sample. Since the void volume of the non-absorbent materials of the absorbent article is not dependent on fluid saturation, the percentage increase in void volume of the absorbent layer(s) (e.g. the absorbent materials 317 of the structure 101) may be determined by 1) subtracting the dry void volume of the absorbent layer(s) from the article or absorbent structure void volume, as determined according to the Percent Void Volume Increase Test Method, to determine the web void volume (e.g. the void volume of the web materials of the tested article or structure); 2) determining the article or structure void volume at 20%/40% saturation, according to the Percent Void Volume Increase Test Method; and 3) subtracting the web void volume from the determined article or structure void volume at 20%/40% saturation—yielding the increase in void volume of the absorbent layer(s) which may be readily translated to a percentage increase in void volume of the absorbent layer(s).

The results of the experiment indicate that a sufficient rate of void volume generation of the absorbent layer(s) is desired to quickly increase the effective void volume of the absorbent article or structure upon insult to help produce beneficial first cradle intake times. A fast rate of void volume generation would provide additional space for the fluid to collect within the article (e.g. away from the bodyside liner and a wearer's skin) while the liquid is being absorbed by the absorbent material as well as distributing throughout the absorbent structure. Additionally, the results indicate surprisingly that superabsorbent material vortex time has a strong correlation to void volume generation. However, it does not appear to be the case that the link between superabsorbent material vortex time and void volume generation is perfect. Accordingly, to achieve desired first cradle intake times in the articles 10, 210 of the present disclosure, superabsorbent materials with low vortex times should be selected. However, of these low vortex time superabsorbent materials, it may be preferred to select a subset of these superabsorbent materials which have the void volume generation properties as described below.

As can be seen from TABLE 7, it may be particularly beneficial to utilize a superabsorbent material which is able to provide an increase in void volume of the absorbent layer or layers of at least 180% at 20% saturation, according to the Percent Void Volume Increase Test Method. For instance, Example Codes 4, 6, 12, and 19 all had void volume increases of the absorbent layer (all of these codes had a single absorbent layer within the structures 101) of greater than or equal to 180% at 20% saturation, allowing them to achieve first cradle intake times of less than 20 s. All of these codes were able to achieve a first cradle intake time of less than 20 s at a void volume of less than 0.402 cm³/cm². Even Comparative Example 3, which had a relatively slower superabsorbent material vortex time (63 s vs. less than or equal to 29 s for the Example Codes), was able to achieve a first cradle intake time of less than 20 s by utilizing a superabsorbent material that was able to increase the void volume of the absorbent layer by at least 180%. Conversely, Example Codes 3 and Comparative Codes 2 and 4 had void volume increases of the absorbent layer of less than 180% and failed to achieve a first cradle intake of less than 20 s, where the dry void volume of these Codes were less than or equal to 0.402 cm³/cm². Although Comparative Example 5 was able to achieve a first cradle intake time of less than 20 s with the void volume of the absorbent layer only increasing by about 60%, Comparative Example 5 required a void volume of close to 0.45 cm³/cm² to achieve this result. These results may indicate that for absorbent articles 10, 210 of the present disclosure which have dry void volumes of less than about 0.402 cm³/cm², a void volume increase of greater than or equal to 180% at 20% saturation is desired to help ensure a fast first cradle intake time—such as less than 20 s. In further embodiments, it may be preferable to utilize a superabsorbent material which is able to provide an increase in void volume of the absorbent layer or layers of at least 196% at 20% saturation—particularly where the superabsorbent materials have vortex times of less than or equal to 41 s.

In another aspect, it may be desirable, where the dry void volume of the absorbent articles 10, 210 of the present disclosure are low—such as less than about 0.423 cm³/cm²—that the total article void volume increases to at least 0.620 cm³/cm² at 20% saturation (the dry void volumes related to TABLE 7 were measured according to the Percent Void Volume Increase Test Method). As can be seen in TABLE 7, each of the Example and Comparative Codes that had a dry void volume less than about 0.40 cm³/cm² achieved a first cradle intake time of less than 20 s where the total article void volume at 20% saturation reached at least 0.624 cm³/cm². In further embodiments, it may be desirable that the total article dry void volume at 20% saturation reaches at least 0.715 cm³/cm² to ensure a fast first cradle intake time. Of course, where the initial dry void volume of an absorbent article, such as articles 10, 210 of the present disclosure, is low, a main driver of reaching the threshold of 0.624 cm³/cm² or 0.715 cm³/cm² will be the rate at which the absorbent layer gains void volume during saturation—with key percentages identified previously.

It may as well be beneficial for the superabsorbent material utilized to provide an increase in void volume of the absorbent layer or layers of greater than 359% at 40% saturation, according to the Percent Void Volume Increase Test Method. This increase in absorbent layer(s) void volume may be particularly desirable where the dry void volumes of the absorbent articles, such as articles 10, 210, are low—for example less than or equal to 0.423 cm³/cm²—and where the vortex time of the superabsorbent material is less than or equal to 41 s. In the Example Codes indicated in TABLE 7, this percentage increase in void volume of the absorbent layer (these Example Codes all had a single absorbent layer) was able to produce a fast first cradle intake time of less than 20 s. Although Example Code 19 was able to produce a first cradle intake time of less than 20 s where the increase in void volume of the absorbent layer was less than 359%, it does not appear that consistent performance of a first cradle intake time of less than 20 s may be achieved with an increase in void volume of the absorbent layer or layers of greater than only 314% at 40% saturation. Again, this increase in void volume of the absorbent layer or layers may be particularly preferred where the initial dry void volume of the article is low (less than or equal to 0.423 cm³/cm²) and where the vortex time of the superabsorbent material is less than or equal to 41 s.

In more preferred embodiments, the superabsorbent material utilized should provide an increase in void volume of the absorbent layer or layers of greater than or equal to 385% at 40% saturation, according to the Percent Void Volume Increase Test Method. Comparative Example 3 was able to achieve a first cradle intake time of less than 20 s while the percentage increase in the void volume of the absorbent layer was less than 359% at 40% saturation with an initial void volume of 0.313 cm³/cm², indicating that some other factor may be influencing the first cradle intake time for superabsorbent materials with vortex times greater than 41 s. In still other embodiments, for example where a first cradle intake performance of up to 25 s is acceptable, the superabsorbent material utilized may provide an increase in void volume of the absorbent layer or layers of greater than or equal to 314% at 40% saturation, according to the Percent Void Volume Increase Test Method. Such an increase in void volume of the absorbent layer or layers may be particularly preferred where the initial dry void volume of the article is low (less than or equal to 0.423 cm³/cm²) and where the vortex time of the superabsorbent material is less than or equal to 41 s.

Looking further to TABLE 7, it may be desirable, where the dry void volume of the absorbent articles 10, 210 of the present disclosure are low—such as less than about 0.423 cm³/cm²—and where the superabsorbent material utilized has a vortex time of less than or equal to 41 s that the total article void volume increases to at least 0.841 cm³/cm² at 40% saturation. As can be seen in TABLE 7, each of the Example Codes that had a dry void volume less than about 0.40 cm³/cm² and a vortex time of less than or equal to 41 s achieved a first cradle intake time of less than 20 s where the total article void volume at 40% saturation reached at least 0.841 cm³/cm². In further embodiments, it may be desirable that the total article void volume at 40% saturation reaches at least 0.849 cm³/cm² or 0.907 cm³/cm² to ensure a fast first cradle intake time. Of course, where the initial void volume of an absorbent article, such as articles 10, 210 of the present disclosure, is low, a main driver of reaching the threshold of 0.841 cm³/cm² (or 0.849 cm³/cm² or 0.907 cm³/cm²) will be the rate at which the absorbent layer gains void volume during saturation—with key percentages identified previously. Although Comparative Example code 3 was able to achieve a first cradle intake time of less than 20 s with a dry void volume less than 0.40 cm³/cm² where the dry void volume did not reach least 0.841 cm³/cm² at 40% saturation, possibly indicating another driver of first cradle intake times where the vortex times of superabsorbent materials is greater than 41 s.

In still further embodiments, it may be preferable for the absorbent articles 10, 210 of the present disclosure to meet certain performance metrics at both 20% and 40% saturation levels. For example, it may be preferable for the absorbent articles 10, 210 having a low dry void volume, such as less than or equal to 0.423 cm³/cm², to provide an increase in void volume of the absorbent layer or layers of at least 180% at 20% saturation, according to the Percent Void Volume Increase Test Method, and an increase of at least 359% at 40% saturation, according to the Percent Void Volume Increase Test Method. It may be particularly preferred for the absorbent articles 10, 210 to meet these limitations where the dry void volume of the articles 10, 210 is low—less than 0.402 cm³/cm²—and optionally where the vortex time of the superabsorbent material in the articles 10, 210 is less than or equal to 41 s.

In further embodiments, it may be preferred that the absorbent articles 10, 210 of the present disclosure achieve particular total void volume thresholds at 20% saturation and at 40% saturation, where the articles 10, 210 begin with a low dry total void volume—for example of less than or equal to 0.423 cm³/cm²—and where the superabsorbent material utilized has a vortex time of less than or equal to 41 s. For example, in such articles 10, 210, it may be preferable for the article to have a total void volume of at least 0.624 cm³/cm² at 20% saturation and at least 0.841 cm³/cm² at 40% saturation. In further embodiments, it may be preferred for such articles to have a total void volume of at least 0.715 cm³/cm² at 20% saturation and at least 0.907 cm³/cm² at 40% saturation.

TABLE 7

| | | | | Article Void | Absorbent | Article Void | Absorbent |
|---|---|---|---|---|---|---|---|
| | First | Article | Absorbent | Volume at | Layer % Void | Volume at | Layer % Void |
| | Cradle | Dry Void | Layer Dry | 20% | Volume In- | 40% | Volume In- |
| | Intake | Volume | Void Volume | Saturation | crease at 20% | Saturation | crease at 40% |
| Code | Rate (s) | (cm³/cm²) | (cm³/cm²) | (cm³/cm²) | Saturation | (cm³/cm²) | Saturation |
| Example 3 | 22.6 | 0.331 | 0.111 | 0.620 | 160% | 0.841 | 359% |
| Example 4 | 15.1 | 0.369 | 0.111 | 0.828 | 314% | 0.907 | 385% |
| Example 6 | 12.6 | 0.423 | 0.111 | 0.795 | 235% | 0.993 | 414% |
| Example 7 | 11.2 | 0.490 | 0.111 | 0.777 | 159% | 0.997 | 357% |
| Example 12 | 12.1 | 0.402 | 0.111 | 0.845 | 299% | 1.052 | 486% |
| Example 19 | 17.4 | 0.390 | 0.111 | 0.719 | 196% | 0.849 | 314% |
| Example 20 | 13.3 | 0.460 | 0.111 | 0.750 | 161% | 0.895 | 292% |
| Example 22 | 13.8 | 0.537 | 0.111 | 0.809 | 145% | 0.946 | 268% |
| Comparative | 35.9 | 0.273 | 0.111 | 0.559 | 158% | 0.675 | 262% |
| Example 2 | | | | | | | |
| Comparative | 19.7 | 0.313 | 0.111 | 0.624 | 180% | 0.744 | 288% |
| Example 3 | | | | | | | |
| Comparative | 29.6 | 0.370 | 0.111 | 0.673 | 173% | 0.812 | 298% |
| Example 4 | | | | | | | |
| Comparative | 24.6 | 0.332 | 0.111 | 0.562 | 107% | 0.684 | 217% |
| Example 5 | | | | | | | |
| Comparative | 18.9 | 0.446 | 0.111 | 0.715 | 60% | 0.809 | 227% |
| Example 6 | | | | | | | |

The above description of low void volume absorbent articles detail particular parameters or combinations of parameters that are desirable for achieving surprising first cradle intake times at low void volumes. As described, the particular Example Codes utilized included absorbent structures formed according to aspects of the present disclosure—for example formed according to process 400. Accordingly, the absorbent articles 10, 210 of the present disclosure may not only achieve fast first cradle intake times at low void volumes as described above, the absorbent structures 101 of the articles 10, 210 may achieve the above-described performance properties related to SAM capture, wet pad integrity, pad uniformity, and z-direction adhesive distribution.

Further, it is theorized that the specific absorbent structures formed from the processes 300 or 400 may help as well to provide the surprising first cradle intake times. As described previously, the absorbent mixture 320 formed by the process 400 may include a three-dimensional mesh network having network adhesive filaments extending substantially throughout a three-dimensional space formed by the superabsorbent particles 317 with the adhesive filaments 316 oriented in a substantially random manner. The adhesive filaments may extend substantially in the lateral and longitudinal directions of the absorbent mixture 320 as well as vertically throughout a z-direction thickness of the mixture 320 where at least some of these vertically extending filaments 316 extend all the way from a first surface to a second surface of the mixture 320. Such random orientations and z-direction extension of the adhesive filaments 316 may help to prevent formation of an adhesive ‘barrier’ disposed proximate a body facing surface of the mixture 320, which could act to slow fluid intake into the structure 101.

Further, the high SAM capture and high pad uniformity features of the absorbent structures 101 of the present disclosure may help to ensure that the superabsorbent material 317 (for example, superabsorbent particles 317) within the structures 101 are disposed in a relatively even distribution and that the superabsorbent material 317 stays in a location where it was initially disposed. These features may help to ensure that the superabsorbent material 317 of the structures 101 does not clump together, providing opportunities for gel blocking and thereby slowing fluid intake speeds. Additionally, the relatively low amount of adhesive able to be utilized to achieve the SAM capture and pad uniformity features of the structures 101 as well allows for fast fluid intake and dispersion within the structure 101—as lower adhesive amounts represent lower fluid blocking by the hydrophobic adhesive. It is thought as well that the z-direction adhesive distribution formed by the processes 300, 400 may help achieve fast first cradle intake times. For example, the amount of adhesive penetration in the z-direction within the structures 101, and more specifically in the z-direction within the absorbent mixture 320 of the structures 101, means that relatively less of the applied adhesive is disposed proximate the body-facing surface of the structures 101 where it could block and prevent fluid flow to the interior of the structures 101, thereby reducing a speed of a first cradle intake rate.

Accordingly, at least some articles 10, 210 of the present disclosure may have low initial, dry void volumes (according to the Percent Void Volume Increase Test Method)—for example of less than or equal to 0.423 cm³/cm²—and have superabsorbent material 317 with vortex times of less than or equal to 41 s where the absorbent structures 101 of the articles 10, 210 have basis weights of superabsorbent material 317 of between 400 gsm and 600 gsm, and where the adhesive was applied at an add-on rate of between 4% and 5% (e.g. % Adh values of between 4% and 5%), resulting in a beneficially high SAM capture value. Alternatively, the absorbent structures 101 of such articles 10, 210 may have basis weights of superabsorbent material 317 of between 500 gsm and 600 gsm with % Adh values of between 4% and 5%, or basis weights of superabsorbent material 317 of between 500 gsm and 600 gsm and % Adh values of between 3% and 4%.

In further embodiments of the present disclosure, the articles 10, 210 of the present disclosure may have low initial, dry void volumes (according to the Percent Void Volume Increase Test Method)—for example of less than or equal to 0.423 cm³/cm²—and have superabsorbent material 317 with vortex times of less than or equal to 41 s where the absorbent structures 101 of the articles 10, 210 have superabsorbent material 317 disposed in an amount between 400 gsm and 600 gsm and one or more adhesives disposed in a combined basis weight less than 7%, by weight of the basis weight of superabsorbent material 317 to produce beneficial pad uniformity results. Alternatively, the structures 101 of these articles 10, 210 may have one or more adhesives disposed in a combined basis weight of between 4% and 7%, or between 4% and 6%, by weight of the basis weight of superabsorbent material 317. In still further embodiments of any of these such articles 10, 210, the superabsorbent material 317 may be disposed at a basis weight of between 400 gsm and 500 gsm.

Additional embodiments of articles 10, 210 of the present disclosure may include those that have low initial, dry void volumes (according to the Percent Void Volume Increase Test Method)—for example of less than or equal to 0.423 cm³/cm²—and have superabsorbent material 317 with vortex times of less than or equal to 41 s where the absorbent structures 101 of the articles 10, 210 have greater than 28% of the total amount of adhesive within the mixture 320 within the structures 101 to be located within the interior region 398 of the mixture 320. Alternatively, the structures 101 may have greater than 30.5% of the total amount of adhesive within the mixture 320 to be located within the interior region 398 of the mixture 320 or greater than 33%, or greater than 35% of the total amount of adhesive within a mixture 320 being located within the interior region 398 of the mixture 320 in further embodiments.

Of course, any of the above-described embodiments of the articles 10, 210 could further include where the superabsorbent material 317 is configured to provide an increase in void volume of the absorbent mixture or mixtures 320 of at least 180% at 20% saturation, according to the Percent Void Volume Increase Test Method. This additional feature may help provide the articles 10, 210 with a first cradle intake time of less than 20 s. For at least some of these articles 10, 210 the initial, dry void volumes may be less than or equal to 0.402 cm³/cm² (according to the Percent Void Volume Increase Test Method) and the increase in void volume of the absorbent mixture or mixtures 320 is at least 196% at 20% saturation. Alternatively, it may be that such articles 10, 210 are configured to increase in void volume upon partial saturation and achieve a void volume of at least 0.620 cm³/cm² at 20% saturation. Further articles 10, 210 may be configured to increase in void volume upon partial saturation and achieve a void volume of at least 0.715 cm³/cm² at 20% saturation.

Alternatively, any of the above-described embodiments of the articles 10, 210 could further include where the superabsorbent material 317 is configured to provide an increase in void volume of the absorbent mixture or mixtures 320 of at least 359% at 40% saturation, according to the Percent Void Volume Increase Test Method. This feature as well may help provide the articles 10, 210 with a first cradle intake time of less than 20 s. Alternatively, it may be that such articles 10, 210 are configured to increase in void volume upon partial saturation and achieve a void volume of at least 0.841 cm³/cm² at 40% saturation. Further articles 10, 210 may be configured to increase in void volume upon partial saturation and achieve a void volume of at least 0.849 cm³/cm² or at least 0.907 cm³/cm² at 40% saturation.

Of course, the above-described embodiments of the articles 10, 210 could further include where the superabsorbent material 317 is configured to provide an increase in void volume of the absorbent mixture or mixtures 320 of at least 196% at 20% saturation, according to the Percent Void Volume Increase Test Method, and an increase of at least 359% at 40% saturation, according to the Percent Void Volume Increase Test Method. Alternatively, it may be that such articles 10, 210 are configured to increase in void volume upon partial saturation and achieve a void volume of at least 0.624 cm³/cm² at 20% saturation and at least 0.841 cm³/cm² at 40% saturation. In further embodiments, it such articles may be configured to achieve a void volume of at least 0.715 cm³/cm² at 20% saturation and at least 0.907 cm³/cm² at 40% saturation.

It should be understood that while the above-described embodiments indicated that the absorbent articles 10, 210 may have a dry void volume of than or equal to 0.423 cm³/cm² (according to the Percent Void Volume Increase Test Method) and superabsorbent material 317 with vortex times of less than or equal to 41 s, further embodiments may have even lower ranges of dry void volumes and/or superabsorbent material 317 with faster vortex times. For example, it may be more preferable for the above-described articles 10, 210 to have a dry void volume of less than or equal to 0.40 cm³/cm² (according to the Percent Void Volume Increase Test Method), or less than or equal to 0.35 cm³/cm² (according to the Percent Void Volume Increase Test Method), or less than or equal to 0.30 cm³/cm² (according to the Percent Void Volume Increase Test Method), or less than or equal to 0.25 cm³/cm² (according to the Percent Void Volume Increase Test Method), or less than or equal to 0.20 cm³/cm² (according to the Percent Void Volume Increase Test Method). Alternatively, or additionally, it may be preferable for the above-described articles 10, 210 to have superabsorbent material 317 which have vortex times of less than or equal to 29 s, or less than or equal to 18 s, or less than or equal to 14 s.

### SAM Capture Test Method

Individual sample absorbent bodies are first obtained, whether by deconstruction of a commercially available product or by obtaining individual structures directly from a manufacturing line prior to incorporation into a product. If obtained from a commercially available product, typical product deconstruction methods should be used to obtain only the absorbent body, such as the use of a freeze spray or other equivalent products which helps to deactivate any adhesive laminating the various layers of the product together, allowing for easier separation of the layers, and/or scissors to cut open one or more portions of the product. If obtained directly from a manufacturing line, the sample absorbent bodies should be left to cure for a minimum of 24 hours.

Once the sample absorbent bodies are ready, each individual sample should be weighed, and the weights should be recorded. Next, each sample structure is peeled apart, preferably over a garbage can or the like to capture any material fall out. The samples may be peeled apart by grasping one of the outside web materials in each hand, at one end of the structure, and pulling apart in a peeling motion. Once pulled apart, the separated webs are given a slight shake over the garbage can and then placed back on the scale for a second weighing, which is recorded.

A difference in the first recorded weight of a sample and a second recorded weight of the sample represents the amount of superabsorbent material lost. This difference value may then be used to determine a percentage of the total amount of superabsorbent material retained. In the present disclosure, since the web materials, the basis weights of the deposited superabsorbent material, and the adhesive add-on amounts were the same for the compared sample structures, this difference value was simply divided by the first record weight of a sample to arrive at the reported percent of superabsorbent material retained value. However, where comparing un-like samples, the basis weights and sizes of the web materials may be taken into account—for example by subtracting the total weight of the web materials of a sample from the first and second recorded weights. The total weight of the adhesives may be considered as generally negligible to the percentage retained value determination and therefore not accounted for separately.

### Wet Pad Integrity Test Method

Individual sample absorbent bodies are first obtained, whether by deconstruction of a commercially available product or by obtaining individual structures directly from a manufacturing line prior to incorporation into a product. If obtained from a commercially available product, typical product deconstruction methods should be used to obtain only the absorbent body, such as the use of a freeze spray or other equivalent products which helps to deactivate any adhesive laminating the various layers of the product together, allowing for easier separation of the layers, and/or scissors to cut open one or more portions of the product. If obtained directly from a manufacturing line, the sample absorbent bodies should be left to cure for a minimum of 24 hours.

Once obtained, target locations are marked for each sample. The target locations are marked 8.5 cm toward the front edge of the sample. The front edge of the sample is the edge located closest to the front of a product, if removed from a product, or toward the edge that would be placed closest to the front of a product if the sample was obtained directly from the manufacturing line. The product should then be adhered to a lightbox or other suitable work surface. The sample may be adhered with double side tape or the like positioned at front and/or rear edges of the sample.

Next, a plastic tube, having a length of 152 mm and a diameter of 51 mm (with a wall thickness of 3.5 mm and an internal diameter of 44 mm), is centered at the target location. A plastic funnel is placed at the top of the plastic tube and 100 ml of 0.9% blue colored saline was poured into the funnel. Care should be taken so as to not apply any pressure to the surface of the sample while holding the tube in place. Additionally, the funnel spout should be angled toward a wall of the tube so that the saline flows down the side of the tube before contacting the surface of the sample. Once the fluid has been poured into the funnel, a 5-minute timer is set.

After the 5 minutes, the sample is then hung from a product shaker machine. The product shaker machine consists of a simple frame with linear actuator attached to the top of the frame and oriented in the vertical direction. A 12-inch (305 mm) long horizontal bar is connected directly to the actuator and two product clips are attached to the horizontal bar. The sample absorbent body front edge is connected to the product shaker machine through the clips. The product shaker is then switched on and the number of shakes counted. The linear actuator is configured to move the 12-inch bar up and down a total linear distance of 1-inch (25.4 mm) per half-stroke (one movement down or one movement up). A full-stroke movement is counted as one shake. Many commercially available linear actuators may be used as part of such a product shaker machine. For example, commercially available 12V or 24V actuators having a 25 mm stroke and around a fifty-pound rating along with actuating on the order of 30 mm per second may be particularly suitable actuators. Any suitable simple drive circuitry can be utilized to operate the linear actuator through extend and retract cycles. While the product shaker is on, the sample is observed for any partial breaks, which constitute any crack or gap appearing in the sample. Once the first partial break is observed, the number of shakes is noted and the product shaker machine turned off. If no partial cracks were observed by fifty shakes, the test is stopped and a value of 50 shakes was recorded for the sample.

### Pad Uniformity Test Method

The cross-machine direction (CD) gray-level variation properties of thin, fluffless absorbent fibrous webs, including structures 101 formed according to the methods 300 and 400 according to the present disclosure and including structures formed according to the Nordson process, can be determined using an image analysis method described herein. In this context, CD gray-level variations for thin fluffless absorbent fibrous webs provide an indication of a uniformity of distribution of the adhesives and superabsorbent particles throughout the webs. For instance, webs having a lower CD gray level variation may be considered to have adhesives and superabsorbent particles disposed relatively more uniformity through the webs, because the amount light passing through the webs is relatively more uniform throughout the webs as compared to webs having a relatively higher CD gray level variation, as will be explained in more detail below.

The method for determining the CD gray level variation includes using diffuse, transmitted light which passes through the web and is detected by a camera. The camera may specifically be a CCD camera such as a Leica Microsystems DFC 310 camera available from Leica Microsystems of Heerbrugg, Switzerland. The camera may be mounted to a macro-viewer camera stand, such as a Polaroid MP4 macro-viewer camera stand, or equivalent. An adjustable lens assembly, such as a Nikon 35-mm lens with an f-stop setting of 4, is attached to the camera via a c-mount connection. The camera is set in monochrome mode and a flat field correction is performed on a white background prior to analysis.

An auto stage including a transparent support is placed on the upper surface of the macro-viewer between the video camera and a diffuse light source of the macro-viewer. The auto stage may be a Design Components Incorporated Model HM-1212 or equivalent. The diffuse, transmitted lighting may be provided by four LED tube lights (EMC-9 watt, dimmable) that are disposed beneath the auto stage, and the macro-viewer includes a diffusing plate located between the LED lights auto stage. The LED light's illumination level can be controlled via a common voltage controller equipped with a knob or slider for adjustments.

Two black masks are placed on the transparent support of the auto stage and spaced three inches apart and having a long dimension running to the front and rear of the auto stage (e.g. toward and away from the camera stand of the macro-viewer). A web sample is placed flat onto the transparent support and centered between the black masks so that only the central region of the sample is illuminated. The web sample is oriented similarly to the black masks with the longitudinally extending side edges (e.g. the long dimension side edges) of the web sample running toward and away from the camera stand. The camera and lens assembly are mounted onto the macro-viewer camera stand at such a distance above the sample that provides an image field-of-view size of approximately 4 and a half inches across a width of the auto stage (e.g. perpendicular to the longitudinally extending side edges of the sample).

Analysis is performed by placing a fibrous web sample onto the auto-stage as described above under the optical axis of the camera and lens assembly. The specimen must lay flat and care is taken to ensure that wrinkles or similar deformities are removed or avoided. An image analysis software package is used to monitor and adjust the illumination level, acquire an image and then perform the measurements for determining gray-level variation. For the analysis described, a Leica Microsystems LAS software platform is used along with the custom-written algorithm Gray Level of CD Variation (Activ Tech)−1 to monitor and adjust the light level of illumination for each sample and perform gray-level variation measurements. The algorithm, which is run using the LAS Macro Editor platform, is shown below.

```
NAME = Gray Level of CD Variation (Activ Tech) − 1
PURPOSE = Measures gray-level values of grid elements across CD
CONDITIONS = DFC 310 camera; 35 mm adj lens (f/4); diffuse transmitted light; pole = 76 cm
AUTHOR = D. G. Biggs
DATE = February 21, 2020
OPEN DATA FILES & SET VARIABLES
Pause Text ( “Enter EXCEL data file and image file prefix names now.” ) Input ( TITLE$ )
OPENFILE$ = “C:\Data\102888 − Graverson\”+TITLE$+“.xls”
Open File ( OPENFILE$, channel #CHAN )
SET Graphics VARIABLES
GRAPHNX = 6
GRAPHNY = 2
GRAPHWID = 790
GRAPHHGHT = 118
GRAPHORGX = 270
GRAPHORGY = 100
GRAPHTHIK = 2
GRAPHORNT = 0
GRAPHOUT = 0
COUNT = 0
SET-UP AND CALIBRATION
Calvalue = 0.0833 mm/px
CALVALUE = 0.0833
Calibration ( Local )
Enter Results Header
File Results Header ( channel #1 )
File Line ( channel #1 )
File Line ( channel #1 )
Image frame ( x 0, y 0, Width 1392, Height 1040 )
Measure frame ( x 260, y 72, Width 806, Height 962 )
SAMPLE LOOP
For (SAMPLE = 1 to 3, step 1)
  PauseText (“Place sample onto stage.”)
Image Setup DC Twain [PAUSE] (Camera 1, AutoExposure Off, Gain 0.00, ExposureTime 15.69 msec,
Brightness 0, Lamp 49.99)
  Stage ( Define Origin )
  Stage ( Scan Pattern, 1 × 3 fields, size 102000.000000 × 96570.000000 )
  IMAGE LOOP
  For ( IMAGE = 1 to 3, step 1 )
  ACQUIRE IMAGE
  Image Setup DC Twain [PAUSE] ( Camera 1, AutoExposure Off, Gain 0.00,
  ExposureTime 15.69 msec, Brightness 0, Lamp 49.99 )
  Colour Transform ( Mono Mode )
  Acquire ( into Image0 )
  COUNT = COUNT+1
  -- The following line is the image storage location on the hard drive.
  ACQFILE$ = “C:\Images\102888 − Graverson\”+TITLE$+“_”+STR$(COUNT)+“.tif”
  Write image ( from ACQOUTPUT into file ACQFILE$ )
  GRAPHORGY = 100
  ANALYSIS LOOP
  For ( ANALYSIS = 1 to 4, step 1 )
  BINARY PROCESSING
  Graphics ( Inverted Grid, GRAPHNX × GRAPHNY Lines, Grid Size GRAPHWID ×
  GRAPHHGHT, Origin GRAPHORGX × GRAPHORGY,
  Thickness GRAPHTHIK, Orientation GRAPHORNT, to GRAPHOUT Cleared )
  Display ( Image0 (on), frames (on,on), planes (0,off,off,off,off,off), lut 0, x 0, y 0, z 0,
  Reduction off )
  MEASURE FEATURE GRAY LEVEL
  Measure feature ( plane Binary0, 32 ferets, minimum area: 4, grey image: Image0 )
  Selected parameters: X FCP, Y FCP, MeanGrey, GreyVarianc
  File Feature Results ( channel #1 )
  File Line ( channel #1 )
  File Line ( channel #1 )
  File Line ( channel #1 )
  MEASURE GL %COV
  MGREYIMAGE = 0
  MGREYMASK = 0
  Measure Grey ( plane MGREYIMAGE, mask MGREYMASK,
  histogram into GREYHIST(256), stats into GREYSTATS(2) )
  Selected parameters: MeanGrey, Std Dev
  MEANGREY = GREYSTATS(1)
  GREYSDEV = GREYSTATS(2)
  GLPERCCOV = GREYSDEV/MEANGREY*100
  File ( “GL %COV = ”, channel #1 )
  File ( GLPERCCOV, channel #1, 2 digits after ‘.’ )
  File Line ( channel #1 )
  File Line ( channel #1 )
  GRAPHORGY = GRAPHORGY+250
  Next (ANALYSIS)
  Stage (Step, Wait until stopped + 550 msecs)
  Next (IMAGE)
Next (SAMPLE)
  Close File (channel #1)
END
```

Once the algorithm is executed using the Leica software, the analyst will be prompted to enter an EXCEL data file sample and image file prefix name which will be used to store measurement data as well as the image files acquired. Both will be saved onto the computer hard drive. Next, the analyst will be prompted to properly place the sample on the sample stand so that the region to be measured is located between the two black masks. The top edge of the sample should also be located at least an inch or more above the top edge of the field-of-view image. After the sample is properly placed, and the analyst continues the algorithm, the analyst will then be prompted to adjust the illumination level so that the displaying white level is set to approximately 0.95. Once set, the software algorithm then proceeds automatically to acquire and save the image and then perform image processing and analysis steps by placing a five-box grid on the image spanning the width of the sample (e.g. in the CD) and making mean gray and gray variation measurements within each individual box. This data is then exported to the previously named EXCEL spreadsheet and the same grid is again used to measure mean and standard deviation of gray level under the entire grid at one time. From this data, the algorithm then calculates the corresponding gray-level percent coefficient-of-variation (GL % COV) and exports this data to the EXCEL spreadsheet. The GL % COV is calculated as follows:

$$GL \% COV = gray - level standard deviation / mean gray - level \times 100 \% \qquad ( 1 )$$

The CD-spanning measurement grid is nearly 66 mm across and is subdivided into five equally sized boxes. The mean gray and gray variation measurements are made for each box, while the GL % COV measurement is made for all boxes combined. Once the first measurements are made near the top of an image, the algorithm then moves the grid down 2.1 cm and a second set of measurements are made on the same image and exported to the EXCEL spreadsheet. This is repeated two more times, so that a total of four, CD-running regions are measured for each image. The algorithm then instructs the auto-stage to move the sample longitudinally by 8.2 cm, and the process of setting the white level for the next image begins again. For each sample replicate, three separate images will be acquired and analyzed. A total of three sample replicate pieces are then analyzed per sample.

For gray-level variation measurements, the five measurements made for each grid location are subsequently averaged in the EXCEL spreadsheet. These averages are then accumulated over 36 different grid positions (i.e. 3 replicates×3 images×4 CD locations=36 CD locations) for comparing different samples. After results are acquired from different samples, they can be compared to one another by performing a basic statistical analysis, such as a Student's T analysis at the 90% confidence level.

### Adhesive Distribution Test Method

Samples to be imaged are first stained with osmium tetroxide fume so that the adhesive selectively absorbs the osmium in sufficient quantities to allow it to be more easily contrasted from the super absorbent and polymeric fibers during Micro-CT imaging. A sample is stained by placing it in a closable, air-tight chamber to which a small vial of osmium tetroxide is added. The chamber is then immediately sealed and the osmium tetroxide is allowed to interact with the sample for at least 24 hours. Since osmium tetroxide is highly toxic, the staining procedure is carried out in a fume hood. After the 24 hour period, the adhesive should become blackened in appearance. After re-opening the chamber, it is allowed to air out in the hood for another 24 hours to ensure any unreacted osmium tetroxide is allowed to harmlessly escape. After the second 24 hour period, the sample is now ready to be imaged in the Micro-CT,

A Bruker SkyScan Model 1272 Micro-CT, or equivalent, is used to image a portion of the stained sample. Example X-ray scanning conditions include the following:
Voltage (kV)=35
Current (uA)=231
Image Pixel Size (um)=8.0
Rotation Step (deg)=0.20
Frame Averaging=5
   The sample piece must be oriented so that machine-direction length is held in the vertical position during the scanning process. After initial x-ray scanning, the rotational x-ray images are then reconstructed using Bruker's NRecon software, or equivalent on a different vendor's system. The gray-scale reconstructed image slices are used for the adhesive distribution analysis.

The image analysis software platform used to perform the adhesive distribution measurements may be a QWIN Pro (Version 3.2.1) available from Leica Microsystems, having an office in Heerbrugg, Switzerland. The custom-written image analysis algorithm ‘Z-Adhesive Distribution’ was used to process and perform measurements of grayscale Micro-CT images using Quantimet User Interactive Programming System (QUIPS) language. The custom image analysis algorithm shown below was performed directly on the gray-scale reconstructed image slices that were stored on a storage device. The custom image analysis algorithm is shown below.

```
NAME: Z-Adhesive Distribution
  PURPOSE: Measures z-distribution of osmium stained adhesive on ActivTech/Blizzard Substrates
  CONDITIONS: Images acquired on the Bruker SkyScan 1272 Micro-CT
  DATE: August 12, 2020
  AUTHOR: D. G. Biggs
  SET-UP
  Clear Accepts
DATA FILES OPENED
Open File ( C:\Data\102888 − Graverson\totdistribution.xls, channel #2 )
  Open File ( C:\Data\102888 − Graverson\adhesivedistribution.xls, channel #1 )
  Configure ( Image Store 1968 × 504, Grey Images 201, Binaries 32 )
  -- Calvalue = 8.00 um/px
  CALVALUE = 8.00
  Calibrate ( CALVALUE CALUNITS$ per pixel )
  Measure frame ( x 160, y 2, Width 1600, Height 502 )
  Image frame ( x 0, y 0, Width 1968, Height 504 )
  Enter Results Header
  File Results Header ( channel #1 )
  File Line ( channel #1 )
  File Results Header ( channel #2 )
  File Line ( channel #2 )
  PauseText ( “Enter sample image file prefix name.” )
  Input ( TITLE$ )
  File ( TITLE$, channel #1 )
  File Line ( channel #1 )
  For ( IMAGE = 100 to 900, step 100 )
  Clear Feature Histogram #1
  Clear Feature Histogram #3
  DEFINE BINARY GRAPHICS VARIABLES
  GRAPHORGX = 250
  IMAGE ACQUISITION AND DETECTION
  ACQOUTPUT = 0
-- Location of Micro-CT images to be analyzed
  ACQFILE$ = “C:\Images\102888 − Graverson\Code 2 − Blizzard Tech
Osmium\”+TITLE$+“”+STR$(IMAGE)+“.JPG”
  Read image ( from file ACQFILE$ into ACQOUTPUT )
  Colour Transform ( Mono Mode )
  -- Detect all material
  Detect ( whiter than 33, from Image0 into Binary0 )
  IMAGE PROCESSING
  PauseText ( “Accept the primary structure and exclude any outlying debris.” )
  Binary Edit [PAUSE] ( Accept from Binary0 to Binary1, nib Fill, width 2 )
  Binary Amend ( Open from Binary1 to Binary1, cycles 1, operator Disc, edge erode on )
  Binary Amend ( Close from Binary1 to Binary2, cycles 120, operator Disc, edge erode on )
  Binary Identify ( FillHoles from Binary2 to Binary3 )
  Binary Amend ( Open from Binary3 to Binary4, cycles 5, operator Disc, edge erode on )
  BOLEAN AND MEASUREMENT
  For ( BINGRAPH = 1 to 26, step 1 )
    GRAPHORGY = 2
    GRAPHNX = 1
    GRAPHNY = 1
    GRAPHWID = 50
    GRAPHHGHT = 502
    GRAPHTHIK = 1
    GRAPHORNT = 0
    GRAPHOUT = 13
    Graphics ( Inverted Grid, GRAPHNX × GRAPHNY Lines, Grid Size GRAPHWID × GRAPHHGHT, Origin
GRAPHORGX × GRAPHORGY,
      Thickness GRAPHTHIK, Orientation GRAPHORNT, to GRAPHOUT Cleared )
    Binary Logical ( C = A AND B : C Binary5, A Binary4, B Binary13 )
    CENTER YPOS
    Measure feature ( plane Binary5, 32 ferets, minimum area: 10, grey image: Colour0 )
      Selected parameters: UserDef1, YCentroid
    Feature Expression ( UserDef1 ( all features ), title CalcA = (PYCENTROID(FTR)-252) )
    GREYUTILIN = 0
    GREYUTILOUT = 1
    -- Shift Grey Image
    If ( PUSERDEF1(FTR)<0 )
    DISTANCE = (PUSERDEF1(FTR)**2)**0.5
    SHIFT.SIZE = DISTANCE
    SHIFT.DIRN = 270
    Grey Util ( Shift GREYUTILIN to GREYUTILOUT by SHIFT.SIZE at SHIFT.DIRN degs )
    Endif
    If ( PUSERDEF1(FTR)>0 )
    DISTANCE = PUSERDEF1(FTR)
    SHIFT.SIZE = DISTANCE
    SHIFT.DIRN = 90
    Grey Util ( Shift GREYUTILIN to GREYUTILOUT by SHIFT.SIZE at SHIFT.DIRN degs )
    Endif
    If ( PUSERDEF1(FTR)=0 )
    Grey Util ( Copy Image0 to Image1 )
    Endif
    Display ( Image0 (on), frames (on,on), planes (off,off,off,off,off,off), lut 0, x 0, y 0, z 1, Reduction off )
    DETECT AFTER CENTERING
    -- Detect adhesive
    Detect ( whiter than 84, from Image1 into Binary10 )
    Binary Amend ( Close from Binary10 to Binary10, cycles 1, operator Disc, edge erode on )
    Binary Amend ( Open from Binary10 to Binary11, cycles 1, operator Disc, edge erode on )
    -- Detect all material
    Detect ( whiter than 33, from Image1 into Binary0 )
    Binary Amend ( Close from Binary0 to Binary0, cycles 1, operator Disc, edge erode on )
    Binary Amend ( Open from Binary0 to Binary0, cycles 1, operator Disc, edge erode on )
    MEASURE ADHESIVE Z-DISTRIBUTION
    GRAPHORGY = 2
    GRAPHNX = 1
    GRAPHNY = 1
    GRAPHWID = 50
    GRAPHHGHT = 502
    GRAPHTHIK = 1
    GRAPHORNT = 0
    GRAPHOUT = 12
    Graphics ( Inverted Grid, GRAPHNX × GRAPHNY Lines, Grid Size GRAPHWID × GRAPHHGHT, Origin
GRAPHORGX × GRAPHORGY,
      Thickness GRAPHTHIK, Orientation GRAPHORNT, to GRAPHOUT Cleared )
    Binary Logical ( C = A AND B : C Binary6, A Binary12, B Binary11 )
    Measure feature ( plane Binary6, 32 ferets, minimum area: 10, grey image: Image1 )
      Selected parameters: Area, UserDef2, YCentroid
    Feature Expression ( UserDef2 ( all features ), title YFEAT = PYCENTROID(FTR)*CALVALUE )
    Feature Histogram #1 ( Y Param Area, X Param UserDef2, from 0. to 4032., linear, 40 bins )
    Feature Histogram #2 ( Y Param Area, X Param UserDef2, from 0. to 4032., linear, 40 bins )
    MEASURE TOTAL MATERIAL Z-DISTRIBUTION
    Binary Logical ( C = A AND B : C Binary7, A Binary12, B Binary0 )
    Measure feature ( plane Binary7, 32 ferets, minimum area: 10, grey image: Image1 )
      Selected parameters: Area, X FCP, Y FCP, UserDef2, YCentroid
    Feature Expression ( UserDef2 ( all features ), title YFEAT = PYCENTROID(FTR)*CALVALUE )
    Feature Histogram #3 ( Y Param Area, X Param UserDef2, from 0. to 4032., linear, 40 bins )
    Feature Histogram #4 ( Y Param Area, X Param UserDef2, from 0. to 4032., linear, 40 bins )
    GRAPHORGX = GRAPHORGX+50
  Next ( BINGRAPH )
  Display Feature Histogram Results ( #2, horizontal, differential, bins + graph (Y axis linear), statistics )
    Data Window ( 10, 871, 640, 300 )
  Display Feature Histogram Results ( #4, horizontal, differential, bins + graph (Y axis linear), statistics )
    Data Window ( 962, 880, 640, 300 )
  FILE ADHESIVE AND MATERIAL HISTOGRAMS FOR CURRENT IMAGE
  File Feature Histogram Results ( #1, differential, statistics, bin details, channel #1 )
  File Line ( channel #1 )
  File Feature Histogram Results ( #3, differential, statistics, bin details, channel #2 )
  File Line ( channel #2 )
  File Line ( channel #2 )
  MEASURE MEAN SUBSTRATE THICKNESS
  MFLDIMAGE = 4
  Measure field ( plane MFLDIMAGE, into FLDRESULTS(1), statistics into FLDSTATS(7,1) )
    Selected parameters: Area
  MEANTHICK = FLDRESULTS(1)/(CALVALUE*1330)
  File ( “Mean Substrate Thickness (um) = ”, channel #1 )
  File ( MEANTHICK, channel #1, 2 digits after ‘.’ )
  File Line ( channel #1 )
  File Line ( channel #1 )
  Next ( IMAGE )
  FILE CUMMULATIVE ADHESIVE AND MATERIAL HISTOGRAMS FOR CURRENT SLIDE
  File Feature Histogram Results ( #2, differential, statistics, bin details, channel #1 )
  File Feature Histogram Results ( #4, differential, statistics, bin details, channel #2 )
  CLOSE DATA FILES
  Close File ( channel #1 )
  Close File ( channel #2 )
END
```

The adhesive distribution in the z-direction data are exported directly to an EXCEL® spreadsheet. Individual adhesive and total material z-distribution histograms are exported for data acquired from each of the analyzed slices of the micro-CT image as well as a cumulative histogram for data from all nine slices. These latter cumulative histograms were used for calculating the percentage of adhesive in each one-third layer of the thickness of the micro-CT image for a single slice. The area units are shown in the histogram are in square microns. In order to determine the histogram location of the top and bottom surface boundaries of the material, a 95 percent of total area rule was used on the total material histogram. In other words, when approaching the top and bottom material edges of the histogram, the surface boundary was considered to be the first histogram bin when a minimum of 2.5 percent material area had been encountered. These bin boundaries were then transposed over to the adhesive only cumulative histogram to determine the percentages of adhesive area present in the top, middle and bottom one-third histogram bins, inclusive of the calculated boundary bins. In cases where the number of bins was not evenly divisible by three (e.g. 8, 10, 14, etc.), a rotation technique was used to calculate adhesive percentages in each one-third layer of the material. For example, in the first encounter of a fourteen bin thickness, the top layer was four bins, the middle five, and the bottom five. During the next encounter, the top layer was five bins, the middle four, and the bottom five. If a third encounter occurs, the bottom layer would have one less or one more bin than the top and middle. If a fourth encounter occurs, the top layer again becomes the one containing one less or one more bin than the other two layers. This rotating method continues as required by the data.

The final sample mean adhesive percentage values for each one-third layer of z-distribution depth is based on an N=7 analysis from seven, separate, subsample regions each possessing four adjacently cut cross-sections. A comparison between different samples can be performed using a Student's T analysis at the 90 percent confidence level.

### Vortex Time Test Method:

The vortex time is the amount of time in seconds required for a predetermined mass of superabsorbent particles to close a vortex created by stirring 50 milliliters of 0.9 percent by weight sodium chloride solution at 600 revolutions per minute on a magnetic stir plate. The time it takes for the vortex to close is an indication of the free swell absorbing rate of the particles. The vortex time test can be performed at a temperature is 23° C.±2° C. and relative humidity of 50%±5% according to the following procedure:
(1) Measure 50 milliliters (±0.01 milliliter) of 0.9 percent by weight sodium chloride solution into a 100-milliliter beaker.
(2) Place a 7.9 millimeters×32 millimeters TEFLON® covered magnetic stir bar without rings (such as that commercially available under the trade designation S/P® brand single pack round stirring bars with removable pivot ring) into the beaker.
(3) Program a magnetic stir plate (such as that commercially available under the trade designation DATAPLATE® Model #721) to 600 revolutions per minute.
(4) Place the beaker on the center of the magnetic stir plate such that the magnetic stir bar is activated. The bottom of the vortex should be near the top of the stir bar. The superabsorbent particles are pre-screened through a U.S. standard #30 mesh screen (0.595 millimeter openings) and retained on a U.S. standard #50 mesh screen (0.297 millimeter openings).
(5) Weigh out the required mass of the superabsorbent particles to be tested on weighing paper.
(6) While the sodium chloride solution is being stirred, quickly pour the absorbent polymer to be tested into the saline solution and start a stopwatch. The superabsorbent particles to be tested should be added to the saline solution between the center of the vortex and the side of the beaker.
(7) Stop the stopwatch when the surface of the saline solution becomes flat and record the time. The time, recorded in seconds, is reported as the vortex time.

### Free-Swell Gel Bed Permeability Test Method (GBP Test Method):

As used herein, the Free Swell Gel Bed Permeability Test Method (GBP Test Method) determines the permeability of a swollen bed of superabsorbent material under what is commonly referred to as “free swell” conditions. The term “free swell” means that the superabsorbent material is allowed to swell without a swell restraining load upon absorbing test solution as will be described. This test is described in U.S. Pat. No. 8,021,998 to Qin et al., which is incorporated herein by reference thereto. For instance, a test apparatus can be employed that contains a sample container and a piston, which can include a cylindrical LEXAN shaft having a concentric cylindrical hole bored down the longitudinal axis of the shaft. Both ends of the shaft can be machined to provide upper and lower ends. A weight can rest on one end that has a cylindrical hole bored through at least a portion of its center. A circular piston head can be positioned on the other end and provided with a concentric inner ring of seven holes, each having a diameter of about 0.95 cm, and a concentric outer ring of fourteen holes, each having a diameter of about 0.95 cm. The holes are bored from the top to the bottom of the piston head. The bottom of the piston head can also be covered with a biaxially stretched mesh stainless steel screen. The sample container can contain a cylinder and a 100-mesh stainless steel cloth screen that is biaxially stretched to tautness and attached to the lower end of the cylinder. Superabsorbent particles can be supported on the screen within the cylinder during testing.

The cylinder can be bored from a transparent LEXAN rod or equivalent material, or it can be cut from a LEXAN tubing or equivalent material, and has an inner diameter of about 6 cm (e.g., a cross-sectional area of about 28.27 cm²), a wall thickness of about 0.5 cm and a height of approximately 5 cm. Drainage holes can be formed in the sidewall of the cylinder at a height of approximately 7.95 cm above the screen to allow liquid to drain from the cylinder to thereby maintain a fluid level in the sample container at approximately 7.95 cm above the screen. The piston head can be machined from a LEXAN rod or equivalent material and has a height of approximately 16 mm and a diameter sized such that it fits within the cylinder with minimum wall clearance but still slides freely. The shaft can be machined from a LEXAN rod or equivalent material and has an outer diameter of about 2.22 cm and an inner diameter of about 0.64 cm. The shaft upper end is approximately 2.54 cm long and approximately 1.58 cm in diameter, forming an annular shoulder to support the annular weight. The annular weight, in turn, has an inner diameter of about 1.59 cm so that it slips onto the upper end of the shaft and rests on the annular shoulder formed thereon. The annular weight can be made from stainless steel or from other suitable materials resistant to corrosion in the presence of the test solution, which is 0.9 wt. % sodium chloride solution in distilled water. The combined weight of the piston and annular weight equals approximately 596 grams, which corresponds to a pressure applied to the sample of about 0.3 pounds per square inch, or about 20.7 dynes/cm², over a sample area of about 28.27 cm². When the test solution flows through the test apparatus during testing as described below, the sample container generally rests on a 16-mesh rigid stainless steel support screen. Alternatively, the sample container can rest on a support ring diametrically sized substantially the same as the cylinder so that the support ring does not restrict flow from the bottom of the container.

To conduct the GBP Test Method under “free swell” conditions, the piston, with the weight seated thereon, is placed in an empty sample container and the height from the bottom of the weight to the top of the cylinder is measured using a caliper or suitable gauge accurate to 0.01 mm. The height of each sample container can be measured empty and which piston and weight is used can be tracked when using multiple test apparatus. The same piston and weight can be used for measurement when the sample is later swollen following saturation. The sample to be tested is prepared from superabsorbent particles that are prescreened through a U.S. standard 30-mesh screen and retained on a U.S. standard 50-mesh screen. The particles can be prescreened by hand or automatically.

Approximately 0.9 grams of the sample is placed in the sample container, and the container, without the piston and weight therein, is then submerged in the test solution for a time period of about 60 minutes to saturate the sample and allow the sample to swell free of any restraining load. At the end of this period, the piston and weight assembly is placed on the saturated sample in the sample container and then the sample container, piston, weight, and sample are removed from the solution. The thickness of the saturated sample is determined by again measuring the height from the bottom of the weight to the top of the cylinder, using the same caliper or gauge used previously provided that the zero point is unchanged from the initial height measurement. The height measurement obtained from measuring the empty sample container, piston, and weight is subtracted from the height measurement obtained after saturating the sample. The resulting value is the thickness, or height “H” of the swollen sample.

The permeability measurement is initiated by delivering a flow of the test solution into the sample container with the saturated sample, piston, and weight inside. The flow rate of test solution into the container is adjusted to maintain a fluid height of about 7.95 cm above the bottom of the sample container. The quantity of solution passing through the sample versus time is measured gravimetrically. Data points are collected every second for at least twenty seconds once the fluid level has been stabilized to and maintained at about 7.95 cm in height. The flow rate Q through the swollen sample is determined in units of grams/second (g/s) by a linear least-square fit of fluid passing through the sample (in grams) versus time (in seconds). The permeability is obtained by the following equation:

$$K = ( 1 \div 0.98692 \times {10}^{- 8} ) * [ [ Q * H * Mu ] / [ A * Rho * P ] ]$$

where:
   K=Permeability (darcys),
   Q=flow rate (g/sec),
   H=height of sample (cm),
   Mu=liquid viscosity (poise) (approximately 1 centipoise for the test solution used with this test),
   A=cross-sectional area for liquid flow (cm²),
   Rho=liquid density (g/cm³) (approximately 1 g/cm³ for the test solution used with this Test), and
   P=hydrostatic pressure (dynes/cm²) (normally approximately 3,923 dynes/cm²), which can be calculated from Rho*g*h, where Rho=liquid density (g/cm³), g=gravitational acceleration, nominally 981 cm/sec², and h=fluid height, e.g., 7.95 cm.

A minimum of three samples should be tested and the results averaged to determine the free swell gel bed permeability of the sample. The samples should be tested at 23° C. and 50% relative humidity.

### Absorbency Under Load (AUL) Test Method:

The absorbent capacity of superabsorbent particles can be measured using an Absorbency Under Load (“AUL”) Test Method, which is a well-known test for measuring the ability of superabsorbent particles to absorb a 0.9 wt. % solution of sodium chloride in distilled water at room temperature (test solution) while the material is under a load. For example, 0.16 grams of superabsorbent particles can be confined within a 5.07 cm2 area of an Absorbency Under Load (“AUL”) cylinder under a nominal pressure of 0.01 psi, 0.3 psi, or 0.9 psi. The sample is allowed to absorb the test solution from a dish containing excess fluid. At predetermined time intervals, a sample is weighed after a vacuum apparatus has removed any excess interstitial fluid within the cylinder. This weight versus time data is then used to determine the Absorption Rates at various time intervals.

The AUL test apparatus is measured according to EDANA recommended test method WSP 242.3 which is similar to a GATS (gravimetric absorbency test system), available from M/K Systems, as well as the system described by Lichstein at pages 129-142 of the INDA Technological Symposium Proceedings, March 1974. A ported disk is also utilized having ports confined within a 2.5-centimeter diameter area. The resultant AUL is stated as grams of liquid retained per gram weight of the sample (g/g).

To carry out the test, the following steps may be performed:
(1) Wipe the inside of the AUL cylinder with an anti-static cloth, and weigh the cylinder, weight and piston;
(2) Record the weight as CONTAINER WEIGHT in grams to the nearest milligram;
(3) Slowly pour the 0.16±0.005 gram sample of the superabsorbent particles into the cylinder so that the particles do not make contact with the sides of the cylinder or it can adhere to the walls of the AUL cylinder;
(4) Weigh the cylinder, weight, piston, and superabsorbent particles and record the value on the balance, as DRY WEIGHT in grams to the nearest milligram;
(5) Gently tap the AUL cylinder until the superabsorbent particles are evenly distributed on the bottom of the cylinder;
(6) Gently place the piston and weight into the cylinder;
(7) Place the test fluid (0.9 wt. % aqueous sodium chloride solution) in a fluid bath with a large mesh screen on the bottom;
(8) Simultaneously start the timer and place the superabsorbent particles and cylinder assembly onto the screen in the fluid bath for an hour. The level in the bath should be at a height to provide at least a 1 cm positive head above the base of the cylinder;
(9) Gently swirl the sample to release any trapped air and ensure the superabsorbent particles are in contact with the fluid.
(10) Remove the cylinder from the fluid bath at a designated time interval and immediately place the cylinder on the vacuum apparatus (ported disk on the top of the AUL chamber) and remove excess interstitial fluid for 10 seconds;
(11) Wipe the exterior of the cylinder with paper toweling or tissue; and
(12) Weigh the AUL assembly (i.e., cylinder, piston and weight), with the superabsorbent particles and any absorbed test fluid immediately and record the weight as WET WEIGHT in grams to the nearest milligram and the time interval.

The “absorbent capacity” of the superabsorbent particles at a designated time interval is calculated in grams liquid by grams superabsorbent by the following formula:

(Wet Weight−Dry Weight)/(Dry Weight−Container Weight)

A minimum of three samples should be tested and the results averaged to determine the free swell gel bed permeability of the sample. The samples should be tested at 23° C. and 50% relative humidity.

### Centrifuge Retention Capacity (CRC) Test Method

The Centrifuge Retention Capacity (CRC) Test Method measures the ability of superabsorbent particles to retain liquid after being saturated and subjected to centrifugation under controlled conditions. The resultant retention capacity is stated as grams of liquid retained per gram weight of the sample (g/g) and is measured according to EDANA recommended test method WSP 241.3. The sample to be tested is prepared from particles that are prescreened through a U.S. standard 30-mesh screen and retained on a U.S. standard 50-mesh screen. The particles can be prescreened by hand or automatically and are stored in a sealed airtight container until testing. The retention capacity is measured by placing 0.2±0.005 grams of the prescreened sample into a water-permeable bag that will contain the sample while allowing a test solution (0.9 weight percent sodium chloride in distilled water) to be freely absorbed by the sample. A heat-sealable tea bag material, such as model designation 1234T heat sealable filter paper, can be suitable. The bag is formed by folding a 5-inch by 3-inch sample of the bag material in half and heat-sealing two of the open edges to form a 2.5-inch by 3-inch rectangular pouch. The heat seals can be about 0.25 inches inside the edge of the material. After the sample is placed in the pouch, the remaining open edge of the pouch can also be heat-sealed. Empty bags can be made to serve as controls. Three samples (e.g., filled and sealed bags) are prepared for the test. The filled bags are tested within three minutes of preparation unless immediately placed in a sealed container, in which case the filled bags must be tested within thirty minutes of preparation.

The bags are placed between two TEFLON® coated fiberglass screens having 3-inch openings (Taconic Plastics, Inc., Petersburg, N.Y.) and submerged in a pan of the test solution at 23° C., making sure that the screens are held down until the bags are completely wetted. After wetting, the samples remain in the solution for about 30±1 minutes, at which time they are removed from the solution and temporarily laid on a non-absorbent flat surface. For multiple tests, the pan should be emptied and refilled with fresh test solution after 24 bags have been saturated in the pan.

The wet bags are then placed into the basket of a suitable centrifuge capable of subjecting the samples to a g-force of about 350. One suitable centrifuge is a Heraeus LaboFuge 400 having a water collection basket, a digital rpm gauge, and a machined drainage basket adapted to hold and drain the bag samples. Where multiple samples are centrifuged, the samples can be placed in opposing positions within the centrifuge to balance the basket when spinning. The bags (including the wet, empty bags) are centrifuged at about 1,600 rpm (e.g., to achieve a target g-force of about 350), for 3 minutes. The bags are removed and weighed, with the empty bags (controls) being weighed first, followed by the bags containing the samples. The amount of solution retained by the sample, taking into account the solution retained by the bag itself, is the centrifuge retention capacity (CRC) of the sample, expressed as grams of fluid per gram of sample. More particularly, the centrifuge retention capacity is determined as:

CRC=[Sample Bag Weight After Centrifuge−Empty Bag Weight After Centrifuge−Dry Sample Weight]/Dry Sample Weight

Three samples should be tested, and the results averaged to determine the retention capacity (CRC) of the superabsorbent material. The samples should be tested at 23° C. and 50% relative humidity.

### Dry Void Volume Determination Test Method

Dry void volumes of absorbent articles, portions of absorbent articles, materials, and absorbent layers can be calculated from total volume information, material composition information, material weight information, and density information. To determine the dry void volume of a sample, the following steps are performed:
(1) A thickness of a material sample is determined. The sample is prepared to have an area of greater than 90 mm by 102 mm. For exceptionally thin materials, multiple samples of the material may be stacked to achieve at least 1 mm in thickness. Using any commercially available thickness tester (for example the C640 Thickness Tester available from Labthink, having offices in Medford, MA), the thickness of the sample should be recorded to the nearest 0.01 mm, the thickness being the distance between the anvil or base of the tester and a platen applied to the sample at a controlled loading pressure of approximately 0.345 kPa. An exemplary platen has a diameter of approximately 7.5 cm.
(2) A total volume per unit area of the sample is then determined by dividing the measured thickness by 1.0 mm². As one illustrative example, the sample material may have a thickness of 1.5 mm. Accordingly, for a given 1 mm² area, the sample would have a total volume of 1.5 mm³ which translates to 1.5 mm³/mm². This value can be converted to a cm³/cm² value for ease of future calculations. For example, 1.5 mm³/mm²=0.0015 cm³/0.01 cm²=0.15 cm³/cm².
(3) A volume of the solids per unit area of the sample material is then determined utilizing the basis weight of the sample material (in grams per square meter—gsm or g/m²), the composition of the sample material, and a standard density of the sample material in g/cm³. In the present illustrative example, the sample material may have a basis weight of 100 gsm and be made of 50% polyester (PET) fibers by weight and 50% bicomponent fibers by weight (such bicomponent fibers comprising 50% polypropylene and 50% polyethylene). It is known that PET has a density of 1.38 g/cm³, and such bicomponent fibers have a density of 0.92 g/cm³. With these values, the average density of the fibers of the sample material is (1.38*0.50)+(0.92*0.50)=1.15 g/cm³. Accordingly, the volume of the solids per unit area of the sample material is then the basis weight of the material (100 g/m²) multiplied by the average density of the solids of the material (1.15 g/cm³). With unit conversions, the equation of 100 g/m² divided by 1.15 g/cm³=0.0087 cm³/cm².
(4) Finally, a dry void volume of the material per unit area (simply called dry void volume) may be determined by subtracting the determined volume of the solids per unit area of the sample material from the total volume per unit area. In the present illustrative example, the dry void volume of the sample material would be 0.15 cm³/cm² —0.0087 cm³/cm²=0.141 cm³/cm².

To determine dry void volume of composite materials or components of composite materials—for example a composite absorbent structure comprising upper and lower web materials with absorbent material and adhesive disposed between upper and lower web materials—the Steps 1-4 can be performed with respect to the composite material. In these instances, the volumes of solids per unit area of each of the various known components may be subtracted from the determined total volume per unit area of the composite to arrive at the dry void volume of the composite. Further, subtractions of the known dry void volumes of the individual components may be performed to arrive at dry void volumes of sub-structures of the composite or even individual components of the composite—for example the absorbent layer.

As one illustrative example used herein for clarity of how to calculate a dry void volume of a composite structure, an illustrative a composite absorbent structure (for instance, structure 101) may include an upper web material, a lower web material, and a superabsorbent and adhesive mixture disposed between the upper web material and the lower web material. According to Step 1, the thickness of the composite structure may be determined, and the volume per unit area may be determined according to Step 2. In this illustrative example, the thickness of the composite structure is 1.96 mm, and the volume per unit area is 0.196 cm³/cm².

To determine the dry void volume of the composite structure, an average density of all of the components of the composite structure is required. In the present illustrative example, the average density of the fibers of the upper and lower web materials is 0.93 g/cm³—again, which may come from known information about the fiber composition and fiber types and amounts forming the upper and lower web materials. The density of the superabsorbent material is 1.56 g/cm³—which may come from known information about the superabsorbent material or may be readily determined, such as according to a simple displacement test to determine a volume and a weight measurement according to a precision balance. The density of the adhesive is 1.0 g/cm³—which may come from known information about the adhesive composition. The basis weights of each of the components must be known as well. In the present example, the basis weight of the superabsorbent material is 460 gsm, the basis weight of each of the upper and lower web material is 20 gsm, and the basis weight of the adhesive is 23 gsm, giving a total basis weight of the composite of 523 gsm.

With the composite basis weight and the composite average density, the volume of the solids per unit area of the composite structure may be determined. The average density of the composite is according to the calculation: (1.56 g/cm³*(460 gsm/523 gsm)) plus (1.0 g/cm³*(23 gsm/523 gsm)) plus (0.93 g/cm³*(20 gsm gsm/523 gsm)) plus (0.93 g/cm³*(20 gsm gsm/523 gsm))=1.49 g/cm³. This average density of the composite structure may then be multiplied by the basis weight of the composite structure to result in the volume of solids per unit area of the composite structure: (with unit conversions) 523 g/m² divided by 1.49 g/cm³=0.0352 cm³/cm².

Finally, as in Step 4 above, the dry void volume of the composite structure may be determined as the total volume per unit area (0.196 cm³/cm²) minus the volume of solids per unit area of the composite structure (0.0352 cm³/cm²). In this example, the dry void volume is 0.161 cm³/cm². Additionally, dry void volume of the individual components may be determined by subtracting the known dry void volumes of the components from the dry void volume of the absorbent composite. As one example, the dry void volume of just the superabsorbent and adhesive mixture (the absorbent layer) is 0.161 cm³/cm² minus the dry void volumes of the upper and lower web materials (which may be determined in separate measurements according to this test method). In the present illustrative example, the dry void volumes of the upper and lower web materials are each 0.024 cm³/cm². For example, each of the upper and lower web materials are 0.285 mm thick, giving a total volume per unit area of 0.0285 cm³/cm² for each web. The web materials have basis weights of 20 gsm and densities of 0.93 g/cm³ yielding a volume of solids per unit area of: 20 g/m² divided by 0.93 g/cm³ equals 0.00215 cm³/cm². Subtracting the volume of solids per unit area of 0.00215 cm³/cm² for each of the web materials from the total volume per unit area of 0.0285 cm³/cm² results in a void volume of each of the upper and lower web materials of 0.0242 cm³/cm². Then, the dry void volume of just the superabsorbent and adhesive mixture (e.g. the absorbent layer) would be the total composite dry void volume of 0.161 cm³/cm² minus the dry void volume of the upper web material of 0.0242 cm³/cm² minus the dry void volume of the upper web material of 0.0242 cm³/cm²=0.112 cm³/cm².

The measurements of this test method should be performed on samples conditioned at 23° C. and 50% relative humidity. Additionally, for each dry void volume measurement, at least three (3) samples should be tested and the results averaged to obtain the dry void volume for a particular material or composite.

### Cradle Intake Test Method

The Cradle Intake Test Method is performed on whole products. The Cradle Intake Test Method utilizes a test cradle apparatus simulating the body curvature of a child. The apparatus consists of a plexiglass box having a width of 33 cm, height of 19 cm, and a length of 30.5 cm. The box has a first internal plexiglass wall extending from a top front edge of the box downward toward the bottom of the box and toward a transverse centerline of the box. The first internal wall extends at an angle of approximately 60 degrees from horizontal. The apparatus consists of a second internal plexiglass wall extending from a top back edge of the box downward toward the bottom of the box and toward the transverse centerline of the box. The second internal wall also extends at an angle of approximately 60 degrees from horizontal. The first internal wall and the second internal wall meet at the bottom of the box proximate the transverse centerline of the box and form a rounded connection having a radius of curvature of approximately 3.8 cm—thereby simulating the body curvature of a child. A slot having a length of 6.5 mm is positioned at the bottom of the box and forms a separation between the first internal wall and the second internal wall. A tray may be placed under the plexiglass box to capture run-off fluid that enters the slot. The first and second internal walls extend substantially from a first side edge of the box to a second side edge of the box.

For each single sample, the sample is opened to a flat (side seams are severed if relevant) and stretched-out configuration and a center of the product is marked—utilizing a ruler or other measuring device to find the center. Next, an insult point is marked. For a size 4 diaper or diaper pant, the insult point is marked as 8.5 cm forward (toward a front of the article) from the marked center.

Prior to being placed within the cradle, the sample product is weighed to the nearest 0.1 g using a suitable measurement device—for example an electro-balance readable to 0.01 g. If the product includes containment flaps, the flaps should be slightly lifted from the liner by running a finger underneath the containment flaps to ensure that no portion of the flaps are adhered to the liner.

The product is placed in the cradle positioned such that the longitudinal center of the product is located at the transverse centerline of the box with the liner facing up. The product is adhered to the first and second internal walls with tape (including double sided tape) to ensure the sample lays flat against eh internal walls. An end of a clear tubing—such as Masterflex clear tubing L/S 16, having an exit diameter of approximately 3 mm—is positioned approximately 1 cm away from the marked target insult location, with the tubing end pointing directly at the target insult location.

The clear tubing is connected to a suitable pump apparatus that is capable of controlling a flow rate (for example, a Cole-Parmer peristaltic pump P/N 07551-20, with pump head P/N 77201-60). The pump is connected to a reservoir containing a saline solution (0.9±0.0005% (w/w) aqueous isotonic saline) heated to 37±1 degree C.

For a size 4 product, the total volume per insult is 85 ml, and the fluid is delivered at a rate of 15 ml/s. Accordingly, the pump is set to appropriately control to these parameters. Utilizing a stopwatch, an insult is timed, beginning when fluid contacts the sample. The timer is stopped once all of the insulted liquid has penetrated below the surface of the sample, and the time is recorded to the nearest 0.01 seconds. Failures can occur whereby more than a few drops of liquid migrate to non-absorptive portions of the sample, where the liquid runs into the cradle (for example, by leaking from the sample), and whereby fluid pools for longer than 5 minutes. These failures should be noted and the data not included in the reported results. The recorded time is the First Cradle Intake Time.

The measurements of this test method should be performed on samples conditioned at 23° C. and 50% relative humidity. Additionally, for first cradle intake time measurement, at least four (4) samples should be tested, and the results averaged to obtain the first cradle intake time measurement.

### Percent Void Volume Increase Test Method

Initially, an absorbent body or structure is obtained. Where the absorbent body/structure is part of a larger structure—such as an absorbent article—the absorbent body/structure is removed from the absorbent article. Other components of the absorbent article may be carefully peeled away from the absorbent body/structure—such as the backsheet/outer cover, the liner, and any surge or acquisition/distribution layer. If required, commercially available freeze-spray type adhesive remover/deactivator may be utilized to help separate the absorbent body/structure from the other materials of the article.

With the absorbent body/structure separate, a single 76 mm (3) circle of the absorbent body/structure is cut with its center proximate a typical insult location for the absorbent body/structure or absorbent article type and size. For example, for size 4 open diaper and diaper pant absorbent articles, a typical insult location is 8.5 cm closer to the front waist edge of the article than a center of the absorbent article. This location may be readily translated to locations on the absorbent body/structure. This method may utilize a sample size of at least three (3), for which six (6) times the sample size separate circular samples should be cut from separate sample absorbent bodies/structures. For example, in where the sample size is three (3), eighteen (18) separate circular samples should be cut from separate sample absorbent bodies/structures. Additionally, the measurements of this test method should be performed on samples conditioned at 23° C. and 50% relative humidity.

Each of the dry circular absorbent body/structure samples are weighed and the weights recorded. Single dry circular absorbent body/structure samples are placed within individual waterproof containers having flat bottom of known thickness. The containers should be wide enough to allow the sample to lay flat and tall enough to contain a maximum quantity of fluid used for this test. The thickness of each of the samples is measured while in the waterproof containers, as according to Step 1 of the Dry Void Volume Determination Test Method. The thickness of the dry sample is then calculated by subtracting the known thickness of the waterproof containers.

Once the dry circular absorbent body/structure samples have been weighed and thicknesses determined, a number of the dry circular absorbent body/structure samples equal to the desired sample size (for example, three) are chosen to determine how much 0.9% saline needs to be added to each to obtain a gram per gram loading of 30 g/g for each of the chosen samples. For instance, if a first of the three samples was weighed to be 3.5 g, the sample would need to be loaded with 105 g of 0.9% saline to achieve a 30 g/g loading. Similarly, if a second of the three samples was weighed to be 3.3 g, the sample would need to be loaded with 99 g of 0.9% saline to achieve a 30 g/g loading. If the third of the three samples was weighed to be 3.6 g, the third sample would need to be loaded with 108 g of 0.9% saline to achieve a 30 g/g loading. Corresponding sets equal to the sample size of the dry circular absorbent body/structure samples are then chosen for loadings of 0 g/g, 5 g/g, 10 g/g, and 20 g/g. The amount of 0.9% saline needed to be added to each of the chosen samples for their chosen loading is then determined. It should be noted that a dry void volume at the 0 g/g loading of a dry circular sample is obtained according to this Percent Void Volume Increase Test Method.

With a circular sample in an individual waterproof container, the sample is then loaded with the determined amount of 0.9% saline needed to achieve the specific loading for a given sample. The fluid should be gently poured onto the circular sample in an even manner. The sample should sit for 20 min. once all of the fluid has been applied to the sample.

After the 20 minutes has elapsed, the thickness of the circular sample and the waterproof container, now loaded with an amount of 0.9% saline, is measured—as according to Step 1 of the Dry Void Volume Determination Test Method. The thickness of the circular sample is then determined by subtracting the known thickness of the waterproof containers. This fluid loading and thickness measuring process is repeated for each of the circular samples, being careful to utilize the amount of fluid determined to reach the determined g/g loading for the given sample—as the g/g loading will vary based on the exact weight of the individual sample.

With the thickness measurement for each of the circular samples, the partially saturated void volume may be determined for each circular sample. First, the volume per unit area of the sample is determined utilizing the measured thickness. As one example, where the thickness of the sample is measured to be 2.3 mm, the total volume per unit area is determined to be 2.3 mm³, which translates to 2.3 mm³/mm², which in turn can be converted to 0.23 cm³/cm². Next, the volume of solids per unit area of the circular sample is determined according to Step 2 of the Dry Void Volume Determination Test Method as described for the absorbent composite example of the Dry Void Volume Determination Test Method. For instance, an average density of the solids of a circular absorbent body/structure sample is determined according to the known densities of each of the materials and the basis weights of the materials (e.g. by summing up the densities of the individual components multiplied by their weight proportion in the total sample). Diving the total basis weight of the circular absorbent body/structure sample by the average density results in the volume of solids per unit area of the sample. Finally, the partially saturated void volume of the partially saturated circular sample is determined by subtracting out the determined volume of solids per unit area of the partially saturated circular sample from the total volume per unit area measurement. In this manner, the partially saturated void volume for each circular sample is determined.

In order to determine a percent partial saturation, a total capacity for the samples needs to be determined. To determine the total capacity for the absorbent body/structure, a complete absorbent body/structure sample (e.g. an absorbent body/structure without any circular samples cut-out) is obtained. The dry, complete absorbent body/structure sample is weighed on a precision balance to the nearest 0.01 g. Next, the complete absorbent body/structure sample is placed flat onto a polyethylene mesh screen and lowered into a bath of testing fluid of 0.9% saline with sufficient excess fluid to fully saturate the absorbent body/structure sample (for example, such that the tub has a depth of at least 50 mm of fluid). If the sample floats, push the sample into the testing fluid until completely submerged.

The absorbent body/structure sample is left for 20 min. Once 20 min. has elapsed, the saturated absorbent body/structure is removed from the testing fluid bath and placed on a Teflon™ coated fiberglass screen having 6.4 mm openings (commercially available from Taconic Plastics Inc., Petersburg, N.Y.) which, in turn, is placed on a vacuum box and covered with a flexible rubber dam material—for example, a latex sheet. The latex sheet should be slightly larger than the vacuum box in size and should seal to the box surrounding the sample during the test. The absorbent body/structure sample should be lifted by the front and rear product edges and kept as flat as possible while transferring to the fiberglass screen.

Once the sample is positioned on the vacuum box, a pump connected to the box is turned on and controlled to reach a vacuum of 3.45 kPa within the box. Once the vacuum of 3.45 kPa is reached, the absorbent body/structure sample is left for 5 minutes. After the vacuum of 3.45 kPa has been maintained for 5 minutes, the pump should be shut off. Then, the latex sheet is removed, and the absorbent body/structure sample is carefully removed from the vacuum table and placed onto the precision balance to obtain a fully saturated weight—recorded to the nearest 0.01 g.

This total capacity test is repeated for the desired sample size of the Percent Void Volume Increase Test Method (again, a minimum of three samples should be utilized). Once all of the samples have been measured, a total fluid retention weight is determined by subtracting the measured fully saturated absorbent body/structure sample weight from the measured dry absorbent body/structure sample weight with the results of the samples averaged. The maximum fluid retention weight for each circular sample can then be determined by diving the area of the circular samples (e.g. 76 mm diameter divided by 2 equals 38 mm radius, giving an area of 45.4 cm²) by the area of the absorbent body/structure sample and multiplying the result by the total fluid retention weight. As one illustrative example, where the absorbent body/structure sample has a length of 35.3 cm and a width of 8 cm (equaling an area of 282.4 cm²) and a total fluid retention weight of 470 g, the maximum fluid retention weight for each of the circular samples is 45.4 cm² divided by 282.4 cm² and multiplying the result of 0.161 by the total fluid retention weight of 470 g—which equals a maximum fluid retention weight for the circular samples of 75.6 g.

Finally, the percent saturation for each of the circular samples is determined by dividing the loaded weight of the 0.9% saline fluid by the determined maximum fluid retention weight. For instance, in the above example where the maximum fluid retention weight is 75.6 g, the percent saturation of the 5 g/g loading circular samples is the weight of the loaded fluid divided by 75.6 g. As on illustrative example, where a dry weight of a circular sample used for a 5 g/g loading is 3.4 g, the loading fluid weight is 3.4 multiplied by 5, equaling 17 g of loading fluid. This 17 g of loading fluid is 22.5% of the maximum fluid retention weight of the circular sample (17 g divided by 75.6 g, expressed as a percent). It should be noted that since the amount of 0.9% saline required to reach one or more of the loading levels (e.g. the 20 g/g or 30 g/g loading levels) of the circular samples may exceed the maximum weight of fluid retention of the circular samples, this percent saturation value may be greater than 100% in some instances.

All documents cited in the Detailed Description are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by references, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### EMBODIMENTS

Embodiment 1: An absorbent article may comprise a bodyside liner, a backsheet, and an absorbent structure disposed between the bodyside liner and the backsheet and comprising a top corewrap material and a bottom corewrap material with superabsorbent particles disposed between the top corewrap material and the bottom corewrap material, the superabsorbent particles disposed at a basis weight of greater than or equal to 250 gsm and present in an amount greater than or equal to 90% by weight of absorbent material within the absorbent article, wherein the superabsorbent particles have a vortex time of less than or equal to 41 s, according to the Vortex Time Test Method, the absorbent article has a dry void volume of less than 0.45 cm³/cm², as determined by the Dry Void Volume Determination Test Method, and the absorbent article has a first cradle intake time of less than or equal to 20 s, according to the Cradle Intake Test Method.
Embodiment 2: The absorbent article of embodiment 1, wherein the absorbent article may be cellulose-free.
Embodiment 3: The absorbent article of any one of embodiments 1 or 2, wherein the absorbent article may have a dry void volume of less than 0.40 cm³/cm².
Embodiment 4: The absorbent article of any one of embodiments 1-3, wherein the absorbent article may have a dry void volume of less than 0.35 cm³/cm².
Embodiment 5: The absorbent article of any one of embodiments 1-4, wherein the superabsorbent particles may have a vortex time of less than or equal to 29 s.
Embodiment 6: The absorbent article of embodiment 5, wherein the absorbent article may have a first cradle intake time of less than or equal to 18 s.
Embodiment 7: The absorbent article of any one of embodiments 1-6, wherein the superabsorbent particles may be intermixed with adhesive filaments prior to deposition onto the one of the top corewrap material and the bottom corewrap material, with the adhesive filaments forming a three-dimensional mesh network comprising network adhesive filaments, and wherein the superabsorbent particles are immobilized within the mesh network with the network adhesive filaments and superabsorbent particles extending throughout a three-dimensional space defined by the network adhesive filaments and the superabsorbent particles, and wherein the network adhesive filaments extend in random orientations throughout the three-dimensional space.
Embodiment 8: The absorbent article of any one of embodiments 1-7, wherein the adhesive may be disposed within the absorbent structure at an amount greater than 0% and less than or equal to 4%, by total weight of the superabsorbent particles.
Embodiment 9: The absorbent article of embodiment 8, wherein the absorbent structure may have a SAM Capture Value greater than or equal to 98, according to the SAM Capture Test Method.
Embodiment 10: An absorbent article may comprise a bodyside liner, a backsheet, and an absorbent structure disposed between the bodyside liner and the backsheet and comprising a top corewrap material and a bottom corewrap material with superabsorbent particles and adhesive disposed between the top corewrap material and the bottom corewrap material forming one or more absorbent layers, the superabsorbent particles disposed at a basis weight of greater than or equal to 250 gsm and present in an amount greater than or equal to 90% by weight of absorbent material of the absorbent article, wherein the superabsorbent particles have a vortex time of less than or equal to 41 s, according to the Vortex Time Test Method, the absorbent article has a dry void volume of less than 0.45 cm³/cm² and an absorbent structure, not including any corewrap materials, dry void volume of less than or equal to 0.19 cm³/cm², as determined by the Dry Void Volume Determination Test Method, and wherein the absorbent article has a first cradle intake time of less than or equal to 25 s, according to the Cradle Intake Test Method.
Embodiment 11: The absorbent article of embodiment 10, wherein the absorbent article may have a first cradle intake time of less than or equal to 20 s, according to the Cradle Intake Test Method.
Embodiment 12: The absorbent article of any one of embodiments 10 or 11, wherein the absorbent article may have a first cradle intake time of less than or equal to 15 s, according to the Cradle Intake Test Method.
Embodiment 13: The absorbent article of any one of embodiments 10-12, wherein the absorbent article may have a dry void volume of less than 0.40 cm³/cm², as determined by the Dry Void Volume Determination Test Method
Embodiment 14: The absorbent article of any one of embodiments 10-13, wherein the absorbent article may have a dry void volume of less than 0.35 cm³/cm², as determined by the Dry Void Volume Determination Test Method
Embodiment 15: The absorbent article of any one of embodiments 10-14, wherein the superabsorbent particles may have a vortex time of less than or equal to 29 s, according to the Vortex Time Test Method.
Embodiment 16: The absorbent article of any one of embodiments 10-15, wherein a combined dry void volume value of the one or more absorbent layers may increase by at least 180% at 20% saturation, according to the Percent Void Volume Increase Test Method.
Embodiment 17: The absorbent article of any one of embodiments 10-16, wherein the adhesive may be disposed within the absorbent structure at an amount greater than 0% and less than or equal to 5%, by total weight of the superabsorbent particles, and wherein the absorbent structure has a SAM Capture Value greater than or equal to 98, according to the SAM Capture Test Method.
Embodiment 18: The absorbent article of embodiment 17, wherein the superabsorbent particles are disposed at a basis weight of between 400 gsm and 600 gsm.
Embodiment 19: An absorbent article may comprise a bodyside liner, a backsheet, and an absorbent structure disposed between the bodyside liner and the backsheet and comprising a top corewrap material and a bottom corewrap material with superabsorbent particles and adhesive disposed between the top corewrap material and the bottom corewrap material forming one or more absorbent layers, the superabsorbent particles disposed at a basis weight of greater than or equal to 250 gsm and present in an amount greater than or equal to 90% by weight of absorbent material of the absorbent article, wherein the superabsorbent particles have a vortex time of less than or equal to 41 s, according to the Vortex Time Test Method, the absorbent article has a dry void volume of less than 0.45 cm³/cm², as determined by the Dry Void Volume Determination Test Method, and wherein a combined dry void volume value of the one or more absorbent layers increases by at least 314% at 40% saturation, according to the Percent Void Volume Increase Test Method.
Embodiment 20: The absorbent article of embodiment 19, wherein a combined dry void volume value of the one or more absorbent layers may increase by at least 359% at 40% saturation, according to the Percent Void Volume Increase Test Method.
Embodiment 21: The absorbent article of any one of embodiments 19 or 20, wherein a combined dry void volume value of the one or more absorbent layers may increase by at least 385% at 40% saturation, according to the Percent Void Volume Increase Test Method.
Embodiment 22: The absorbent article of any one of embodiments 19-21, wherein a combined dry void volume of the one or more absorbent layers may increase by at least 180% at 20% saturation, according to the Percent Void Volume Increase Test Method.
Embodiment 23: The absorbent article of any one of embodiments 19-22, wherein a combined dry void volume of the one or more absorbent layers may increase by at least 196% at 20% saturation, according to the Percent Void Volume Increase Test Method.
Embodiment 24: The absorbent article of any one of embodiments 19-23, wherein the absorbent structure, not including any corewrap materials, may have a dry void volume of less than or equal to 0.19 cm³/cm², as determined by the Dry Void Volume Determination Test Method.
Embodiment 25: The absorbent article of any one of embodiments 19-24, wherein the absorbent article may have a first cradle intake time of less than or equal to 25 s, according to the Cradle Intake Test Method.
Embodiment 26: The absorbent article of any one of embodiments 19-25, wherein the absorbent article may have a first cradle intake time of less than or equal to 20 s, according to the Cradle Intake Test Method.
Embodiment 27: The absorbent article of any one of embodiments 19-26, wherein the absorbent article may have a dry void volume of less than 0.40 cm³/cm², as determined by the Dry Void Volume Determination Test Method.
Embodiment 28: The absorbent article of any one of embodiments 19-27, wherein the absorbent article may have a dry void volume of less than 0.35 cm³/cm², as determined by the Dry Void Volume Determination Test Method.
Embodiment 29: An absorbent article may comprise a bodyside liner, a backsheet, and an absorbent structure disposed between the bodyside liner and the backsheet and comprising a top corewrap material and a bottom corewrap material with superabsorbent particles and adhesive disposed between the top corewrap material and the bottom corewrap material, the superabsorbent particles disposed at a basis weight of greater than or equal to 250 gsm and present in an amount greater than or equal to 90% by weight of absorbent material of the article, wherein the superabsorbent particles are intermixed with adhesive filaments prior to deposition onto the one of the top corewrap and bottom corewrap materials with the adhesive filaments forming a three-dimensional mesh network comprising network adhesive filaments, and wherein the superabsorbent particles are immobilized within the mesh network with the network adhesive filaments and superabsorbent particles extending throughout a three-dimensional space defined by the network adhesive filaments and the superabsorbent particles with the network adhesive filaments extending in random orientations throughout the three-dimensional space, wherein the superabsorbent particles have a vortex time of less than 41 s, according to the Vortex Time Test Method, the absorbent article has a dry void volume of less than 0.45 cm³/cm², as determined by the Dry Void Volume Determination Test Method, and wherein the absorbent article is configured to increase in total void volume and reach a total void volume of at least 0.841 cm³/cm² at 40%, according to the Percent Void Volume Increase Test Method.
Embodiment 30: The absorbent article of embodiment 29, wherein the absorbent article may be configured to increase in total void volume and reach a total void volume of greater than 0.620 cm³/cm² at 20%, according to the Percent Void Volume Increase Test Method.
Embodiment 31: The absorbent article of any one of embodiment 29 or 30, wherein the absorbent article may be configured to increase in total void volume and reach a total void volume of greater than 0.715 cm³/cm² at 20%, according to the Percent Void Volume Increase Test Method.
Embodiment 32: The absorbent article of embodiment 31, wherein the absorbent article may be configured to increase in total void volume and reach a total void volume of greater than or equal to 0.907 cm³/cm² at 40%, according to the Percent Void Volume Increase Test Method.
Embodiment 33: The absorbent article of any one of embodiments 31 or 32, wherein the absorbent article may have a dry void volume of less than 0.270 cm³/cm².
Embodiment 34: The absorbent article of embodiment 33, wherein the absorbent article may have a first cradle intake time of less than or equal to 20 s, according to the Cradle Intake Test Method.
Embodiment 35: The absorbent article of any one of embodiments 29-34, wherein the adhesive may be disposed within the absorbent structure at an amount greater than 0% and less than or equal to 5%, by total weight of the superabsorbent particles, and wherein the absorbent structure has a SAM Capture Value greater than or equal to 98, according to the SAM Capture Test Method.
Embodiment 36: The absorbent article of embodiment 35, wherein the superabsorbent particles may be disposed at a basis weight of between 400 gsm and 600 gsm.
Embodiment 37: An absorbent article may comprise a bodyside liner, a backsheet, and an absorbent structure disposed between the bodyside liner and the backsheet and comprising a top corewrap material and a bottom corewrap material with superabsorbent particles and adhesive disposed between the top corewrap material and the bottom corewrap material, the superabsorbent particles disposed at a basis weight of greater than or equal to 250 gsm and present in an amount greater than or equal to 90% by weight of absorbent material of the article, wherein the superabsorbent particles are intermixed with adhesive filaments prior to deposition onto the one of the top corewrap and bottom corewrap materials with the adhesive filaments forming a three-dimensional mesh network comprising network adhesive filaments, and wherein the superabsorbent particles are immobilized within the mesh network with the network adhesive filaments and superabsorbent particles extending throughout a three-dimensional space defined by the network adhesive filaments and the superabsorbent particles with the network adhesive filaments extending in random orientations throughout the three-dimensional space, wherein the superabsorbent particles have a vortex time of less than or equal to 41 s, according to the Vortex Time Test Method, the absorbent article has a dry void volume of less than or equal to 0.40 cm³/cm² and the absorbent structure, not including any corewrap materials, has a dry void volume of less than 0.19 cm³/cm², as determined by the Dry Void Volume Determination Test Method, and wherein the absorbent structure is configured to increase in void volume and reach a total void volume of at least 0.46 cm³/cm² at 40%, according to the Percent Void Volume Increase Test Method.
Embodiment 37: The absorbent article of Embodiment 37, wherein the absorbent article may be configured to increase in total void volume and reach a total void volume of at least 0.51 cm³/cm² at 40%, according to the Percent Void Volume Increase Test Method.
Embodiment 38: The absorbent article of any one of embodiments 36 or 37, wherein the absorbent article may be configured to increase in total void volume and reach a total void volume of at least 0.33 cm³/cm² at 20%, according to the Percent Void Volume Increase Test Method.
Embodiment 39: The absorbent article of embodiment 38, wherein the absorbent article may be configured to increase in total void volume and reach a total void volume of at least 0.37 cm³/cm² at 20%, according to the Percent Void Volume Increase Test Method.
Embodiment 40: The absorbent article of any one of embodiments 38 or 40, wherein the absorbent article may have a dry void volume of less than 0.35 cm³/cm².
Embodiment 41: The absorbent article of embodiment 40, wherein the absorbent article may have a first cradle intake time of less than or equal to 20 s, according to the Cradle Intake Test Method.
Embodiment 42: The absorbent article of any one of embodiments 37-41, wherein the adhesive may be disposed within the absorbent structure at an amount greater than 0% and less than or equal to 5%, by total weight of the superabsorbent particles, and wherein the absorbent structure has a SAM Capture Value greater than or equal to 98, according to the SAM Capture Test Method.
Embodiment 43: The absorbent article of embodiment 42, wherein the superabsorbent particles may be disposed at a basis weight of between 400 gsm and 600 gsm.

What is claimed is:

1. An absorbent article comprising:
a bodyside liner;
a backsheet; and
an absorbent structure disposed between the bodyside liner and the backsheet and comprising a top corewrap material and a bottom corewrap material with superabsorbent particles disposed between the top corewrap material and the bottom corewrap material, the superabsorbent particles disposed at a basis weight of greater than or equal to 250 gsm and present in an amount greater than or equal to 90% by weight of absorbent material within the absorbent article;
wherein:
the superabsorbent particles have a vortex time of less than or equal to 41 s, according to the Vortex Time Test Method,
the absorbent article has a dry void volume of less than 0.45 cm³/cm², as determined by the Dry Void Volume Determination Test Method, and
the absorbent article has a first cradle intake time of less than or equal to 20 s, according to the Cradle Intake Test Method.

2. The absorbent article of claim 1, wherein the absorbent article is cellulose-free.

3. The absorbent article of claim 1, wherein the absorbent article has a dry void volume of less than 0.40 cm³/cm².

4. The absorbent article of claim 1, wherein the absorbent article has a dry void volume of less than 0.35 cm³/cm².

5. The absorbent article of claim 1, wherein the superabsorbent particles have a vortex time of less than or equal to 29 s.

6. The absorbent article of claim 5, wherein the absorbent article has a first cradle intake time of less than or equal to 18 s.

7. The absorbent article of claim 1, wherein the superabsorbent particles are intermixed with adhesive filaments prior to deposition onto the one of the top corewrap material and the bottom corewrap material, with the adhesive filaments forming a three-dimensional mesh network comprising network adhesive filaments, and wherein the superabsorbent particles are immobilized within the mesh network with the network adhesive filaments and superabsorbent particles extending throughout a three-dimensional space defined by the network adhesive filaments and the superabsorbent particles, and wherein the network adhesive filaments extend in random orientations throughout the three-dimensional space.

8. The absorbent article of claim 1, wherein the adhesive is disposed within the absorbent structure at an amount greater than 0% and less than or equal to 4%, by total weight of the superabsorbent particles.

9. The absorbent article of claim 8, wherein the absorbent structure has a SAM Capture Value greater than or equal to 98, according to the SAM Capture Test Method.

10. An absorbent article comprising:
a bodyside liner;
a backsheet; and
an absorbent structure disposed between the bodyside liner and the backsheet and comprising a top corewrap material and a bottom corewrap material with superabsorbent particles and adhesive disposed between the top corewrap material and the bottom corewrap material forming one or more absorbent layers, the superabsorbent particles disposed at a basis weight of greater than or equal to 250 gsm and present in an amount greater than or equal to 90% by weight of absorbent material of the absorbent article,
wherein:
the superabsorbent particles have a vortex time of less than or equal to 41 s, according to the Vortex Time Test Method,
the absorbent article has a dry void volume of less than 0.45 cm³/cm² and an absorbent structure dry void volume, not including any corewrap materials, of less than or equal to 0.19 cm³/cm², as determined by the Dry Void Volume Determination Test Method, and
wherein the absorbent article has a first cradle intake time of less than or equal to 25 s, according to the Cradle Intake Test Method.

11. The absorbent article of claim 10, wherein the absorbent article has a first cradle intake time of less than or equal to 20 s, according to the Cradle Intake Test Method.

12. The absorbent article of claim 10, wherein the absorbent article has a first cradle intake time of less than or equal to 15 s, according to the Cradle Intake Test Method.

13. The absorbent article of claim 10, wherein the absorbent article has a dry void volume of less than 0.40 cm³/cm², as determined by the Dry Void Volume Determination Test Method

14. The absorbent article of claim 10, wherein the absorbent article has a dry void volume of less than 0.35 cm³/cm², as determined by the Dry Void Volume Determination Test Method

15. The absorbent article of claim 10, wherein the superabsorbent particles have a vortex time of less than or equal to 29 s, according to the Vortex Time Test Method.

16. The absorbent article of claim 10, wherein a combined dry void volume value of the one or more absorbent layers increases by at least 180% at 20% saturation, according to the Percent Void Volume Increase Test Method.

17. The absorbent article of claim 10, wherein the adhesive is disposed within the absorbent structure at an amount greater than 0% and less than or equal to 5%, by total weight of the superabsorbent particles, and wherein the absorbent structure has a SAM Capture Value greater than or equal to 98, according to the SAM Capture Test Method.

18. The absorbent article of claim 17, wherein the superabsorbent particles are disposed at a basis weight of between 400 gsm and 600 gsm.

19. An absorbent article comprising:
a bodyside liner;
a backsheet; and
an absorbent structure disposed between the bodyside liner and the backsheet and comprising a top corewrap material and a bottom corewrap material with superabsorbent particles and adhesive disposed between the top corewrap material and the bottom corewrap material forming one or more absorbent layers, the superabsorbent particles disposed at a basis weight of greater than or equal to 250 gsm and present in an amount greater than or equal to 90% by weight of absorbent material of the absorbent article,
wherein:
the superabsorbent particles have a vortex time of less than or equal to 41 s, according to the Vortex Time Test Method,
the absorbent article has a dry void volume of less than 0.45 cm³/cm², as determined by the Dry Void Volume Determination Test Method, and
wherein a combined dry void volume value of the one or more absorbent layers increases by at least 314% at 40% saturation, according to the Percent Void Volume Increase Test Method.

20. The absorbent article of claim 19, wherein a combined dry void volume value of the one or more absorbent layers increases by at least 359% at 40% saturation, according to the Percent Void Volume Increase Test Method.

21. The absorbent article of claim 19, wherein a combined dry void volume value of the one or more absorbent layers increases by at least 385% at 40% saturation, according to the Percent Void Volume Increase Test Method.

22. The absorbent article of claim 19, wherein a combined dry void volume of the one or more absorbent layers increases by at least 180% at 20% saturation, according to the Percent Void Volume Increase Test Method.

23. The absorbent article of claim 19, wherein a combined dry void volume of the one or more absorbent layers increases by at least 196% at 20% saturation, according to the Percent Void Volume Increase Test Method.

24. The absorbent article of claim 19, wherein the absorbent structure, not including any corewrap materials, has a dry void volume of less than or equal to 0.19 cm³/cm², as determined by the Dry Void Volume Determination Test Method.

25. The absorbent article of claim 19, wherein the absorbent article has a first cradle intake time of less than or equal to 25 s, according to the Cradle Intake Test Method.

26. The absorbent article of claim 19, wherein the absorbent article has a first cradle intake time of less than or equal to 20 s, according to the Cradle Intake Test Method.

27. The absorbent article of claim 19, wherein the absorbent article has a dry void volume of less than 0.40 cm³/cm², as determined by the Dry Void Volume Determination Test Method.

28. The absorbent article of claim 19, wherein the absorbent article has a dry void volume of less than 0.35 cm³/cm², as determined by the Dry Void Volume Determination Test Method.
